SEQUENCE LISTING (30 sequences)

SEQ ID NO: 1 - DNA, 29 bp, synthetic construct
  source 1..29: mol_type = other DNA
tcattatttttattataaggcctgctgaa

SEQ ID NO: 2 - DNA, 23 bp, synthetic construct
  source 1..23: mol_type = other DNA
caaagaatggtcctgcaccagta

SEQ ID NO: 3 - DNA, 26 bp, synthetic construct
  source 1..26: mol_type = other DNA
tcttgcctacgccaccagctccaact

SEQ ID NO: 4 - DNA, 23 bp, synthetic construct
  source 1..23: mol_type = other DNA
tgcttgctctgataggaaaatga

SEQ ID NO: 5 - DNA, 22 bp, synthetic construct
  source 1..22: mol_type = other DNA
ccacaaaatggatccagacaac

SEQ ID NO: 6 - DNA, 23 bp, synthetic construct
  source 1..23: mol_type = other DNA
tggtctagctacagtgaaatctc

SEQ ID NO: 7 - DNA, 17 bp, synthetic construct
  source 1..17: mol_type = other DNA
gtggcaagtggctcctg

SEQ ID NO: 8 - DNA, 22 bp, synthetic construct
  source 1..22: mol_type = other DNA
ctcatcttgggcctgtgttatc

SEQ ID NO: 9 - DNA, 26 bp, synthetic construct
  source 1..26: mol_type = other DNA
atgggcctccggttcatgtcgcctat

SEQ ID NO: 10 - DNA, 26 bp, synthetic construct
  source 1..26: mol_type = other DNA
atgggcctccggttcatgtcgcctat

SEQ ID NO: 11 - DNA, 24 bp, synthetic construct
  source 1..24: mol_type = other DNA
agttttttaatacggatttcgtgc

SEQ ID NO: 12 - DNA, 21 bp, synthetic construct
  source 1..21: mol_type = other DNA
cctcctaacaaaaccaacgaa

SEQ ID NO: 13 - DNA, 38 bp, synthetic construct
  source 1..38: mol_type = other DNA
  modified_base 19: mod_base = OTHER; note = Locked nucleic acid
  modified_base 20: mod_base = OTHER; note = Locked nucleic acid
cctctcacacttgaggtgttacgggggcgttggagtat

SEQ ID NO: 14 - DNA, 23 bp, synthetic construct
  source 1..23: mol_type = other DNA
ggtttttacgtttttcgtagttc

SEQ ID NO: 15 - DNA, 21 bp, synthetic construct
  source 1..21: mol_type = other DNA
cgctcatctcctaaactcgaa

SEQ ID NO: 16 - DNA, 38 bp, synthetic construct
  source 1..38: mol_type = other DNA
ctctctctggtcgggatttggggttgagttttcgtcgc

SEQ ID NO: 17 - DNA, 39 bp, synthetic construct
  source 1..39: mol_type = other DNA
cctctcacacttcgggatttggggttgagttttcgtcgc

SEQ ID NO: 18 - DNA, 21 bp, synthetic construct
  source 1..21: mol_type = other DNA
ggttttgtgtcgttcgttttc

SEQ ID NO: 19 - DNA, 25 bp, synthetic construct
  source 1..25: mol_type = other DNA
acgataattatacgcctaaaactcg

SEQ ID NO: 20 - DNA, 33 bp, synthetic construct
  source 1..33: mol_type = other DNA
ctctctctggtgtgcgcgaggcgcggagtattt

SEQ ID NO: 21 - DNA, 25 bp, synthetic construct
  source 1..25: mol_type = other DNA
ttggttatagtcgttgttgtttttc

SEQ ID NO: 22 - DNA, 20 bp, synthetic construct
  source 1..20: mol_type = other DNA
cccgaatttcgatcccttcg

SEQ ID NO: 23 - DNA, 37 bp, synthetic construct
  source 1..37: mol_type = other DNA
  modified_base 14: mod_base = OTHER; note = Locked nucleic acid
  modified_base 15: mod_base = OTHER; note = Locked nucleic acid
tctggtctatcttttcggggttttcggttttcgagaa

SEQ ID NO: 24 - DNA, 22 bp, synthetic construct
  source 1..22: mol_type = other DNA
cgaatttttattcgggagatcg

SEQ ID NO: 25 - DNA, 22 bp, synthetic construct
  source 1..22: mol_type = other DNA
ccaaccctcctttcaaatatcg

SEQ ID NO: 26 - DNA, 34 bp, synthetic construct
  source 1..34: mol_type = other DNA
cacctctcacaaggcgggttgtttggttcgcgac

SEQ ID NO: 27 - DNA, 20 bp, synthetic construct
  source 1..20: mol_type = other DNA
gagtttagaagttcggttgc

SEQ ID NO: 28 - DNA, 18 bp, synthetic construct
  source 1..18: mol_type = other DNA
cccgactaaaaaacgccg

SEQ ID NO: 29 - DNA, 40 bp, synthetic construct
  source 1..40: mol_type = other DNA
cacctctcacagattcgggatttcggttttcgtttttcgt

SEQ ID NO: 30 - DNA, 43 bp, synthetic construct
  source 1..43: mol_type = other DNA
tctggtctatctttcgggatttcggttttcgtttttcgttagt

(19) United States
(12) Patent Application Publication
(10) Pub. No.: US 2025/0320556 A1
(43) Pub. Date: Oct. 16, 2025

(54) METHODS AND RELATED ASPECTS FOR DIGITAL MULTI-TEMPERATURE FLUOROMETRIC DETECTION

(71) Applicant: THE JOHNS HOPKINS UNIVERSITY, Baltimore, MD (US)

(72) Inventors: Tza-Huei Jeff WANG, Timonium, MD (US); Fan-En CHEN, Baltimore, MD (US); Yang ZHAO, Baltimore, MD (US); Weiwen CUI, Baltimore, MD (US); Jiumei HU, Baltimore, MD (US)

(21) Appl. No.: 19/174,275

(22) Filed: Apr. 9, 2025

Related U.S. Application Data
(60) Provisional application No. 63/633,112, filed on Apr. 12, 2024.

(51) Int. Cl. C12Q 1/6881; C12Q 1/686

(52) U.S. Cl. CPC C12Q 1/6881; C12Q 1/686; C12Q 2600/154; C12Q 2600/16

(57) ABSTRACT

Provided herein are methods of performing a high-level of multiplexed analyte detection, including analytes such as nucleic acids and proteins. The methods include performing a melting curve analysis using multiple probe sets under conditions sufficient to generate a melting temperature (Tₘ) detection barcode data set from partitioned sample aliquots. Related systems and computer program products are also provided.

### CROSS-REFERENCE TO RELATED APPLICATONS

This application claims priority to U.S. Provisional Patent Application Ser. No. 63/633,112, filed Apr. 12, 2024, the disclosure of which is incorporated herein by reference.

### STATEMENT REGARDING FEDERALLY SPONSORED RESEARCH OR DEVELOPMENT

This invention was made with government support under grants Al137272 and CA272321, awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 8, 2025, is named 0184_0300_SL.xml and is 29,610 bytes in size.

### BACKGROUND

Multiplex PCR-based detection of analytes involves the simultaneous detection of multiple target molecules in a single sample. This method has diverse applications, such as identifying various infectious agents that manifest similar clinical symptoms in the host, verifying assay performance through internal controls, and evaluating the expression of multiple biomarkers for disease diagnosis and genotyping in plants or animals (FIG. 3A). However, the current techniques for multiplexed analyte detection face limitations due to the restricted number of available color channels in instruments or the use of large, complex, and costly fluidic handling devices, which hinders their widespread adoption.

To enable simultaneous measurement of multiple analytes, analytes can be detected by utilizing electrochemical, fluorescent, or colorimetric labels in separate biochemical reactions or within a single reaction. Traditional approaches involve incorporating additional fluorescence channels to detect more targets labeled with distinct fluorophores or using fluidic control to divide a sample into multiple reactions such that a single reaction contains a single analyte. However, the former method is ultimately constrained by the limited selection of commercially available fluorophores, while the latter method faces limitations due to the need for large, complex, or expensive instruments for widespread use.

In recent years, a novel approach has been proposed to achieve multiplexing in the temperature domain. This method entails detecting targeted analytes by measuring the melting temperature hybridization with a fluorophore-labeled molecule probe, such as TaqMan probe or molecular beacon (FIGS. 3B and 3C). In the detection process, as the temperature of the reagent rises, the transition from hybridization to dissociation of the analyte and molecule probe is identified by observing a decrease in the fluorescent signal. This decrease generates a distinct melt peak in a specific color channel. However, this technique is ultimately limited by the number of fluorophore channels equipped and instrument's ability to detect subtle changes in melting temperature. Taking 2 fluorescence channels and 3 melting temperatures for barcoding as an example, the traditional method using individual melting curve for each target can only distinguish up to 6 targets (FIG. 4A).

Accordingly, there is a need for additional methods, and related aspects, for performing a high-level of multiplexed analyte detection, including analytes such as nucleic acids and proteins.

### SUMMARY

The present disclosure relates, in certain aspects, to methods, systems, and computer readable media of use in performing digital multi-temperature fluorometric detection. In some embodiments, the methods of the present disclosure couples a 3-dimensional melting curve labeling scheme and digital microfluidics to achieve a high-level of multiplexed detection. The level of multiplexing exponentially increases with an increasing number of fluorophores and an increasing number of melting curves fit in each fluorescence channel. As illustrated herein, for example, in some embodiments, the methods of the present disclosure can perform a 10,000 or more plex analysis in a single reaction using only a three-color detection system. In some embodiments, at least about 140 unique color-temperature codes can be generated using only those three colors. These and other aspects will be apparent upon a complete review of the present disclosure, including the accompanying figures.

In one aspect, the present disclosure provides a method of detecting multiple target nucleic acids in a nucleic acid sample. The method comprises performing a melting curve analysis using multiple probe sets under conditions sufficient to generate a melting temperature (Tₘ) detection barcode data set from partitioned sample aliquots created from the nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise: at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or, at least two mediator probe nucleic acids (sometimes referred to as a “flap-labeled probe”) and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and, identifying at least two different Tₘ detection barcodes in the Tₘ detection barcode data set, thereby detecting the multiple target nucleic acids.

In some embodiments, the method comprises generating the nucleic acid sample from a protein sample by: contacting the protein sample with a proximal binding probe set under conditions sufficient to produce a set of pairwise probe bound target proteins, wherein the proximal binding probe set comprises a plurality of proximal binding probe pairs, wherein a given proximal binding probe pair comprises a first binding probe comprising a first target protein binding moiety coupled to a first oligonucleotide that comprises a first hybridization site and a second binding probe comprising a second target protein binding moiety coupled to a second oligonucleotide that comprises a second hybridization site, wherein the first and second target protein binding moieties bind to different epitopes on a given target protein and wherein the first and second hybridization sites hybridize with one another when the first and second target protein binding moieties bind to the different epitopes on the given target protein to produce a given pairwise probe bound target protein; extending the first and second oligonucleotides in the set of pairwise probe bound target proteins to produce extended oligonucleotides in the set of pairwise probe bound target proteins; and, separating the extended oligonucleotides from the first and second target protein binding moieties in the set of pairwise probe bound target proteins, thereby generating the nucleic acid sample from the protein sample.

In some embodiments, identifying the at least two different Tₘ detection barcodes in the Tₘ data set thereby further detects multiple target proteins in the protein sample. In some embodiments, the proximal binding probe set comprises an oligonucleotide-coupled antibody probe. In some embodiments, the method comprises extending the mediator probes prior to dissociation from the universal reporter probe. In some embodiments, the reporter probe nucleic acid comprises a universal reporter probe nucleic acid. In some embodiments, the probe nucleic acids comprise labeled probe nucleic acids. In some embodiments, a given second subsequence bound to the reporter probe nucleic acid is extended prior to being dissociated from the reporter probe nucleic acid to produce the given Tₘ detection barcode for the given target nucleic acid.

In some embodiments, the method comprises performing a bulk PCR technique using the nucleic acid sample. In some embodiments, the method comprises performing an asymmetric PCR technique using the nucleic acid sample. In some embodiments, an emulsion comprises the partitioned sample aliquots. In some embodiments, the method comprises generating the Tₘ detection barcode data set using a microfluidic device. In some embodiments, the method comprises performing a digital PCR technique using the nucleic acid sample prior to and/or when identifying the at least two different Tₘ detection barcodes.

In some embodiments, the multiple probe sets comprise at least one probe set having a single probe nucleic acid that binds to a particular target nucleic acid in the partitioned sample aliquots and that comprises a melting temperature when dissociated from the particular target nucleic acid to produce a particular Tₘ detection barcode for the particular target nucleic acid. In some embodiments, the multiple probe sets comprise at least one probe set having a single mediator probe nucleic acid and the reporter probe nucleic acid, wherein the single mediator probe nucleic acid comprises a first subsequence that binds to a particular target nucleic acid in the partitioned sample aliquots and a second subsequence that binds to the reporter probe nucleic acid when cleaved from the single mediator probe nucleic acid, and wherein a cleaved second subsequence from the single mediator probe nucleic acid comprises a melting temperature when dissociated from the reporter probe nucleic acid to produce a particular Tₘ detection barcode for the particular target nucleic acid. In some embodiments, the nucleic acid sample comprises one or more bisulfite converted target nucleic acids and wherein the method further comprises identifying one or more methylation patterns in the bisulfite converted target nucleic acids. In some embodiments, the method comprises using one or more nucleic acids comprising a nucleotide sequence selected from SEQ ID NOS: 1-30 to generate at least a portion of the Tₘ detection barcode data set.

In some embodiments, one or more of the multiple probe sets comprise exonuclease probes. In some embodiments, one or more of the multiple probe sets comprise hairpin probes. In some embodiments, one or more of the multiple probe sets comprise hybridization probes. In some embodiments, the multiple probe sets are configured to generate a Tₘ detection barcode data set that comprise at least 3, at least 4, at least 5, at least 6, at least 7, at least 8, at least 9, at least 10, at least 15, at least 25, at least 50, at least 75, at least 100, at least 200, at least 300, at least 400, at least 500, at least 600, at least 700, at least 800, at least 900, at least 1000, or more different Tₘ detection barcodes. In some embodiments, the probe nucleic acids each comprise at least one fluorescent labeling moiety. In some embodiments, multiple probe nucleic acids in a given probe set comprise an identical fluorescent labeling moiety. In some embodiments, multiple probe nucleic acids in a given probe set comprise a different fluorescent labeling moiety.

In some embodiments, the multiple target nucleic acids comprise between about 3 and about 1000 different target nucleic acids, between about 4 and about 500 different target nucleic acids, between about 5 and about 100 different target nucleic acids, between about 6 and about 90 different target nucleic acids, between about 7 and about 80 different target nucleic acids, between about 8 and about 70 different target nucleic acids, between about 9 and about 60 different target nucleic acids, between about 10 and about 50 different target nucleic acids, between about 15 and about 40 different target nucleic acids, or between about 20 and about 30 different target nucleic acids.

In some embodiments, the method comprises obtaining the nucleic acid sample from a subject. In some embodiments, one or more of the multiple target nucleic acids that were detected identify the subject. In some embodiments, one or more of the multiple target nucleic acids that were detected are associated with at least one disease state in the subject. In some embodiments, the method comprises administering at least one therapy to the subject to treat the disease state in the subject. In some embodiments, the multiple target nucleic acids that were detected are from an infectious organism in the subject. In some embodiments, the multiple target nucleic acids that were detected are from nucleic acid variants associated with the disease state in the subject. In some embodiments, the disease state comprises a cancer type.

In another aspect, the present disclosure provides a A method of detecting multiple target nucleic acids in a nucleic acid sample. The method includes performing a melting curve analysis using multiple probe sets under conditions sufficient to generate a melting temperature (Tₘ) detection barcode data set from partitioned sample aliquots created from the nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise: at least one probe nucleic acid that binds to a given target nucleic acid in the partitioned sample aliquots and that comprises a melting temperature when dissociated from the given target nucleic acid to produce a given Tₘ detection barcode for the given target nucleic acid, or, at least one mediator probe nucleic acid and at least one reporter probe nucleic acid, wherein the mediator probe nucleic acid comprises a first subsequence that binds to a given target nucleic acid in the partitioned sample aliquots and a second subsequence that binds to the reporter probe nucleic acid when cleaved from the mediator probe nucleic acids, and wherein a cleaved second subsequence from the at least one mediator probe nucleic acid comprises a melting temperature when dissociated from the reporter probe nucleic acid to produce a given Tₘ detection barcode for the given target nucleic acid; and, identifying at least two different Tₘ detection barcodes in the Tₘ detection barcode data set, thereby detecting the multiple target nucleic acids.

In another aspect, the present disclosure provides a system that comprises a chamber configured to contain partitioned sample aliquots created from a nucleic acid sample; a thermal modulator configured to modulate temperature in the chamber to perform a melting curve analysis using multiple probe sets under conditions sufficient to generate a melting temperature (Tₘ) detection barcode data set from the partitioned sample aliquots created from a nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise: at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or, at least two mediator probe nucleic acids and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and, a detector configured to detect the Tₘ detection barcode data set; and a controller operably connected to the temperature modulator and to the detector, which controller comprises, or is capable of accessing, computer readable media comprising non-transitory computer-executable instructions which, when executed by at least one electronic processor perform at least: identifying at least two different Tₘ detection barcodes in the Tₘ detection barcode data set.

In some embodiments, the system further comprises a fluid handler operably connected to the controller, wherein the fluid handler and thermal modulator are configured to generate the nucleic acid sample from a protein sample by: contacting the protein sample with a proximal binding probe set under conditions sufficient to produce a set of pairwise probe bound target proteins, wherein the proximal binding probe set comprises a plurality of proximal binding probe pairs, wherein a given proximal binding probe pair comprises a first binding probe comprising a first target protein binding moiety coupled to a first oligonucleotide that comprises a first hybridization site and a second binding probe comprising a second target protein binding moiety coupled to a second oligonucleotide that comprises a second hybridization site, wherein the first and second target protein binding moieties bind to different epitopes on a given target protein and wherein the first and second hybridization sites hybridize with one another when the first and second target protein binding moieties bind to the different epitopes on the given target protein to produce a given pairwise probe bound target protein; extending the first and second oligonucleotides in the set of pairwise probe bound target proteins to produce extended oligonucleotides in the set of pairwise probe bound target proteins; and, separating the extended oligonucleotides from the first and second target protein binding moieties in the set of pairwise probe bound target proteins to thereby generate the nucleic acid sample from the protein sample.

In some embodiments, the non-transitory computer-executable instructions which, when executed by the electronic processor, further perform at least: detecting multiple target proteins in the protein sample when identifying the at least two different Tₘ detection barcodes in the Tₘ detection barcode data set. In some embodiments, the non-transitory computer-executable instructions which, when executed by the electronic processor, further perform at least: extending the mediator probes prior to dissociation from the universal reporter probe using the thermal modulator. In some embodiments, the non-transitory computer-executable instructions which, when executed by the electronic processor, further perform at least: performing a bulk PCR technique using the nucleic acid sample and the thermal modulator. In some embodiments, the non-transitory computer-executable instructions which, when executed by the electronic processor, further perform at least: performing an asymmetric PCR technique using the nucleic acid sample and the thermal modulator. In some embodiments, the non-transitory computer-executable instructions which, when executed by the electronic processor, further perform at least: performing a digital PCR technique using the nucleic acid sample prior to and/or when identifying the at least two different Tₘ detection barcodes using the thermal modulator. In some embodiments, the non-transitory computer-executable instructions which, when executed by the electronic processor, further perform at least: identifying one or more methylation patterns in the bisulfite converted target nucleic acids using the thermal modulator and the detector.

In another aspect, the present disclosure provides a computer readable media that comprises non-transitory computer-executable instructions which, when executed by at least one electronic processor, perform at least: performing a melting curve analysis using a thermal modulator and multiple probe sets under conditions sufficient to generate a melting temperature (Tₘ) detection barcode data set from partitioned sample aliquots created from the nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise: at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or, at least two mediator probe nucleic acids and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and, identifying at least two different Tₘ detection barcodes in the Tₘ detection barcode data set using a detector.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of determining a fractional abundance of a target variant in a population according to some aspects disclosed herein.

FIG. 2 is a schematic diagram of an exemplary system suitable for use with certain embodiments.

FIG. 3A-3C. Melting temperature barcoding for target discrimination. (A) Highly multiplexed technique is demanded in a variety of field including pathogen detection, cancer diagnostics, or more specifically, protein and nucleic acids detection. The most commonly used melting temperature barcoding techniques involve the use of (B) TaqMan probe and (C) molecular beacon.

FIGS. 4A and 4B. Barcoding a single target with multiple probes can significantly expand the level of multiplexing. In this example, we use two fluorescence channels and three melting temperatures for barcoding. (A) The traditional method, which employs one melting curve per target, can only distinguish six targets either in bulk or on a digital microfluidic platform. However, by utilizing two different probes (equivalent to having two melting curves or melting temperatures) that target the same primary target (B), the multiplexing capability in bulk remains at six due to the overlapping melting profiles. Nevertheless, at the digital level, it becomes possible to distinguish 15 distinct targets.

FIG. 5. Probe melt on bulk. Experimentally, at least six probe melt curves can be accommodated in a fluorophore channel. HEX and ROX fluorophores are used as examples.

FIG. 6. TaqMan probe melt on bulk. Target 1/KRAS is characterized by a Tm of 63.5° C. in the green channel. Target 2/TP53 Exon 7 is characterized by a Tₘ of 62° C. in the green channel and a Tₘ of 63.5° C. in the red channel. Target 3/BRAF is characterized by a Tₘ of 62.5° C. in the red channel. Three targets cannot be distinguished on bulk without digitization due to the overlapping Tₘ.

FIGS. 7A and 7B. TaqMan probe melt on digital. Three targets that cannot be distinguished on bulk can be differentiated on chip as the overlapping Tm peaks are physically separated in different nanowells. (A) Digital TaqMan probe melt scheme. (B) HEX/green melt peaks for target 1. HEX/green and ROX/red melt peaks for target 2. ROX melt peaks for target 3.

FIGS. 8A and 8B. Mediator probe melt on bulk. (A) All six targets can be differentiated by their own melt profile signatures when separated into different wells. Target 1 has a Tₘ of 65° C. in the green channel. Target 2 is characterized by the coexistence of a melt peak with a Tₘ of 61° C. in the red channel and a melt peak with a Tm of 65° C. in the green channel. Target 3 has a Tm of 61° C. in the red channel. Target 4 has a Tₘ of 64° C. in the red channel. Target 5 has a Tₘ of 59.5° C. I the green channel. Target 6 is characterized by the coexistence of a melt peak with a Tₘ of 63.5° C. in the red channel and a melt peak with a Tm of 59.5° C. in the green channel. (B) Six targets cannot be differentiated due to the overlapped Tₘ profiles.

FIGS. 9A and 9B. Mediator probe melt on digital. (A) Digital mediator probe melt scheme. (B) Six targets that cannot be distinguished on bulk can be differentiated on chip as the overlapping Tm peaks are physically separated in different nanowells.

FIG. 10. Protein detection with PEA. In homogenous PEA assay, a mixture of PEA probes are introduced into the sample to recognize specific biomarkers. When two PEA probes bind onto different epitopes on a same protein, the oligo tails are brought into close proximity, hybridize to one another, and then being extended by extension polymerase to generate dsDNA. The immuno-complexes containing DNAs are then digitalized into microarray to perform digital mediator PCR and melt curve analysis, in which each biomarker can be quantified by counting the number of positive wells and identified by specific melt profile.

FIGS. 11A-11C. Microfluidic device design and operation. (A) The microfluidic device is composed of four primary layers: a PDMS-coated glass slide, an ultra-thin PDMS layer, a patterned PDMS layer and a thin glass coverslip between adapters accommodating inlets and outlets. (b) Each device comprises four independent modules, and each module holds 10,040 nanowells. (c) Samples are rapidly drawn into the device, which has been desiccated to create a negative pressure differential. A partitioning liquid injected into the inlet, which is maintained under pressure to lock the digitized sample in place.

FIGS. 12A and 12B. Multicolor imaging system overview. (A) Multicolor system contains imaging components that allow for four-channels imaging and a flatbed heater for thermocycling and melt analysis. (B) CAD design rendering.

FIG. 13. Digital probe melt analysis.

FIGS. 14A-14C. Overview MPM-dMSP. (A) PCR mixture is loaded onto the microfluidic chip and nucleic acids in the sample are digitized. (B) During dMSP, the 5′ flap sequence of mediator probes annealed to the target sequence will be cleaved into mediator primers. Each mediator primer will hybridize to a different position on the corresponding molecular beacon. After enzymatic elongation, the resulting double-stranded reporter will generate a unique fluorescent color-Tm signature for each target. (C) Benchtop melt derivatives of eight targets were acquired in two color channels. Probe melting temperatures ranged from 52.5° C. to 70.5° C.

FIGS. 15A-15C. Analytical validation. (A) Multicolor thermal system setup. (B) Fluorescence images after performing MPM-dMSP for four concentrations of synthetic DNA are shown. (C) Detected versus expected DNA copy number.

FIGS. 16A and 16B. Digital probe melting analysis. (A) Probe melt images are acquired at each temperature increment. (B) Representative melt peak curves and histograms from the red channel demonstrate the multiplexing and quantification capacities of our assay.

FIGS. 17A-17C. FLAP-Melt Overview. (A) DNA is extracted from a liquid biopsy specimen and subjected to bisulfite conversion. The bisulfite-treated DNA is then added to a PCR mixture containing primers and probes and partitioned into nanowells on a microfluidic chip. The chip is placed on a flatbed heater for thermal cycling, followed by probe-based melt curve analysis. (B) During MSP, a flap-labeled probe hybridizes to its target sequence, leading to flap cleavage as Taq polymerase extends the primer. The cleaved flap then binds to its corresponding universal reporter and is extended by polymerase, generating a fluorescence signal. During subsequent melt curve analysis, the duplex of the universal probe and extended flap denatures, causing a fluorescence drop as the temperature increases. A combination of fluorescence color and melting temperature (Tₘ) encodes for target identity. (C) Example module on a microfluidic chip used for melt curve data analysis. Target classification is performed using a support vector machine.

FIGS. 18A-18D. Combinatorial encoding scheme for scalable multiplexing potential. (A) Schematic for color-Tm coding with multiple colors and multiple Tₘ(s) for a single target. (B) Theoretical calculation of the multiplexing potential with N colors and M Tₘ codes. (C) Experimental scheme. Mixture of universal reporters and flaps are spiked into the digital device. The multiplex readout composes of an average of 600 melt curves in each color, with standard deviation represented in the shaded area. (D) Representative melt curves from ROX channel with resolvable Tₘ(s).

FIGS. 19A-19D. Melting temperature resolution determination. (A) Representative melt images of four distinct flaps in the ROX channel. (B) Probability density function of melt derivatives for 12 representative flaps in the ROX channel, with the 99% confidence interval shown as a shaded area. (C) Statistical analysis of melt derivatives for 12 representative flaps in the ROX channel. (D) Theoretical multiplexing capacity of FLAP-Melt as a function of fluorescence color channels (N), compared to commercial digital and real-time PCR systems.

FIG. 20. 138 unique labeling signatures. Different types of combinations are included: single-Tm-single-color, single-Tm-multiple-color, multiple-Tm-single-color, and multiple-Tm-multiple-color combinations.

FIGS. 21A-21E. Development of the 15-plex FLAP-Melt assay. (A) Representative fluorescence micrographs of the chip, illustrating multiplexed detection of five targets in the ROX channel. (B) Bulk melt derivative curves of the 15-plex FLAP-Melt assay. (C) Representative digital melt derivative curves of the 15-plex FLAP-Melt assay. (D) Statistical analysis of digital melting derivative data. (E) Confusion matrices for three support vector machines trained for target classification in each of the three-color channels, where diagonal values indicate correct classifications and off-diagonal values represent misclassifications.

FIG. 22. Analytical validation was performed using synthetic DNA equivalents of bisulfite-converted target loci at copy numbers of 0, 12.5, 25, 50, 100, and 200, each mixed with 18 ng of unmethylated genomic background DNA. Each experiment was performed in triplicate to test the reproducibility of each assay.

FIG. 23. Analytical validation was performed using bisulfite-converted genomic methylated DNA at copy numbers of 0, 25, 50, or 100. The same amount of unmethylated genomic background DNA (18 ng) was employed here. Each experiment was performed in triplicate to test the reproducibility of each assay.

FIGS. 24A-24D. Methylation analysis in Aza-treated cell lines. Normalized methylation levels in untreated and 500 nM Aza-treated cells for H23 (A) and H520 (B) cell lines. Relative methylation changes in Aza-treated cells, normalized to untreated controls, are shown as clustered bar plots. Each cluster represents a loci from the 15-plex panel, with bars indicating different time points. (C) H23 cell line. (D) H520 cell line.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, computer readable media, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Amplifying: As used herein, “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using RT-PCR is a form of amplification. Furthermore, the generation of multiple RNA molecules from a single DNA molecule during the process of transcription is also a form of amplification.

Detect: As used herein, “detect,” “detecting,” or “detection” refers to an act of determining the existence or presence of one or more target nucleic acids (e.g., nucleic acids having targeted mutations or other markers) or other analytes in a sample.

Detectable Signal: As used herein, “detectable signal” refers to signal output at an intensity or power sufficient to be detected in a given detection system. In certain embodiments, a detectable signal is emitted from a label (e.g., a fluorescent label or the like) associated with a given primer nucleic acid and/or probe nucleic acid.

Exonuclease Probe: As used herein, “exonuclease probe” refers to a labeled oligonucleotide that is capable of producing a detectable signal change upon being cleaved. To illustrate, in certain embodiments an exonuclease probe is a 5′-nuclease probe comprising two labeling moieties and emits radiation of increased intensity after one of the labels is cleaved or otherwise separated from the oligonucleotide. In some of these embodiments, for example, 5′-nuclease probe is labeled with a 5′ terminus quencher moiety and a reporter moiety at the 3′ terminus of the probe. In certain embodiments, 5′-nuclease probes are labeled at one or more positions other than, or in addition to, these terminal positions. When the probe is intact, energy transfer typically occurs between the labeling moieties such that the quencher moiety at least in part quenches the fluorescent emission from the acceptor moiety. During an extension step of a polymerase chain reaction, for example, a 5′-nuclease probe bound to a template nucleic acid is cleaved by 5′ to 3′ nuclease activity of, e.g., a Taq polymerase or another polymerase having this activity such that the fluorescent emission from the acceptor moiety is no longer quenched. To further illustrate, in certain embodiments 5′-nuclease probes include regions of self-complementarity such that the probes are capable of forming hairpin structures under selected conditions. In these embodiments, 5′-nuclease probes are also referred to herein as “hairpin probes.”

Hairpin Probe: As used herein, “hairpin probe” refers to an oligonucleotide that can be used to effect target nucleic acid detection and that includes at least one region of self-complementarity such that the probe is capable of forming a hairpin or loop structure under selected conditions. Typically, hairpin probes include one or more labeling moieties. In one exemplary embodiment, quencher moieties and reporter moieties are positioned relative to one another in the hairpin probes such that the quencher moieties at least partially quench light emissions from the reporter moieties when the probes are in hairpin confirmations. In contrast, when the probes in these embodiments are not in hairpin confirmations (e.g., when the probes are hybridized with target nucleic acids), light emissions the acceptor reporter moieties are generally detectable. Hairpin probes are also known as molecular beacons in some of these embodiments. Hairpin probes can also function as 5′-nuclease probes or hybridization probes in certain embodiments.

Hybridization Probe: As used herein, “hybridization probe” refers an oligonucleotide that includes at least one labeling moiety that can be used to effect target nucleic acid detection. In some embodiments, hybridization probes function in pairs. In some of these embodiments, for example, a first hybridization probe of a pair includes at least one donor moiety at or proximal to its 3′-end, while the second hybridization probe of the pair includes at least one acceptor moiety (e.g., LC-Red 610, LC-Red 640, LC-Red 670, LC-Red 705, JA-270, CY5, or CY5.5) at or proximal to its 5′-end. The probes are typically designed such that when both probes hybridize with a target or template nucleic acid (e.g., during a PCR), the first hybridization probe binds to 5′-end side or upstream from the second hybridization probe and within sufficient proximity for energy transfer to occur between the donor and acceptor moieties to thereby produce a detectable signal. Typically, the second hybridization probe also includes a phosphate or other group on its 3′-end to prevent extension of the probe during a PCR.

Label: As used herein, “label” refers to a moiety attached (covalently or non-covalently), or capable of being attached, to a molecule, which moiety provides or is capable of providing information about the molecule (e.g., descriptive, identifying, etc. information about the molecule). Exemplary labels include donor moieties, acceptor moieties, fluorescent labels, non-fluorescent labels, calorimetric labels, chemiluminescent labels, bioluminescent labels, radioactive labels, mass-modifying groups, antibodies, antigens, biotin, haptens, and enzymes (including, e.g., peroxidase, phosphatase, etc.).

Mediator Probe Nucleic Acid: As used herein, “mediator probe nucleic acid” refers to a probe nucleic acid that comprises a first subsequence that binds to a given target nucleic acid and a second subsequence that binds to a reporter probe nucleic acid when cleaved from the mediator probe nucleic acid.

Melting Temperature Detection Barcode: As used herein, a “melting temperature detection barcode” or “Tₘ detection barcode” refers one or more melting temperatures and/or one or more labels of one or more probe nucleic acids in a specified probe set that differentiate a specified target nucleic acid from other detected nucleic acids in a sample when the one or more probe nucleic acids dissociate from the specified target nucleic acid, for example, as part of a given melting curve analysis.

Mixture: As used herein, “mixture” refers to a combination of two or more different components.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Primer Nucleic Acid: As used herein, “primer nucleic acid” or “primer” refers to a nucleic acid that can hybridize to a target or template nucleic acid and permit chain extension or elongation using, e.g., a nucleotide incorporating biocatalyst, such as a polymerase under appropriate reaction conditions. A primer nucleic acid is typically a natural or synthetic oligonucleotide (e.g., a single-stranded oligodeoxyribonucleotide). Although other primer nucleic acid lengths are optionally utilized, they typically comprise hybridizing regions that range from about 8 to about 100 nucleotides in length. Short primer nucleic acids generally require cooler temperatures to form sufficiently stable hybrid complexes with template nucleic acids. A primer nucleic acid that is at least partially complementary to a subsequence of a template nucleic acid is typically sufficient to hybridize with the template for extension to occur. A primer nucleic acid can be labeled, if desired, by incorporating a label detectable by, e.g., spectroscopic, photochemical, biochemical, immunochemical, chemical, or other techniques. To illustrate, useful labels include donor moieties, acceptor moieties, quencher moieties, radioisotopes, electron-dense reagents, enzymes (as commonly used in performing ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

Probe Nucleic Acid: As used herein, “probe nucleic acid” or “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using a probe under sufficiently stringent hybridization conditions permits the selective detection of a specific target sequence. The term “hybridizing region” refers to that region of a nucleic acid that is exactly or substantially complementary to, and therefore capable of hybridizing to, the target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support. A probe of the invention is generally included in a nucleic acid that comprises one or more labels (e.g., donor moieties, acceptor moieties, and/or quencher moieties), such as an exonuclease probe (e.g., a 5′-nuclease probe), a mediator probe nucleic acid, a reporter probe nucleic acid, a proximal binding probe, a hybridization probe, a fluorescent resonance energy transfer (FRET) probe, a hairpin probe, or a molecular beacon, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable hybridization complexes may contain mismatched bases or unmatched bases. Modification of the stringent conditions may be necessary to permit a stable hybridization complex with one or more base pair mismatches or unmatched bases. Stability of the target/probe hybridization complex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids.

Protein: As used herein, “protein” or “polypeptide” refers to a polymer of at least two amino acids attached to one another by a peptide bond. Examples of proteins include enzymes, hormones, antibodies, and fragments thereof.

Proximal Binding Probe Pair. As used herein, “proximal binding probe pair” refers to a pair of probes that include a first binding probe having a first target protein binding moiety (e.g., an antibody or a protein binding portion thereof) coupled to a first oligonucleotide that includes a first hybridization site and a second binding probe having a second target protein binding moiety coupled to a second oligonucleotide that includes a second hybridization site. The first and second target protein binding moieties bind to different epitopes on a given target protein and the first and second hybridization sites hybridize with one another when the first and second target protein binding moieties bind to the different epitopes on the given target protein to produce a given pairwise probe bound target protein. In some embodiments, a proximal binding probe pair includes an oligonucleotide-coupled antibody probe pair.

Reaction Mixture: As used herein, “reaction mixture” refers a mixture that comprises molecules that can participate in and/or facilitate a given reaction or assay. To illustrate, an amplification reaction mixture generally includes a solution containing reagents necessary to carry out an amplification reaction, and typically contains primers, a biocatalyst (e.g., a nucleic acid polymerase, a ligase, etc.), dNTPs, and a divalent metal cation in a suitable buffer. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Reference: As used herein, “reference” in the context of a nucleic acid refers to a known sequence used for purposes of comparison with experimentally determined or test sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, at least about 100000, at least about 1000000, at least about 1000000000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” refers to a tissue or organ from a subject; a cell (either within a subject, taken directly from a subject, or a cell maintained in culture or from a cultured cell line); a ceil lysate (or lysate fraction) or cell extract; or a solution containing one or more molecules derived from a cell or cellular material (e.g., a nucleic acid), which is assayed as described herein. A sample may also be any body fluid or excretion (for example, but not limited to, blood, urine, stool, saliva, tears, bile) that contains cells, cell components, or non-cellular fractions.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a respiratory disease, disorder, or condition, is going to receive a therapy for a respiratory disease, disorder, or condition, and/or has received at least one therapy for a respiratory disease, disorder, or condition.

System: As used herein, “system” in the context of analytical instrumentation refers a group of objects and/or devices that form a network for performing a desired objective.

Target: As used herein, “target” refers to a biomolecule (e.g., a nucleic acid, etc.), or portion thereof, that is to be amplified, detected, and/or otherwise analyzed.

Treatment: As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

Value: As used herein, “value” generally refers to an entry in a dataset that can be anything that characterizes the feature to which the value refers. This includes, without limitation, numbers, words or phrases, symbols (e.g., + or −) or degrees.

### DETAILED DESCRIPTION

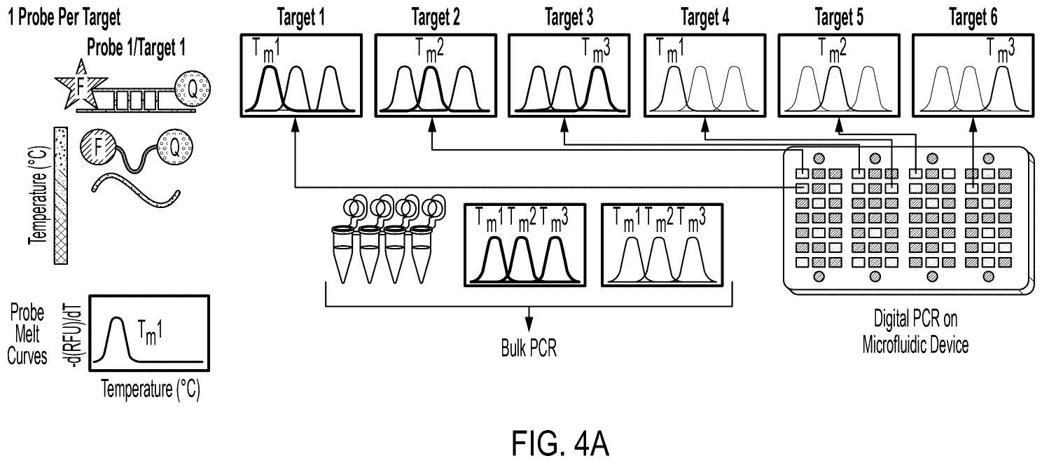

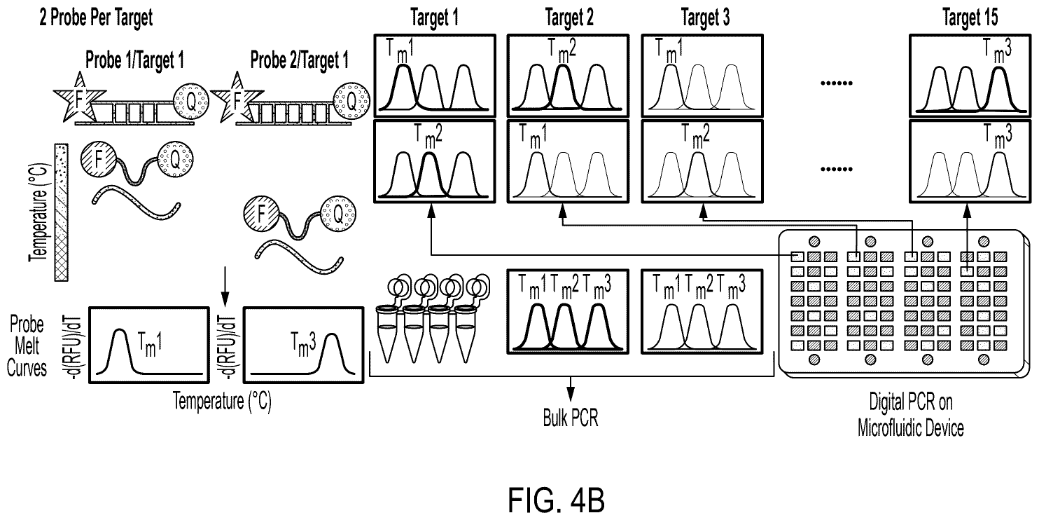

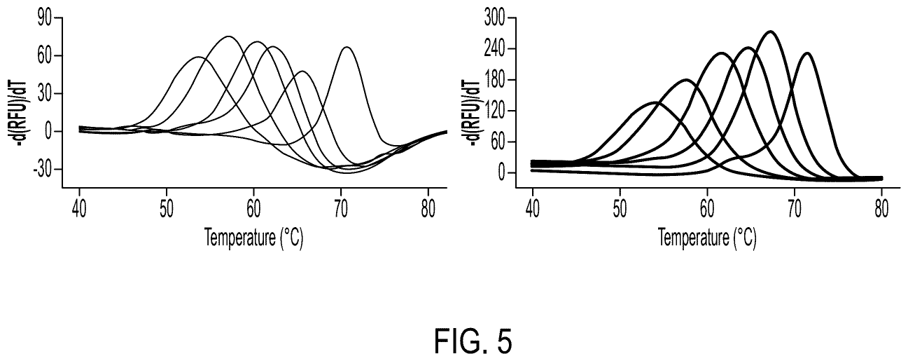

The present disclosure provides for the multiplex PCR-based detection of analytes, such as nucleic acids and proteins. The methods and related aspects of the present disclosure overcome many of the limitations of pre-existing techniques for multiplexed analyte detection, including limitations due to a restricted number of available color channels in the associated instruments. In some embodiments, for example, the present disclosure provides a method called d-3D melt that couples a 3-dimensional melting curve labeling scheme and digital microfluidics to achieve a high-level of multiplexed detection (FIG. 4B). To illustrate, with two fluorescence channels and three melting temperatures in each fluorescence channel, by barcoding each target with two distinct melting curves, after digital PCR on a microfluidic device, 15 targets can be distinguished from one another. The level of multiplexing exponentially increases with an increasing number of fluorophores and an increasing number of melting curves fit in each fluorescence channel. To further illustrate, with 6 melting curves per channel (FIG. 5), if each target is encoded by 2 melting curves, up to 66 targets can be distinguished. These and other attributes of the present disclosure will be apparent upon a complete review of the specification, including the accompanying figures.

### Exemplary Methods

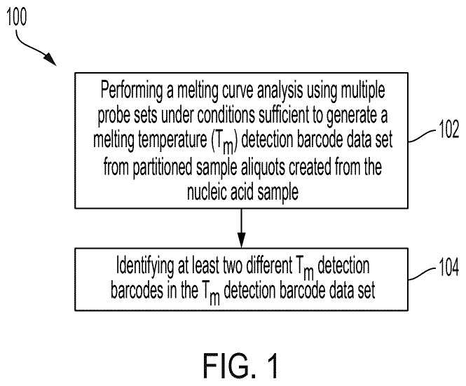

To illustrate, FIG. 1 is a flow chart that schematically shows exemplary method steps of detecting multiple target nucleic acids in a nucleic acid sample. As shown, method 100 includes performing a melting curve analysis using multiple probe sets under conditions sufficient to generate a melting temperature (Tₘ) detection barcode data set from partitioned sample aliquots created from the nucleic acid sample (step 102). In some embodiments, the probe nucleic acids comprise labeled probe nucleic acids (e.g., fluorescently labeled probe nucleic acids). Exemplary labels are described further herein. Typically, a plurality of the partitioned sample aliquots each comprise at most one, if any, target nucleic acid and at least one of the probe sets. A specified Tₘ detection barcode in the Tₘ detection barcode data set generally includes one or more melting temperatures of one or more probe nucleic acids in a specified probe set when the one or more probe nucleic acids dissociate from a specified target nucleic acid. In some embodiments, method 100 includes performing a bulk PCR technique using the nucleic acid sample. In some embodiments, method 100 includes performing an asymmetric PCR technique using the nucleic acid sample. In some embodiments, an emulsion comprises the partitioned sample aliquots. In some embodiments, method 100 includes generating the Tₘ detection barcode data set using a microfluidic device. In some embodiments, method 100 includes performing a digital PCR technique using the nucleic acid sample prior to and/or when identifying the at least two different Tₘ detection barcodes. In some embodiments, the nucleic acid sample comprises one or more bisulfite converted target nucleic acids and method 100 further comprises identifying one or more methylation patterns in the bisulfite converted target nucleic acids. Method 100 also includes identifying at least two different Tₘ detection barcodes in the Tₘ detection barcode data set (step 104). In some embodiments, the method comprises using one or more nucleic acids comprising a nucleotide sequence selected from SEQ ID NOS: 1-30 to generate at least a portion of the Tₘ detection barcode data set.

The multiple probe sets include various embodiments. In some embodiments, for example, the multiple probe sets include at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid. In some embodiments, the multiple probe sets include at least one probe nucleic acid that binds to a given target nucleic acid in the partitioned sample aliquots and that comprises a melting temperature when dissociated from the given target nucleic acid to produce a given Tₘ detection barcode for the given target nucleic acid. In some embodiments, the multiple probe sets include at least one probe set having a single probe nucleic acid that binds to a particular target nucleic acid in the partitioned sample aliquots and that comprises a melting temperature when dissociated from the particular target nucleic acid to produce a particular Tₘ detection barcode for the particular target nucleic acid. In some embodiments, the multiple probe sets comprise exonuclease probes (e.g., 5′-nuclease probes or TaqMan® probes), hairpin probes (e.g., molecular beacons, etc.), and/or hybridization probes. In some embodiments, the multiple probe sets are configured to generate a Tₘ detection barcode data set that comprises at least 3, at least 4, at least 5, at least 6, at least 7, at least 8, at least 9, at least 10, at least 15, at least 25, at least 50, at least 75, at least 100, at least 200, at least 300, at least 400, at least 500, at least 600, at least 700, at least 800, at least 900, at least 1000, or more different Tₘ detection barcodes.

Typically, the probe nucleic acids each comprise at least one fluorescent labeling moiety. Suitable fluorescent labeling moieties are disclosed further herein. In some embodiments, multiple probe nucleic acids in a given probe set comprise an identical fluorescent labeling moiety. In some embodiments, multiple probe nucleic acids in a given probe set comprise a different fluorescent labeling moiety.

In some embodiments, the multiple probe sets include at least two mediator probe nucleic acids and at least one reporter probe nucleic acid. The mediator probe nucleic acids each comprise a first subsequence that binds to a given target nucleic acid in the partitioned sample aliquots and a second subsequence that binds to the reporter probe nucleic acid when cleaved from the mediator probe nucleic acids. Cleaved second subsequences from the at least two mediator probe nucleic acids each comprise different melting temperatures when dissociated from the reporter probe nucleic acid to produce a given Tₘ detection barcode for the given target nucleic acid. In some embodiments, the multiple probe sets include at least one mediator probe nucleic acid and at least one reporter probe nucleic acid. The mediator probe nucleic acid comprises a first subsequence that binds to a given target nucleic acid in the partitioned sample aliquots and a second subsequence that binds to the reporter probe nucleic acid when cleaved from the mediator probe nucleic acids. A cleaved second subsequence from the at least one mediator probe nucleic acid comprises a melting temperature when dissociated from the reporter probe nucleic acid to produce a given Tₘ detection barcode for the given target nucleic acid. In some embodiments, the multiple probe sets comprise at least one probe set having a single mediator probe nucleic acid and the reporter probe nucleic acid. Typically, method 100 includes extending the mediator probes prior to dissociation from the universal reporter probe. In some embodiments, the reporter probe nucleic acid comprises a universal reporter probe nucleic acid. In some embodiments, a given second subsequence bound to the reporter probe nucleic acid is extended prior to being dissociated from the reporter probe nucleic acid to produce the given Tₘ detection barcode for the given target nucleic acid.

In some embodiments, the methods of the present disclosure include generating the nucleic acid sample from a protein sample. In these embodiments, the methods generally include contacting the protein sample with a proximal binding probe set under conditions sufficient to produce a set of pairwise probe bound target proteins. The proximal binding probe set comprises a plurality of proximal binding probe pairs (e.g., oligonucleotide-coupled antibody probes). A given proximal binding probe pair includes a first binding probe comprising a first target protein binding moiety coupled to a first oligonucleotide that comprises a first hybridization site and a second binding probe comprising a second target protein binding moiety coupled to a second oligonucleotide that comprises a second hybridization site. The first and second target protein binding moieties bind to different epitopes on a given target protein and the first and second hybridization sites hybridize with one another when the first and second target protein binding moieties bind to the different epitopes on the given target protein to produce a given pairwise probe bound target protein. In these embodiments, the methods also include extending the first and second oligonucleotides in the set of pairwise probe bound target proteins to produce extended oligonucleotides in the set of pairwise probe bound target proteins. In these embodiments, the methods also typically include separating the extended oligonucleotides from the first and second target protein binding moieties in the set of pairwise probe bound target proteins to thereby generate the nucleic acid sample from the protein sample. In some embodiments, identifying the at least two different Tₘ detection barcodes in the Tₘ data set thereby further detects multiple target proteins in the protein sample.

The multiple target nucleic acids comprise between about 3 and about 1000 different target nucleic acids, between about 4 and about 500 different target nucleic acids, between about 5 and about 100 different target nucleic acids, between about 6 and about 90 different target nucleic acids, between about 7 and about 80 different target nucleic acids, between about 8 and about 70 different target nucleic acids, between about 9 and about 60 different target nucleic acids, between about 10 and about 50 different target nucleic acids, between about 15 and about 40 different target nucleic acids, or between about 20 and about 30 different target nucleic acids.

In some embodiments, the methods include obtaining the nucleic acid sample from a subject. In some embodiments, one or more of the multiple target nucleic acids that were detected identify the subject. In some embodiments, one or more of the multiple target nucleic acids that were detected are associated with at least one disease state in the subject. In some of these embodiments, the methods further include administering at least one therapy to the subject to treat the disease state in the subject. In some embodiments, the multiple target nucleic acids that were detected are from an infectious organism in the subject. In some embodiments, the multiple target nucleic acids that were detected are from nucleic acid variants associated with the disease state in the subject. In some embodiments, the disease state comprises a cancer type.

### Labeling

The oligonucleotides (e.g., primers, probes, etc.) described herein are optionally labeled, e.g., to facilitate subsequent detection. In some embodiments, the nucleic acid synthesis reagents (e.g., phosphoramidite precursors of nucleotides, etc.) are labeled prior to synthesis of the primer or probe nucleic acids. In certain embodiments, labels and nucleotides are directly conjugated to one another (e.g., via single, double, triple or aromatic carbon-carbon bonds, or via carbon-nitrogen bonds, nitrogen-nitrogen bonds, carbon-oxygen bonds, carbon-sulfur bonds, phosphorous-oxygen bonds, phosphorous-nitrogen bonds, etc.). Optionally, a linker attaches the label to a given nucleotide. A wide variety of linkers can be used or adapted for use in conjugating labels and nucleotides. Certain non-limiting illustrations of such linkers are referred to herein.

Essentially any label is optionally utilized to label the nucleotides and nucleosides utilized in the oligonucletides (e.g., primers, probes, etc.) described herein. In some embodiments, for example, the label comprises a fluorescent dye (e.g., a rhodamine dye (e.g., R6G, R110, TAMRA, ROX, etc.), a fluorescein dye (e.g., JOE, VIC, TET, HEX, FAM, etc.), a halofluorescein dye, a cyanine dye (e.g., CY3, CY3.5, CY5, CY5.5, etc.), a BODIPY® dye (e.g., FL, 530/550, TR, TMR, etc.), an ALEXA FLUOR® dye (e.g., 488, 532, 546, 568, 594, 555, 653, 647, 660, 680, etc.), a dichlororhodamine dye, an energy transfer dye (e.g., BIGDYE® v 1 dyes, BIGDYE® v 2 dyes, BIGDYE® v 3 dyes, etc.), Lucifer dyes (e.g., Lucifer yellow, etc.), CASCADE BLUE®, Oregon Green, and the like. Other labels optionally adapted for use in the methods disclosed herein include, e.g., biotin, weakly fluorescent labels (Yin et al. (2003) Appl Environ Microbiol. 69 (7): 3938, Babendure et al. (2003) Anal. Biochem. 317 (1): 1, and Jankowiak et al. (2003) Chem Res Toxicol. 16 (3): 304), non-fluorescent labels, calorimetric labels, chemiluminescent labels (Wilson et al. (2003) Analyst. 128 (5): 480 and Roda et al. (2003) Luminescence 18 (2): 72), Raman labels, electrochemical labels, radioisotope labels, and bioluminescent labels (Kitayama et al. (2003) Photochem Photobiol. 77 (3): 333, Arakawa et al. (2003) Anal. Biochem. 314 (2): 206, and Maeda (2003) J. Pharm. Biomed. Anal. 30 (6): 1725), among many others.

A large variety of linkers are available for linking labels to nucleic acids and will be apparent to one of skill in the art. A linker is generally of a structure that is sterically and electronically suitable for incorporation into a nucleic acid. Linkers optionally include, e.g., ether, thioether, carboxamide, sulfonamide, urea, urethane, hydrazine, or other moieties. To further illustrate, linkers generally include between about one and about 25 nonhydrogen atoms selected from, e.g., C, N, O, P, Si, S, etc., and comprise essentially any combination of, e.g., ether, thioether, amine, ester, carboxamide, sulfonamide, hydrazide bonds and aromatic or heteroaromatic bonds. In some embodiments, for example, a linker comprises a combination of single carbon-carbon bonds and carboxamide or thioether bonds. Although longer linear segments of linkers are optionally utilized, the longest linear segment typically contains between about three to about 15 nonhydrogen atoms, including one or more heteroatoms.

### Reaction Mixtures

The methods disclosed herein optionally utilize various reaction mixtures that can be used in a wide variety of applications, particularly where it is desirable to detect multiple target nucleic acids in a nucleic acid sample. In some embodiments, for example, reaction mixtures are utilized in performing homogeneous amplification/detection assays (e.g., real-time PCR monitoring), or detecting mutations or genotyping nucleic acids. In certain embodiments, multiple primers and/or probes are pooled together in reaction mixtures for use in applications that involve multiplex formats. Many of these applications are described further herein.

In addition to the oligonucleotides (e.g., primers and probes), reaction mixtures also generally include various reagents that are useful in performing, e.g., nucleotide polymerization, nucleic acid amplification and detection reactions (e.g., real-time PCR monitoring or 5′-nuclease assays), and the like. Exemplary types of these other reagents include, e.g., template or target nucleic acids (e.g., obtained or derived from essentially any source), reference nucleic acids, nucleotides, pyrophosphate, light emission modifiers, biocatalysts (e.g., DNA polymerases, RNA polymerases, etc.), buffers, salts, amplicons, glycerol, metal ions (e.g., Mg⁺², etc.), dimethyl sulfoxide (DMSO), poly rA (e.g., as a carrier nucleic acid for low copy number targets), uracil N-glycosylase (UNG) (e.g., to protect against carry-over contamination). In some kinetic PCR-related applications, reaction mixtures also include probes that facilitate the detection of amplification products. Examples of probes used in these processes include, e.g., hybridization probes, exonuclease probes (e.g., 5′-nuclease probes), and/or hairpin probes.

### Exemplary Systems and Computer Readable Media

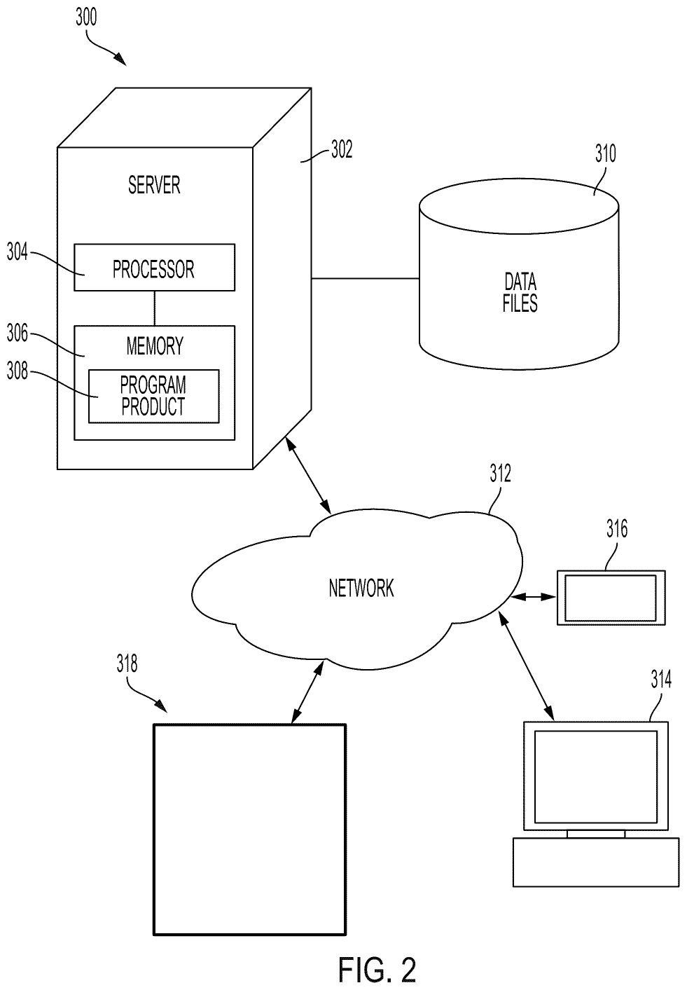

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 300 includes at least one controller or computer, e.g., server 302 (e.g., a search engine server), which includes processor 304 and memory, storage device, or memory component 306, and one or more other communication devices 314, 316, (e.g., client-side computer terminals, telephones, tablets, laptops, other mobile devices, etc. (e.g., for receiving Tₘ detection barcode data sets, etc.)) positioned remote from system components for performing melting curve analysis (e.g., a chamber, a thermal modulator, such as a thermocycler or the like, a detector, etc.) 318, and in communication with the remote server 302, through electronic communication network 312, such as the Internet or other internetwork. Communication devices 314, 316 typically include an electronic display (e.g., an internet enabled computer or the like) in communication with, e.g., server 302 computer over network 312 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 300 also includes program product 308 stored on a computer or machine readable medium, such as, for example, one or more of various types of memory, such as memory 306 of server 302, that is readable by the server 302, to facilitate, for example, a guided search application or other executable by one or more other communication devices, such as 314 (schematically shown as a desktop or personal computer). In some aspects, system 300 optionally also includes at least one database server, such as, for example, server 310 associated with an online website having data stored thereon (e.g., entries corresponding to more reference images, indexed therapies, etc.) searchable either directly or through search engine server 302. System 300 optionally also includes one or more other servers positioned remotely from server 302, each of which are optionally associated with one or more database servers 310 located remotely or located local to each of the other servers. The other servers can beneficially provide service to geographically remote users and enhance geographically distributed operations.

As understood by those of ordinary skill in the art, memory 306 of the server 302 optionally includes volatile and/or nonvolatile memory including, for example, RAM, ROM, and magnetic or optical disks, among others. It is also understood by those of ordinary skill in the art that although illustrated as a single server, the illustrated configuration of server 302 is given only by way of example and that other types of servers or computers configured according to various other methodologies or architectures can also be used. Server 302 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 300. As also understood by those of ordinary skill in the art, other user communication devices 314, 316 in these aspects, for example, can be a laptop, desktop, tablet, personal digital assistant (PDA), cell phone, server, or other types of computers. As known and understood by those of ordinary skill in the art, network 312 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 308 is optionally in the form of microcode, programs, cloud computing format, routines, and/or symbolic languages that provide one or more sets of ordered operations that control the functioning of the hardware and direct its operation. Program product 308, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 608 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 308 is optionally copied from the computer-readable medium to a hard disk or a similar intermediate storage medium. When program product 308, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one captured tissue images and/or the like to be displayed (e.g., via communication devices 314, 316 or the like) and/or receive information from other system components and/or from a system user (e.g., via communication devices 314, 316, or the like).

In some aspects, program product 308 includes non-transitory computer-executable instructions which, when executed by electronic processor 304 perform at least: performing a melting curve analysis using a thermal modulator and multiple probe sets under conditions sufficient to generate a melting temperature (Tₘ) detection barcode data set from partitioned sample aliquots created from the nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise: at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or, at least two mediator probe nucleic acids and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and, identifying at least two different Tₘ detection barcodes in the Tₘ detection barcode data set using a detector.

System 300 also typically includes additional system components (e.g., a chamber, a thermal modulator, such as a thermocycler or the like, a detector, etc.) 318 that are configured to perform various aspects of the methods described herein. In some of these aspects, one or more of these additional system components are positioned remote from and in communication with the remote server 302 through electronic communication network 312, whereas in other aspects, one or more of these additional system components are positioned local, and in communication with server 302 (i.e., in the absence of electronic communication network 312) or directly with, for example, desktop computer 314.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### EXAMPLES

### Example 1: Multi-Temperature Fluorometric Measurement for Multiplex Analyte Detection with Digital PCR Systems

### Results and Discussion

### Overview of Digital 3D-Melt

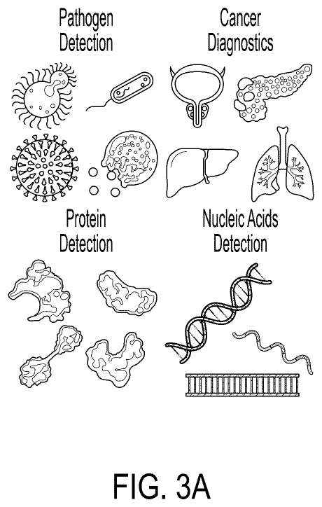

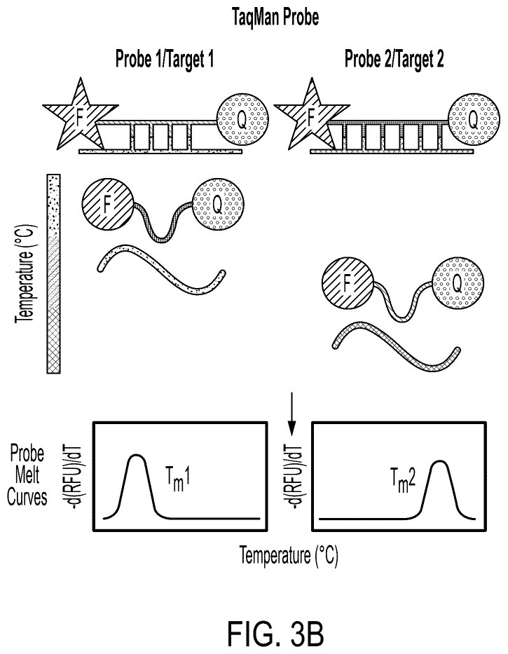

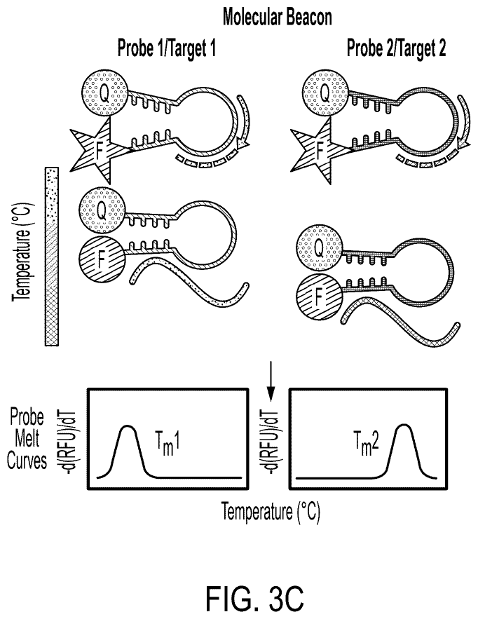

Multiplex detection has vast potential beyond infectious diseases in fields like pan cancer diagnostics and microbiome analysis. Highly multiplexed technology enables simultaneous detection of multiple cancer-associated genetic mutations or protein biomarkers, leading to more efficient and comprehensive cancer profiling. In microbiome research, scalable and multiplexed detection allows for cost-effective analysis of diverse microorganisms, providing insights into complex microbial communities (FIG. 3A). Expanding multiplex detection opens new avenues for diagnostics and surveillance, empowering healthcare decisions, research, and public health policies, revolutionizing various fields and advancing our understanding of complex biological systems.

To enable ultra-highly multiplexed nucleic acid and/or protein detection, we have demonstrated a method called d-3D melt. In some embodiments of this approach, samples are mixed with a reaction mix containing fluorescence probes labeled with different fluorophores, PCR polymerase, and primers. The mixture is loaded onto a microliter-sized array chip, ensuring that each reaction compartment contains no more than one target molecule. Each compartment undergoes PCR amplification and/or extension to generate a sufficient single-stranded template for the fluorescence probes to bind to, resulting in the generation of melt peaks during the subsequent melt curve analysis. At least two methods can be employed for this purpose. One of them includes asymmetric PCR, which uses an excess of one primer compared to the other, leading to the preferential amplification of one DNA strand. This allows for the generation of single-stranded DNA products that can interact with one or more probes, generating melt peaks in different color channels (FIG. 3B).

Another exemplary method is mediator PCR, where the PCR reaction mixture contains tagged PCR primers, universal primers, target-specific probes attached to mediator primers, and universal molecular beacon reporters. Taq polymerase with its 5′ flap endonuclease activity extends the target-specific primers, cleaving the flap sequence of the mediator probes annealed to the target and generating target-specific mediator primers. The universal molecular beacon reporters then capture the corresponding mediator primers and serve as a template for their extension, resulting in target-specific fluorescent colors and probe melting temperatures. Depending on where along the reporter that the mediator primer hybridizes to, the resulting double-stranded reporter will have different length and therefore distinct probe melt Tm (FIG. 3C).

For traditional probe-based melt curve analysis, which exhibits a single melt peak at a single color for each target (FIG. 4). When using the most advanced commercially available digital PCR machine has 6 color channels. Based on the preliminary experiment shown to us, a single-color channel can accommodate at least 6 well-spaced out melt peaks (FIG. 5). Using the traditional probe analysis, a total of 6×6=36 plex detection can be designed, d-3D melt allows for the encoding of a single target with multiple melt peaks in multiple different color channels (FIG. 4). On the other hand, the d-3D melt can achieve up to a hundred thousand based on the calculation of the number of possible color-Tₘ combinations. This capability enhances the multiplexing potential and expands the information obtained from the melt curve analysis.

In this work, we will demonstrate how a single target produces melt peaks in more than one color channel through asymmetric PCR and mediator PCR.

### Taqman Probe Melt Assay Overview & Application in Bulk and in Digital Demonstration

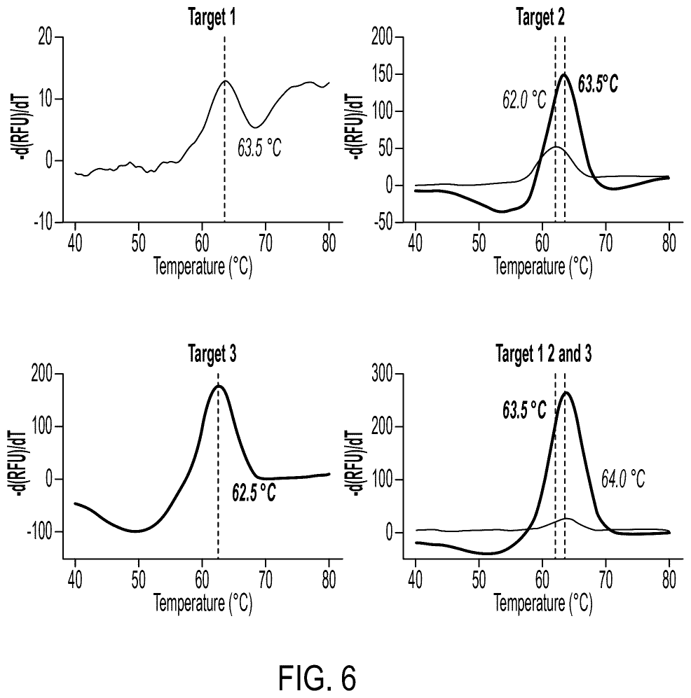

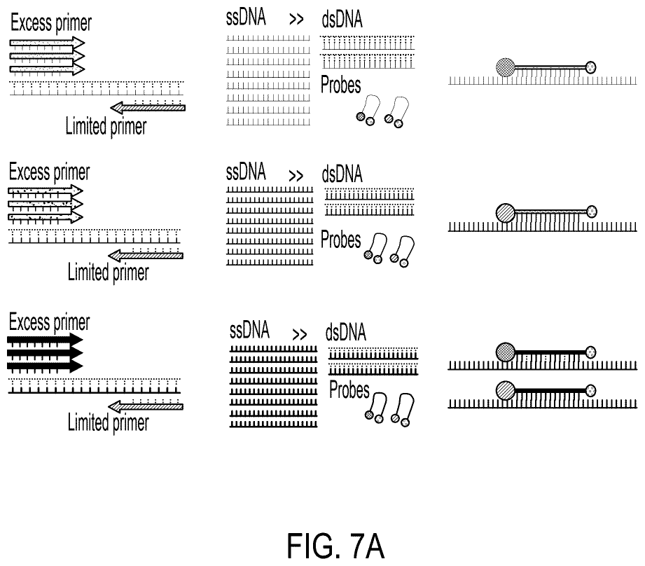

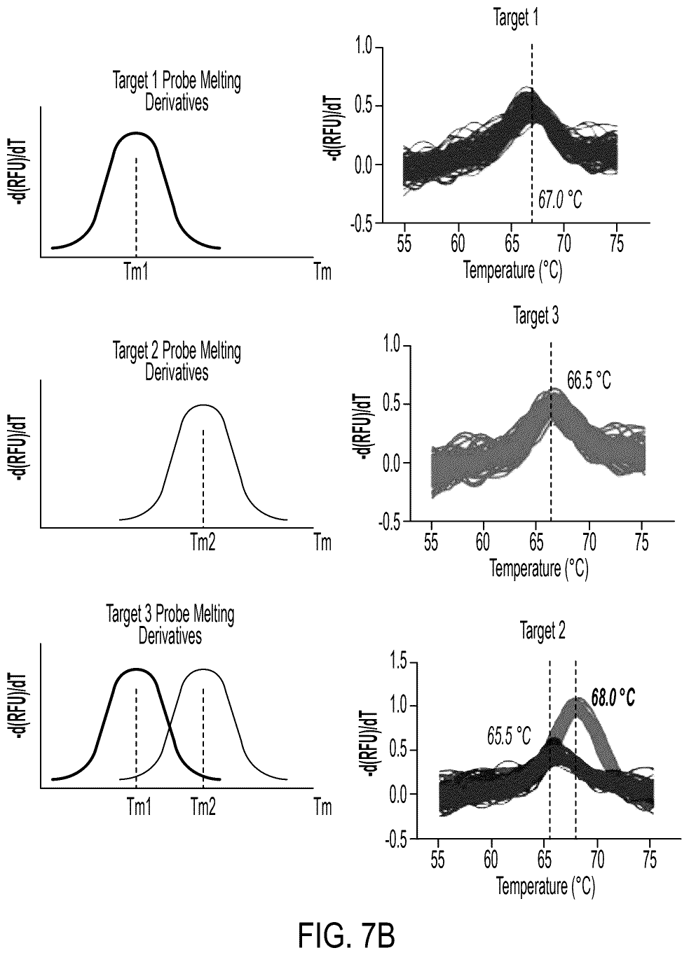

The method, d-3D melt, is compatible with asymmetric PCR for post-PCR probe-based melt curve analysis. In this example, the assay can detect 3 major cancer-relevant nucleic acid mutations-KRAS, Exn7, and BRAF. KRAS is targeted by 1 HEX-labeled probe, Exn7 is targeted by 1 HEX and 1 Texas-Red-labeled probe, and BRAF is targeted by 1 Texas-Red-labeled probe. After asymmetric PCR, Exn7 target can produce melt peak in both HEX and Texas-Red channels, while KRAS showed a single melt peak in HEX channel and BRAF showed a single melt peak in Texas-Red channel (FIGS. 6 and 7). When each target is present in a single reaction, even if two targets produce melt peaks with similar Tₘ at the same color channel, the melt peaks in other color channels can help separate the two targets apart. However, when there is more than 1 target in 1 reaction, the overlap of the melt peak will make the actual composition of the targets in the reaction not identifiable (FIG. 6), and digitization of each targeted gene into individual reactions is necessary. After digitizing the targets, each well only contains a single target, resulting in a melt profile corresponding to each target (FIG. 7).

To enable post-PCR TaqMan probe melting, asymmetric PCR is performed to produce single stranded DNA amplicons, which are further utilized as the targets of TaqMan probes (FIG. 7).

### Mediator Probe Melt Assay Overview and Application (Target Introduction) in Bulk Demonstration (Single Target)

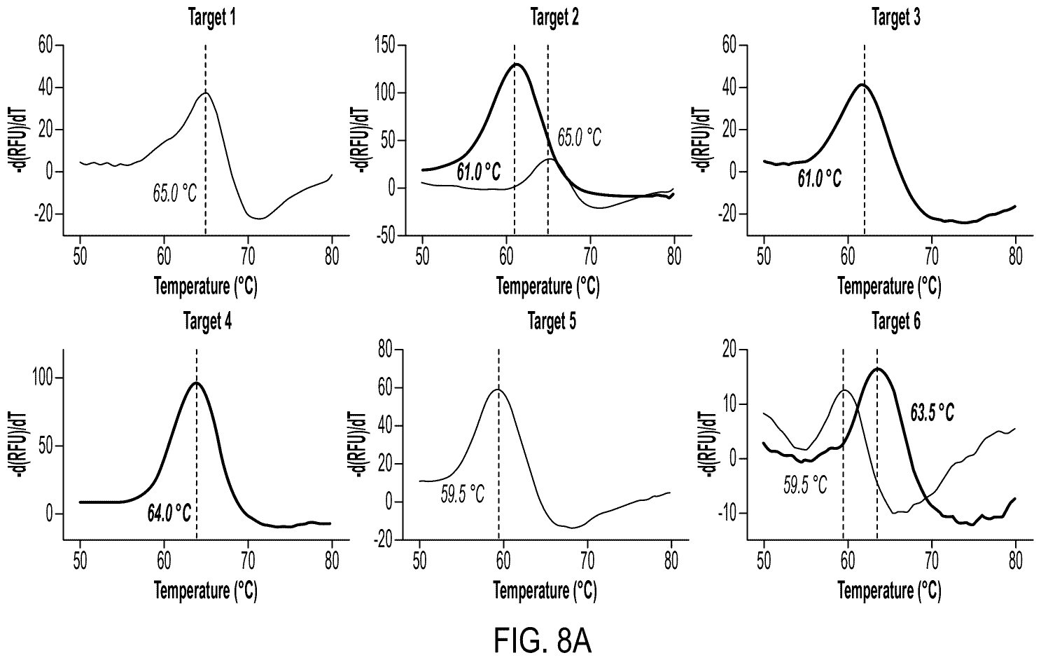

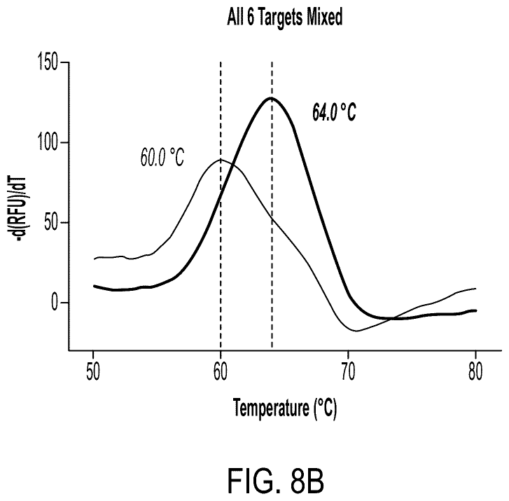

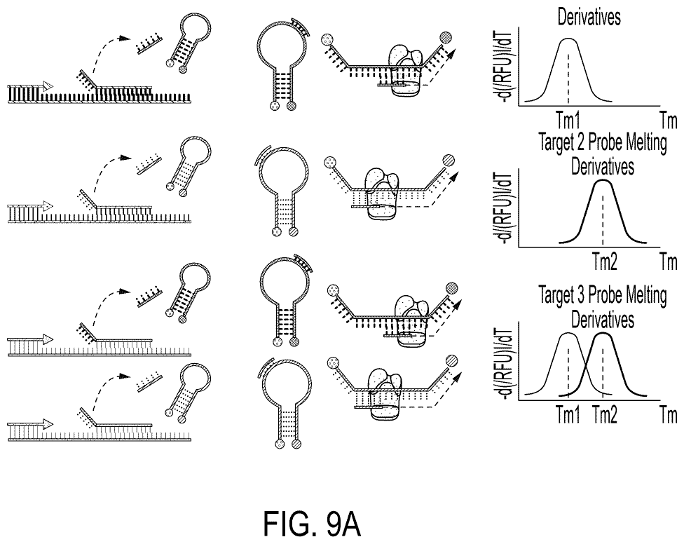

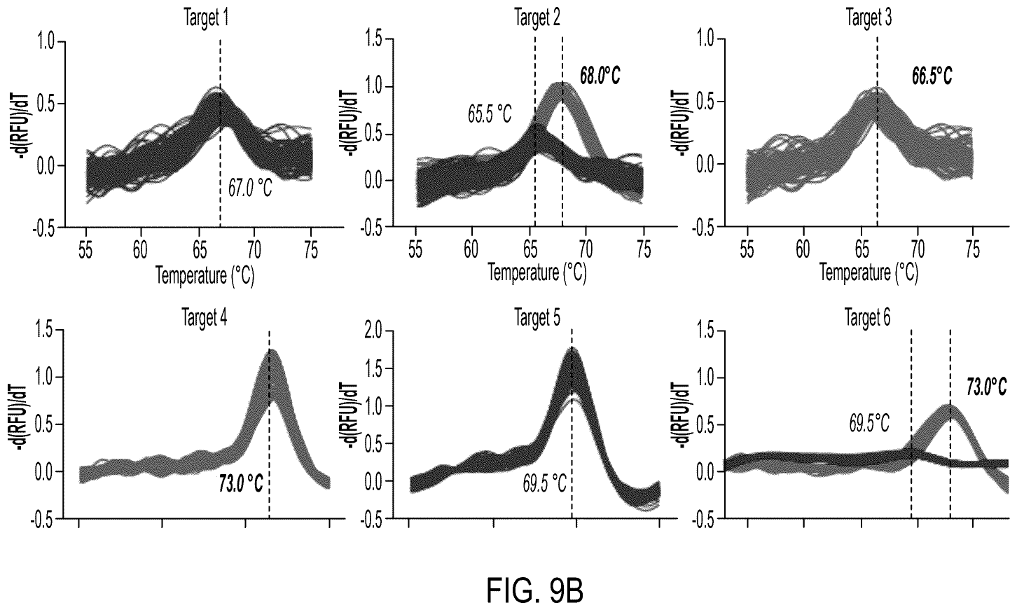

The d-3D Melt is also compatible with mediator probe PCR. This example is demonstrated through a 3-plex assay targeting 3 methylation biomarkers using methylation-specific PCR. Specifically, Target 1 is identified by Tₘ-1 in the HEX channel. Target 2 is identified by the simultaneous presence of Tₘ-1 in the HEX channel and Tₘ-2 in the ROX channel. Lastly, Target 3 is identified by Tₘ-2 in the ROX channel. When each of the targets are present in a single reaction, distinct melt peaks and their corresponding melting temperatures can be used to distinguish each target (FIGS. 8 and 9).

### Mediator Probe Melt in Digital Demonstration (Multiple Targets Per Reaction)

The multiplexing capacity of probe melting can be significantly expanded when the digital platform is employed. As a demonstration, a single PCR reaction mix containing three distinct targets is analyzed both in traditional bulk and digital format. Specifically, target-1 is encoded by fluorophore-1 and Tₘ-1; target-2 is encoded by fluorophore-2 and Tₘ-2; target-3 is encoded by fluorophore-1 at Tₘ1 and fluorophore-2 at Tₘ-2. In the bulk result, differentiation of the three targets is impossible, as only two melt peaks are observed, one for fluorophore-1 at Tₘ-1 and the other for fluorophore-2 at Tₘ-2 (FIG. 8A). Given the simultaneous presence of different targets in the same mixture, encoding targets by redundant fluorophores and Tₘ values failed to provide target identification (FIG. 8B).

Conversely, this limitation is effectively circumvented in the digital platform. When the identical reaction mixture is loaded onto a digital microarray chip, followed by oil partitioning and target digitization, target differentiation becomes viable. Subsequent digital probe melt image analysis yields individual probe melt curve and melt peak for each microchamber (FIG. 9). The microwells, indicated by red curves, with only fluorophore-1 at Tₘ-1 are identified as target-1. The microwells, depicted by green curves, with only fluorophore-2 at Tₘ-2 are identified as target-2. The microwells, represented by yellow curves, with both fluorophore-1 at Tₘ-1 and fluorophore-1 at Tₘ-1 and fluorophore-2 at Tₘ-2 are identified as target-3. These digital results highlight the multiplexing potential inherent to the digital platform. As targets are digitized in separate reaction chambers, each compartment is analyzed individually. This allows for recycling fluorophores and Tₘ values to encode different targets, provided each target maintains a unique fluorophore-Tₘ profile.

Protein Detection with PEA

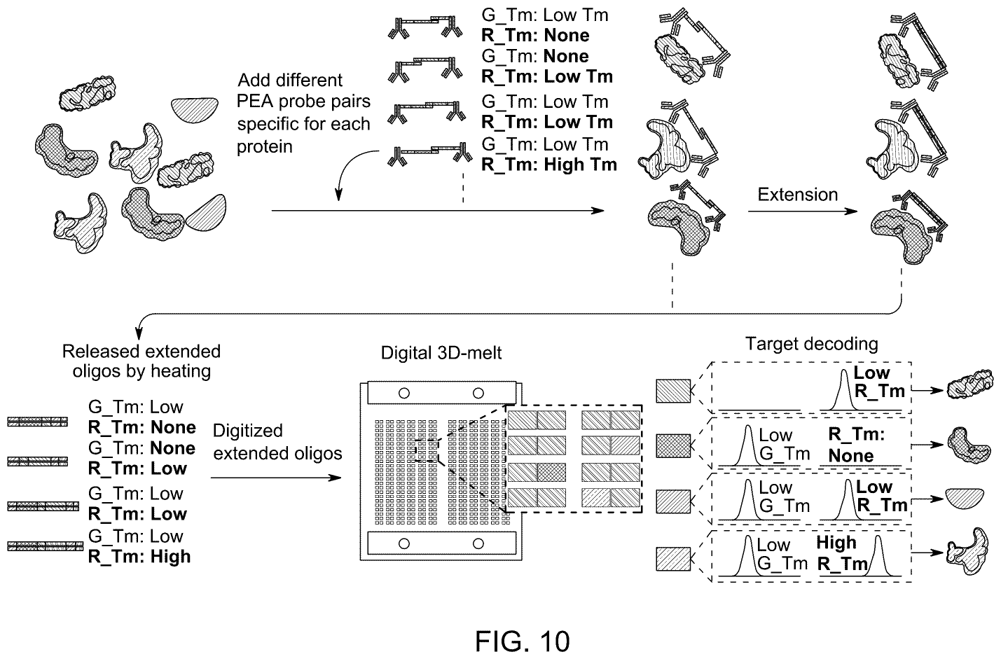

The multi-temperature and fluorometric scheme for highly multiplexed nucleic acid detection can also be adapted for protein analysis by employing homogenous proximity extension assay (PEA). PEA utilizes a pair of antibody-oligo conjugates (thereafter termed as PEA probes) to recognize specific target proteins. When the two PEA probes bind onto different epitopes of the same target, the two oligonucleotides are brought in proximity and the tails are hybridized to each other. In the next step, an extension polymerase is introduced to extend the oligos, resulting in the formation of double-stranded DNA (dsDNA). Subsequently, the immuno-complexes containing DNAs are mixed with PCR buffer and loaded into microarray for mediator PCR amplification and melt analysis, in which each biomarker is digitally quantified and identified by specific melt profile in microwells (FIG. 10). Toward this end, we will first design unique oligo sequences for each biomarker that consist of PCR primer binding sites, TaqMan probe binding sites, and PEA probe hybridization sites. Notably, we will design universal primer binding sites for all the biomarkers tested in a multiplex assay since our multiplexing strategy solely relies on the TaqMan probe melt profiles. This pivotal modification will allow us to use universal primers during PCR amplification, ensuring that all the targets are amplified with minimal bias in efficiency regardless of sequence variations. Next, we will perform antibody-oligo conjugation via Sulfo-SMCC mediated covalent coupling. During the homogenous PEA assay, we will add all the PEA probes for each biomarker into the sample and incubate at 37° C. for 1 hour. When PEA probes bind onto specific biomarkers, we will extend the hybridized oligos and then perform digital PCR and melt for multiplex protein detection.

### Co-Detection of Protein and Nucleic Acid

Our assay can also be implemented to co-detect protein and nucleic acids in one reaction for a given sample. To achieve this, we will first add PEA probes for each biomarker into the sample and incubate at 37° C. for 1 hour to ensure that all biomarkers are tagged with their respective PEA probes. The oligos on PEA probes are then extended by extension polymerase to form dsDNA templates. Next, we will mix the above sample with our mediator PCR buffer, which allows for the amplification of both the nucleic acids and dsDNA on immuno-complexes. The resulting mediator probes after PCR can then undergo probe melt analysis for the simultaneous quantification and identification of proteins and nucleic acids.

### CONCLUSIONS

To achieve highly multiplexed detection of analyte (nucleic acid or protein) without complex instrumentation, the proposed methods provide a highly efficient and low-cost multiplex detection method. By integrating with digital PCR platform, the maximal number of the target nucleic acid or protein that can be detected simultaneously, is not limited by the number of color channels or the number of melt peak per channel.

### Methods

### Taqman Probe Melt Assay Detection on Benchtop

To evaluate the melting temperature profiles among different targets, the reaction was performed in a Bio-Rad CFX96 Touch Real-Time PCR Detection System (Bio-Rad, CA, USA) at 95° C. for 5 s, 30° C. for 5 min, followed by ramping from 30° C. to 95° C. at a 0.5° C. temperature increment between steps and a hold time of 5 s.

### Taqman Probe Melt Assay Composition

A three-plex 10 μL probe-based PCR-melt assay was composed of 0.2 μM TP53 Exon 7 TEX615-labeled probe, 0.2 μM BRAF HEX-labeled probe, 0.2 μM BRAF TEX615-labeled probe, 0.2 μM KRAS HEX-labeled probe, 2 μM TP53 Exon 7 forward primer, 0.2 μM TP53 Exon 7 reverse primer, 2 μM BRAF forward primer, 0.2 μM BRAF reverse primer, 1×Aptogen PCR Mix, 0.05% Tween 20, 0.1 mg/ml BSA, 1 μL of target DNA and water.

### Mediator Probe Detection on Benchtop

The reaction undergoes thermal cycling and probe melt analysis in a CFX96 Touch Real-time PCR Detection System (Bio-Rad) and CFX Manager (version 3.1), respectively. Thermal cycling conditions were 95° C. for 5 min, 4 cycles of (95° C. for 18 sec and 63° C. for 18 sec), 46 cycles of (95° C. for 18 sec and 68.5° C. for 18 sec), 33° C. for 30 min, 95° C. for 1 min, 30° C. for 10 min, followed by a melt curve analysis from 45° C. to 80° C. (0.5° C./step).

### Mediator Probe Assay Composition

Each reaction has a final volume of 20 μL consisting of PCR buffer, 0.01 μM tagged PCR primers, 1 μM universal primer, 0. μM target-specific mediator probes, 0.2 μM molecular beacon reporters, 7 mM MgCl₂, 0.2 μM dNTP mix, 1 mg/mL BSA, 0.01% Tween-20, 0.12 U/μL Platinum Taq polymerase, and 1 uL of bisulfite converted DNA.

### Probe Detection on Digital

Prior to sample loading, the microfluidic chip is left in a desiccator for at least 4 hr. The PCR mixture is transferred into a 1 ml syringe (BD Syringe) and punctured into the sealed inlet of the desiccated microfluidic chip through negative pressure difference. Upon sample loading, the partitional fluid (5 g silicon oil and 1 g of 10:1 PDMS mixture) is pressurized into channels to isolate reaction chambers, facilitating sample digitization across 10,400 nanowells. The PCR mixture in the microfluid chip subsequently undergoes thermal cycling on a flatbed heater (Bulldog Bio), with the use of FC-40 oil between the microfluidic chip and the heating surface to ensure thermal contact. Thermal cycling conditions for TaqMan probe melt experiments were 95° C. for 5 min, 50 cycles of (95° C. for 18 sec and 60° C. for 18 sec). Thermal cycling conditions for mediator probe detection experiments were 95° C. for 5 min, 4 cycles of (95° C. for 18 sec and 63° C. for 18 sec), 66 cycles of (95° C. for 18 sec and 68.5° C. for 18 sec), 5 cycles of (33° C. for 30 min, 95° C. for 1 min, 30° C. for 10 min). Next, the chip is removed from the heater and transferred to the digital multicolor detection platform for digital probe melt curve analysis. The chip was secured to the flatbed heater by tape and underwent temperature ramping at a rate of 0.25° C./s by a melt curve analysis from 45° C. to 80° C. Images were captured with 0.8-s exposure.

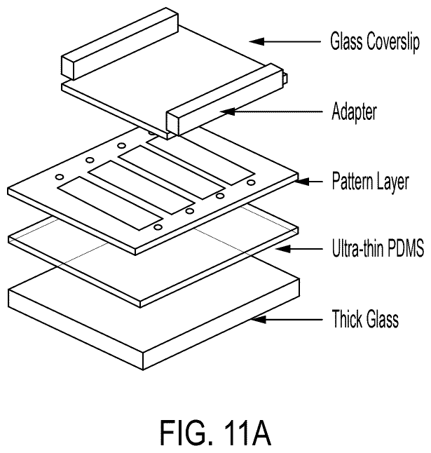

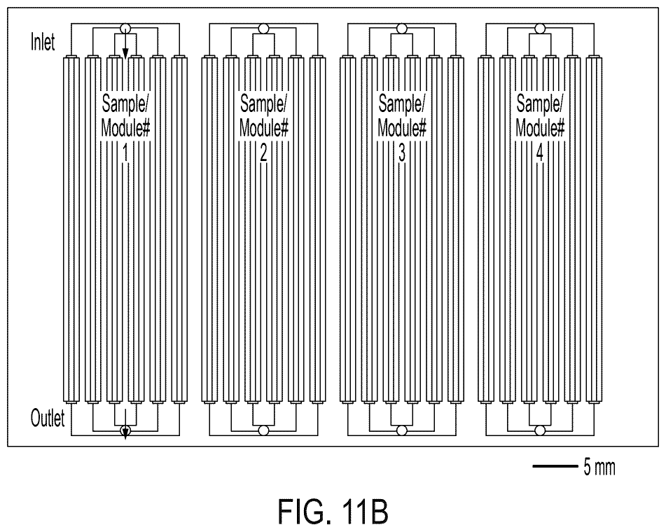

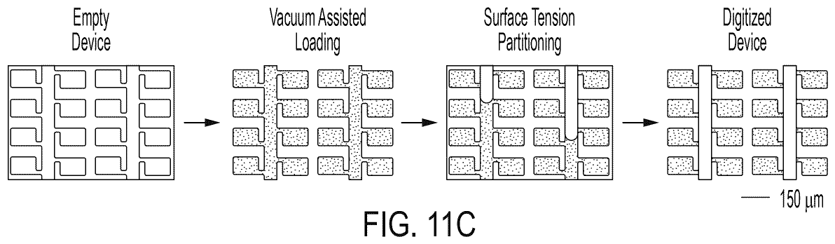

The mdMSP device utilizes a polydimethylsiloxane (PDMS) array fabricated according to a previously-reported ultrathin soft lithography method, which mitigates sample loss during thermocycling and reduces thermal deviation. The device features rapid, vacuum-assisted loading that enables sample loading and digitization to be performed in less than 15 s (FIG. 11). Digitization is achieved through surface-tension-based partitioning by pressure-loading an oil-based solution through the channels. During PCR, partitioning oil remains pressurized to prevent the sample from leaking back into the channels. PDMS was also added to the partitioning oil, which solidifies during PCR to prevent cross-contamination between reaction chambers and allows the device to be readily handled without risk of contamination. In order to increase throughput, we designed a microfluidic device amenable for use with standard fluorescence instrumentation and each chip includes four independent but identical modules, housing 10 040 nanowells each, thereby allowing up to four samples to be digitized and analyzed in parallel (FIG. 11). The nanowells were designed to hold 1 nL of volume, which is small enough to allow absolute digitization of cfDNA template molecules, while retaining physical dimensions compatible with the spatial resolutions of most commercial imaging systems. Imaging was achieved using a flatbed fluorescence scanner, which uses lasers paired with emission filters matched to the respective excitation and emission spectra of each respective fluorophore. The resulting raw imaging data were then processed using a “known mask” mapping technique, as described previously. This customized software program relies on mapping a predefined mask to the array by using four geometric user inputs and then captures the fluorescence intensity value of each well. The entire analysis procedure can be completed in less than 3 min.

### Digital Microarray Device Detection

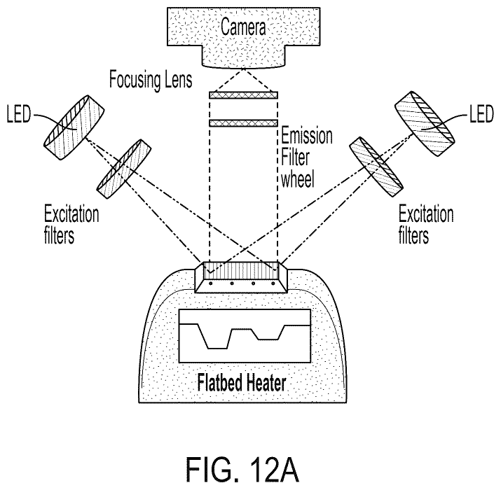

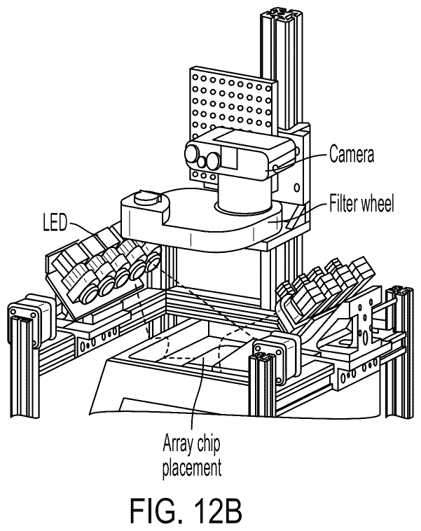

Full-view fluorescent chip images were captured using our in-house built four-color imaging platform (FAM, HEX, Texas Red, and Cy5) (FIG. 12). The setup is composed of a commercial flatbed heater (Bulldog Bio) for thermal control, LED sources (Quadica Development Inc.) with corresponding excitation filters (Omega Optical) for illumination, and a 12M pixel CMOS Mirrorless camera (Sony) with a 50-mm macro focusing lens (Sony) attached to a filter wheel (QHYCCD), which is mounted with emission filters (Omega Optical), for capturing fluorescence images. The imaging platform is controlled with an Arduino microcontroller (Arduino), which is interfaced by a Java-based Graphical User Interface.

### Digital Melt Analysis

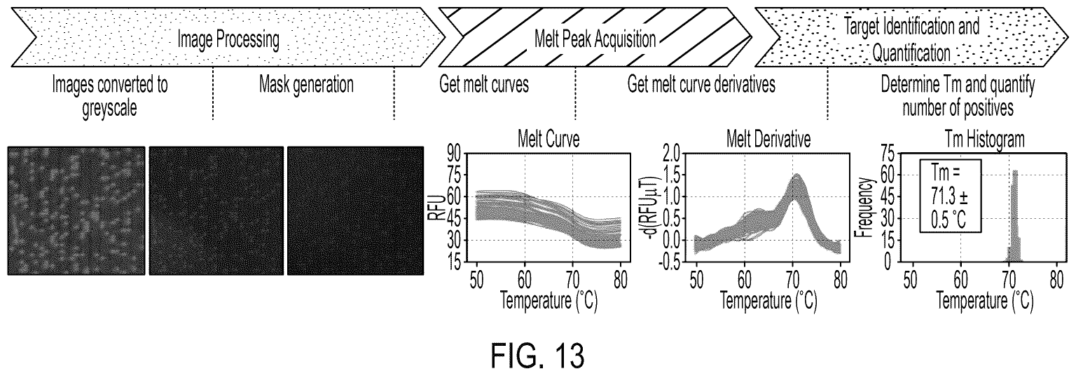

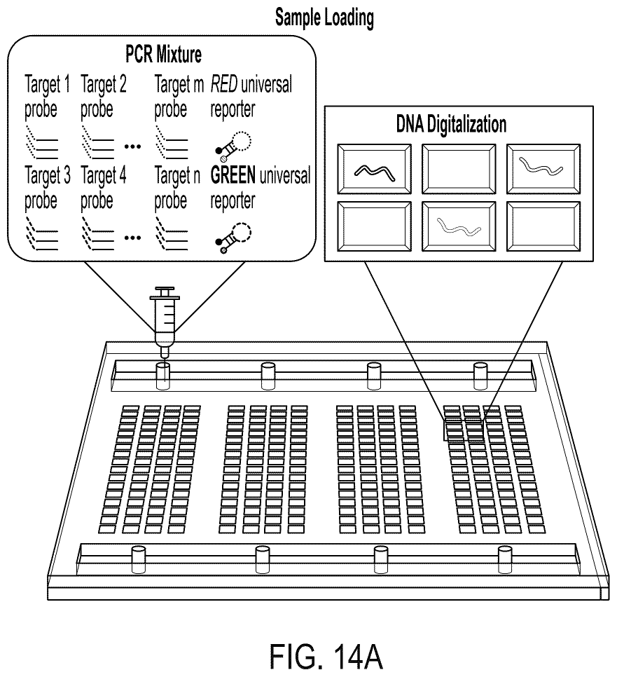

During melting, the chip underwent temperature ramping at a rate of 0.1° C./sec from 45 to 90° C. Data analysis was performed with a custom-developed MATLAB script (FIG. 13). After image collection, the four corners of the chip are selected to generate a virtual mask outlining the expected well locations. Once well locations were determined, the virtual mask was propagated throughout all of the collected images and calculated the signal intensity in each nanowell. Time points were sorted by temperature, and each signal within 0.3° C. was further averaged. A low-pass and Savitzky-Golay filters were used on each nanowell to produce a melt curve. The negative derivative of this signal was taken to obtain a melt peak and further determine the melt temperature of the amplicon in each well.

TABLE 1
Primer and probe for asymmetric PCR experiments.

| Target | Name | Sequence | SEQ ID NO. |
|---|---|---|---|
| KRAS | Forward primer | TCATTATTTTTATTATAAGGCCTGCTGAA | 1 |
| | Reverse primer | CAAAGAATGGTCCTGCACCAGTA | 2 |
| | Probe | /5HEX/TCTTGCCTACGCCACCAGCTCCAACT/3BHQ_1/ | 3 |
| BRAF | Forward primer | TGCTTGCTCTGATAGGAAAATGA | 4 |
| | Reverse primer | CCACAAAATGGATCCAGACAAC | 5 |
| | Probe | /5TEX615/TGGTCTAGCTACAGTGAAATCTC/3IAbRQSp/ | 6 |
| TP53- | Forward primer | GTGGCAAGTGGCTCCTG | 7 |
| Exn7 | Reverse primer | CTCATCTTGGGCCTGTGTTATC | 8 |
| | Probe | /5TEX615/ATGGGCCTCCGGTTCATGTCGCCTAT/3IAbRQSp/ | 9 |
| | Probe | /5HEX/ATGGGCCTCCGGTTCATGTCGCCTAT/3BHQ_1/ | 10 |

TABLE 2
Primer and probe for molecular beacon experiments.

| Target | Name | Sequence | SEQ ID NO. |
|---|---|---|---|
| Target | Forward | AGTTTTTTAATACGGATTTCGTGC | 11 |
| 1 | primer | | |
| | Reverse | CCTCCTAACAAAACCAACGAA | 12 |
| | primer | | |
| | Probe | CCTCTCACACT | 13 |
| | | TGAGGTGT+T+ACGGGGGCGTTGGAGTAT/3AmMO/ | |
| Target | Forward | GGTTTTTACGTTTTTCGTAGTTC | 14 |
| 2 | primer | | |
| | Reverse | CGCTCATCTCCTAAACTCGAA | 15 |
| | primer | | |
| | Probe | CTCTCTCTGG | 16 |
| | | TCGGGATTTGGGGTTGAGTTTTCGTCGC/3AmMO/ | |
| | Probe | CCTCTCACACT | 17 |
| | | TCGGGATTTGGGGTTGAGTTTTCGTCGC/3AmMO/ | |
| Target | Forward | GGTTTTGTGTCGTTCGTTTTC | 18 |
| 3 | primer | | |
| | Reverse | ACGATAATTATACGCCTAAAACTCG | 19 |
| | primer | | |
| | Probe | CTCTCTCTGG TGTGCGCGAGGCGCGGAGTATTT/3AmMO/ | 20 |
| Target | Forward | TTGGTTATAGTCGTTGTTGTTTTTC | 21 |
| 4 | primer | | |
| | Reverse | CCCGAATTTCGATCCCTTCG | 22 |
| | primer | | |
| | Probe | TCTGGTCTATCT | 23 |
| | | TT+T+CGGGGTTTTCGGTTTTCGAGAA/3AmMO/ | |
| Target | Forward | CGAATTTTTATTCGGGAGATCG | 24 |
| 5 | primer | | |
| | Reverse | CCAACCCTCCTTTCAAATATCG | 25 |
| | primer | | |
| | Probe | CACCTCTCAC AAGGCGGGTTGTTTGGTTCGCGAC/3AmMO/ | 26 |
| Target | Forward | GAGTTTAGAAGTTCGGTTGC | 27 |
| 6 | primer | | |
| | Reverse | CCCGACTAAAAAACGCCG | 28 |
| | primer | | |
| | Probe | CACCTCTCAC | 29 |
| | | AGATTCGGGATTTCGGTTTTCGTTTTTCGT/3AmMO/ | |
| | Probe | TCTGGTCTATCT | 30 |
| | | TTCGGGATTTCGGTTTTCGTTTTTCGTTAGT/3AmMO/ | |

### Example 2: Mediator Probe-Based Multiplex Digital Methylation-Specific PCR for Sensitive Detection and Methylation Analysis of Biomarker Panels

Liquid biopsy applications, particularly cell-free tumor DNA analysis, have emerged as non-invasive means of comprehensively and heterogeneously sampling body tissues for cancer diagnostics. DNA methylation is a widely studied cancer detection method. Advances in microfluidics and nucleic acid detection technologies enable digital PCR for liquid biopsy analysis with superior analytical sensitivity and specificity. Although digital methylation-specific PCR (dMSP) is a useful method for detecting DNA methylation, its multiplexing capacity is limited by the number of available fluorophores and cost. In this example, we disclose a platform named mediator probe-based multiplex digital methylation-specific PCR (MPM-dMSP), which integrates a flap endonuclease activity-based high-degree multiplexing method into high-density microfluidic-enabled digital probe melting analysis, for quantitative and simultaneous identification of eight DNA methylation biomarkers.

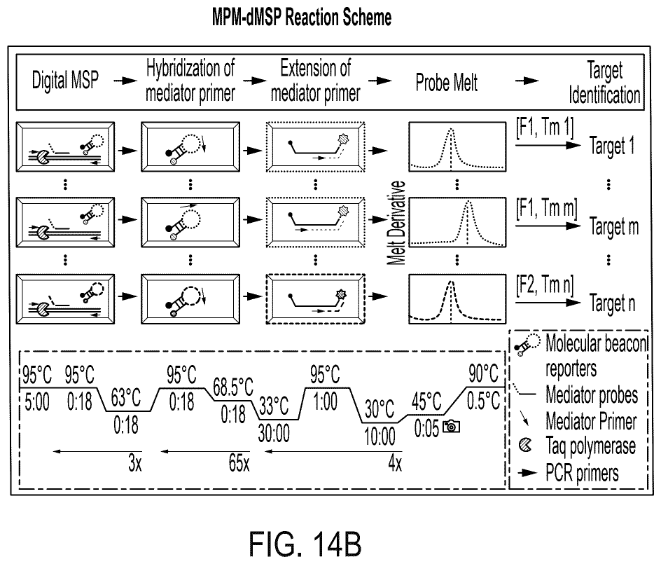

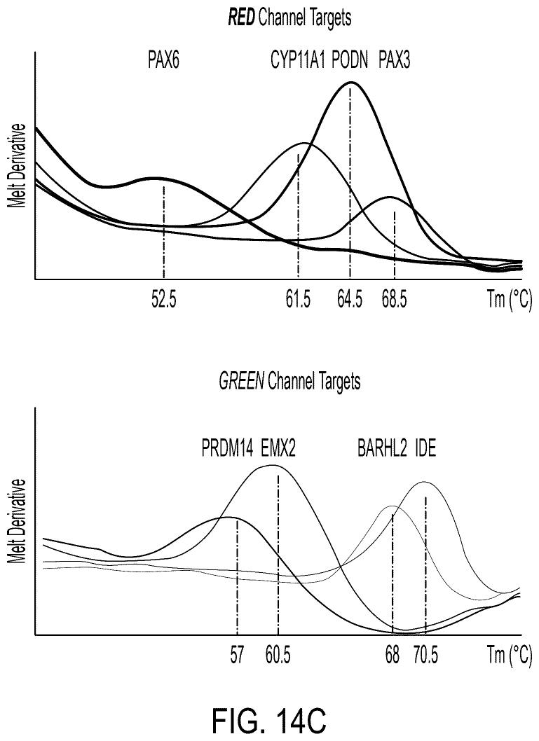

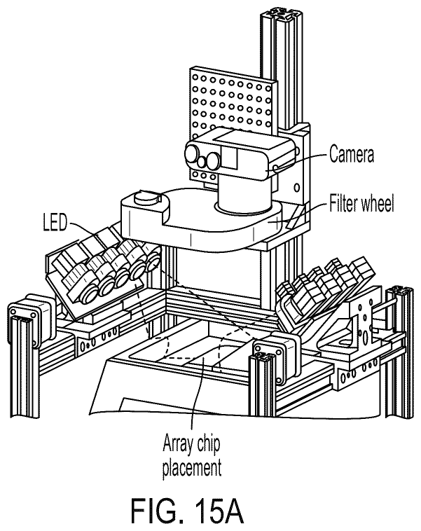

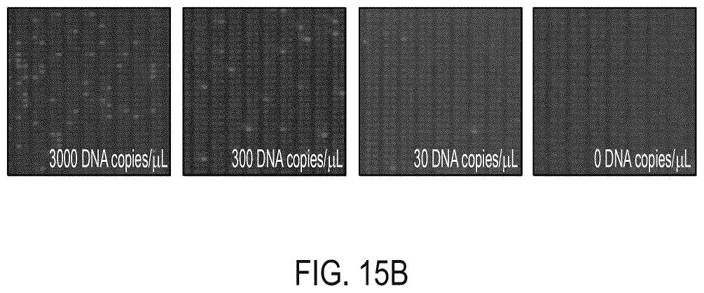

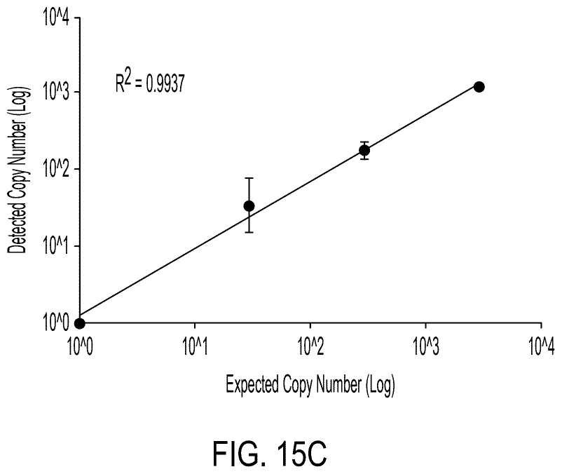

The microfluidic device, consisting of glass and PDMS, is fabricated using ultra-thin soft lithography technique. Each device contains four independent but identical 10,400-nanowell modules. Prior to digital PCR, we vacuum-load the sample into a microfluidic chip and pressure-load partitioning oil such that each nanowell contains no more than one copy of targets (FIG. 14A). The PCR reaction mixture contains eight pairs of tagged PCR primers, universal primer, a cocktail of eight unique mediator primers attached to eight target-specific probes, universal molecular beacon reporters, and Platinum Taq polymerase. MPM-dMSP leverages 5′ flap endonuclease activity of Taq polymerase to first extend the target-specific primers and subsequently cleave the flap sequence of mediator probes annealed to the target sequence, generating target-specific mediator primers (FIG. 14B). Universal molecular beacon reporters then capture the corresponding mediator primers and serve as a template for their extension, producing target-specific fluorescent color and probe melting temperature (FIG. 14C). Homo-Tag Assisted Non-Dimer System (HANDS) reduces primer-dimer formation. For data acquisition, we employed a custom-built multicolor thermal system, consisting of fluorescence imaging components and a flatbed heater (FIG. 15A), to perform thermal cycling and digital melting. Acquired melt images are analyzed in MATLAB.

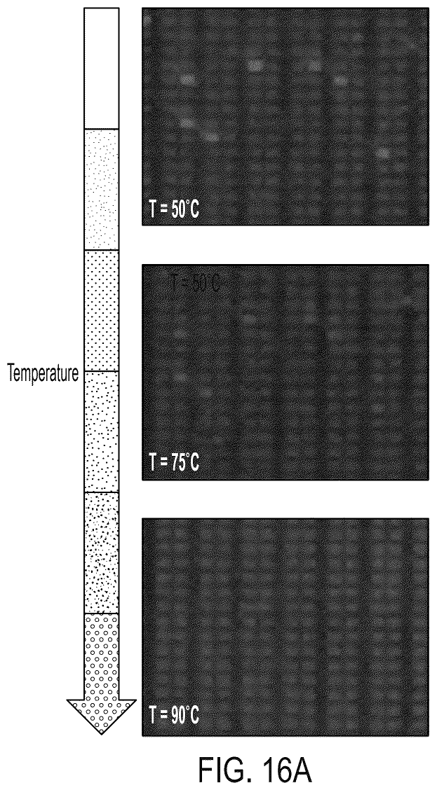

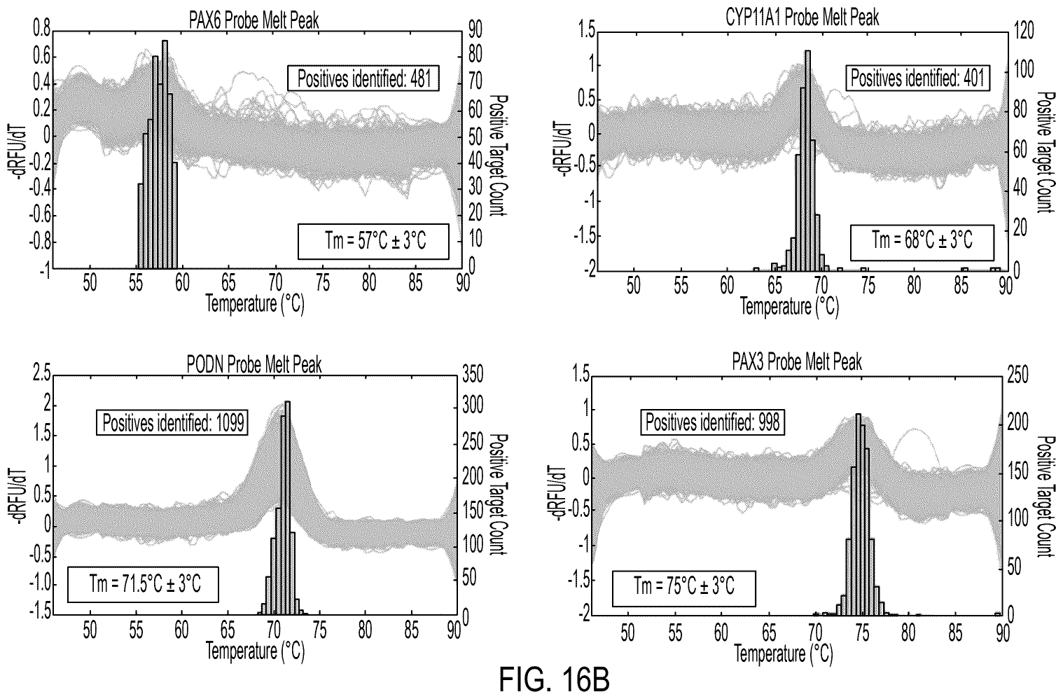

We analytically validated MPM-dMSP by spiking in a known number of synthetic targets representative of bisulfite converted fully methylated sequences of eight genes in a biomarker panel of interest (FIG. 15B). MPM-dMSP demonstrated single-copy sensitivities as the detected and expected numbers of positive wells exhibit a strong linear relationship (FIG. 15C). Digital probe melting analysis, based on extracted fluorescent intensities within nanowells, enables accurate identification of the biomarkers (FIG. 16A). We demonstrated the multiplexing potential of MPM-dMSP by simultaneously detecting eight targets with only two fluorescent channels. Multiplex target detection by a single universal reporter significantly reduces assay complexity as well as time and labor costs. Representative results for four biomarkers in the red channel are shown (FIG. 16B).

In conclusion, this example presents an MPM-dMSP platform, which combines dMSP with flap endonuclease activity-based high-degree multiplexing and microfluidic-based digital probe melting analysis. We showed the multiplex discrimination abilities of MPM-dMSP by detecting a panel of eight epigenetic biomarkers at single-copy sensitivities. Overall, MPM-dMSP provides a simple method for quantitative and simultaneous determination of DNA methylation patterns for biomarker panels.

### Example 2: FLAP-Melt: A Low-Cost High-Throughput Methylation Profiling Through a Highly Multiplexed FLap-Assisted Digital Methylation-Specific PCR-Melt Multi-Temperature Fluorometric Measurement for Multiplex Analyte Detection with Digital PCR Systems

### Overview of FLAP-Melt

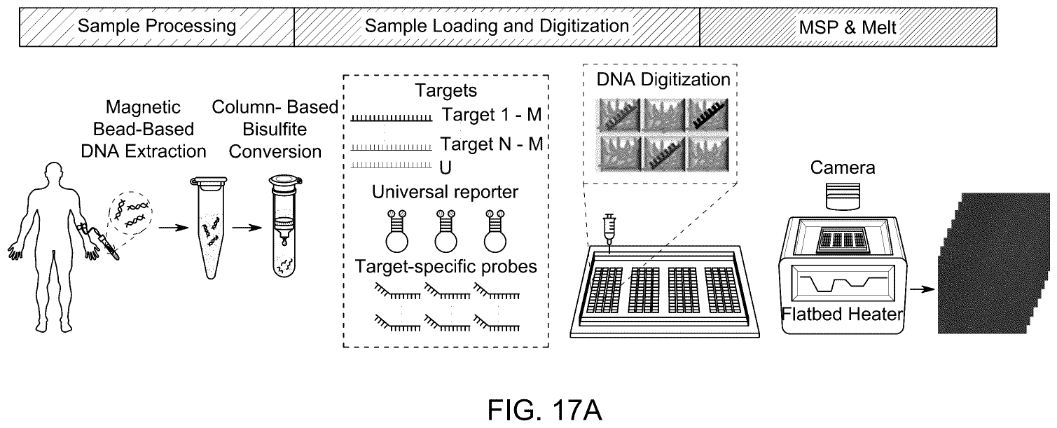

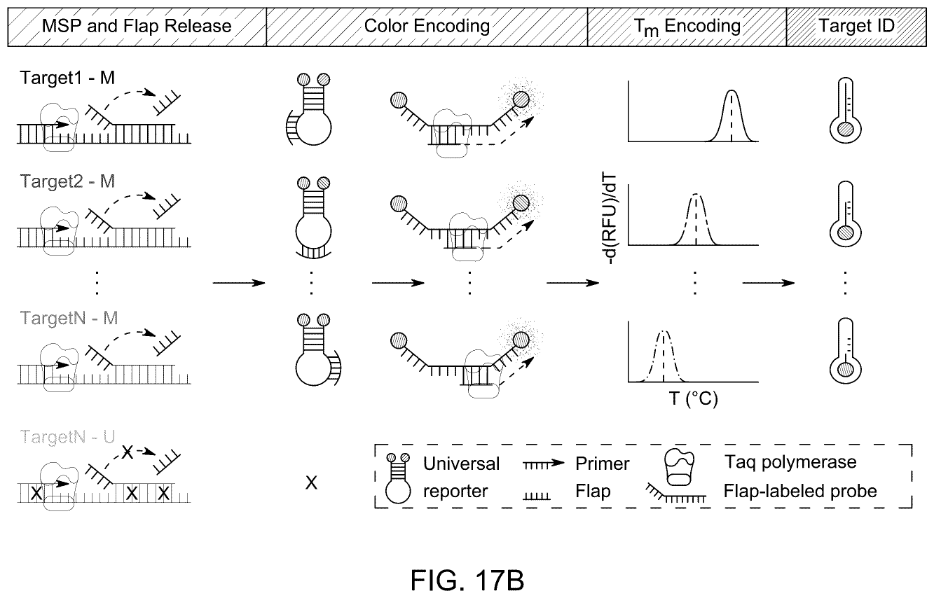

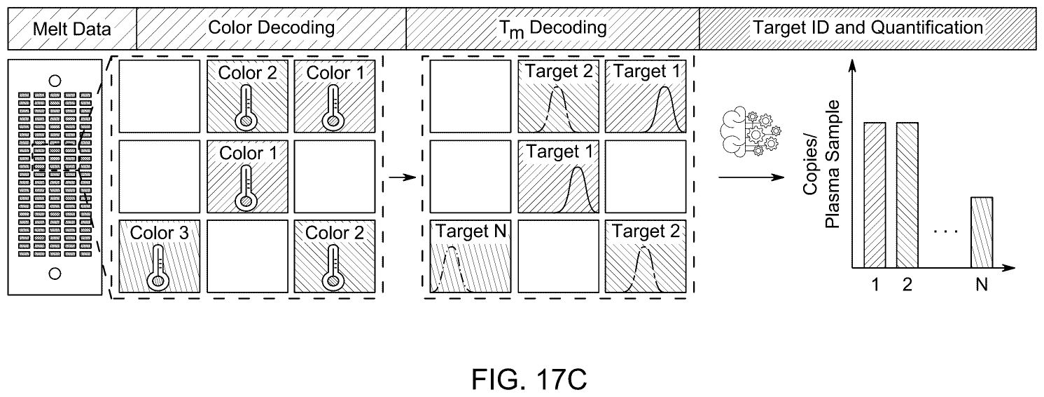

We developed a microfluidic platform that can simultaneously detect temperature-based and color-based signals. The overall assay workflow is illustrated in FIG. 17A. Starting from sample processing, cell-free DNA (cfDNA) is first extracted using silica-coated magnetic beads from 1 mL of plasma derived from peripheral blood drawn and subsequently undergoes column-based bisulfite conversion. During bisulfite conversion, unmethylated cytosines will be converted to uracils and get amplified in subsequent PCR reactions as thymines, whereas methylated cytosines will remain intact. The bisulfite converted cfDNA will then be added into the PCR reaction master mix. Upon mixing, the cfDNA-containing master mix will get loaded into the microfluidic device via vacuum and digitized via pressure, as described in our previous work. The digitized device will then be placed on a flatbed heater and undergoes methylation-specific PCR (MSP) for target amplification along with subsequent multicolor melt curve analysis for target identification. During melt curve analysis, a mirrorless interchangeable-lens camera (MILC), staged on top of the flatbed heater along with a set of four-color emission and excitation filters, collects melt images in different fluorescent channels for downstream data analysis.

There are several key design considerations of our assay that have enabled highly specific and highly multiplexed methylation detection (FIG. 17B). The assay begins with the hybridization of methylation-specific primers and 5′ flap-labelled methylation-specific probes to methylated targets, where the specificity is ensured by the incorporation of multiple CG loci in the sequence designs. During MSP, Taq polymerase with endonuclease activity extends the primer and cleave off 5′ flap sequence on the target-specific probe. After MSP, the cleaved flap hybridizes to and gets extended along its corresponding universal reporter, resulting in the separation of the fluorophore and quencher on the reporter and an increase in fluorescence. During the subsequent melt, the dissociation of the duplex results in a decrease in fluorescence. The change in fluorescence level over temperature increment is used to obtain a raw melt curve. The derivative of the melt curve is then calculated to find the temperature at the inflection point of the melt curve termed melting temperature Tₘ, the temperature at which exactly half of the duplex becomes denatured. Target identification can be achieved through combinations of Tₘ(s) and the fluorescent colors of the reporter. On the other hand, the methylation-specific primers and probes will have lower or no binding affinity to unmethylated targets due to CG-loci mismatches and therefore will not generate detectable signal in the post-MSP melt curve analysis.

Upon collecting a series of melt images of the microfluidic device, a customized MATLAB program is used for data analysis. First, a user-defined mask is applied to the aligned stack of melt images and the average fluorescent intensity values for the nanowells are extracted at each temperature point to obtain a mixture of melt peak curves representing all nanowells according to our previously published protocol. To assist efficient, accurate, and reliable target classification, three support vector machine classifiers are constructed, one for each fluorescent channel. Specifically, the extracted melt peak curves are fed into the corresponding classifier, and targets present in each fluorescent channel are classified and enumerated.

### Multiplexing Capability Validation

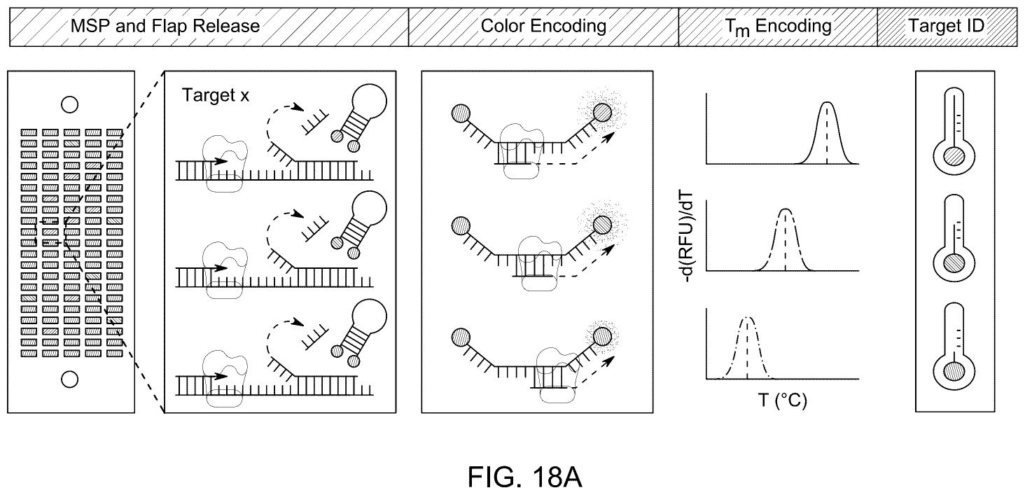

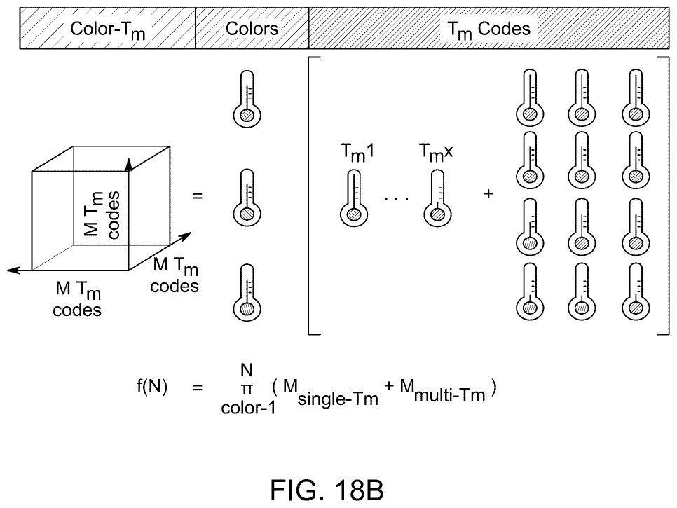

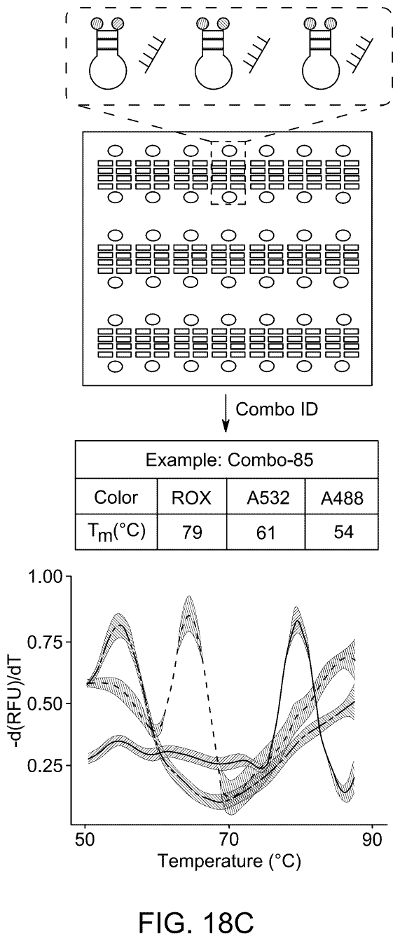

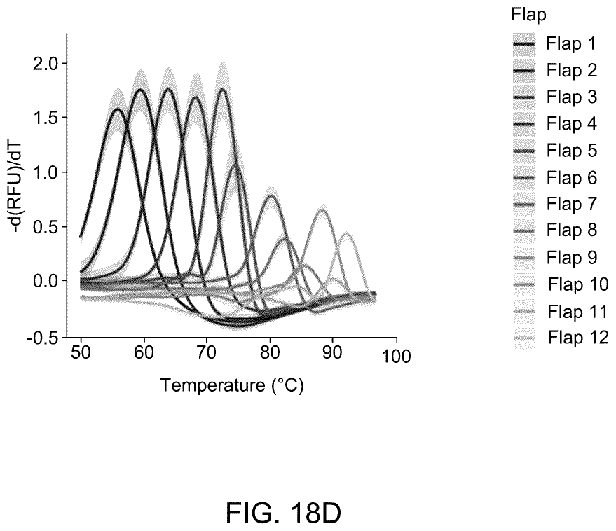

The digital nature of our PCR approach further enhances multiplexing capacity by enabling combinatorial encoding. Specifically, for a given target x, multiple flap-labeled probes can be designed, each containing a unique 5′ flap sequence while sharing an identical target-binding region (FIG. 18A). In a nanowell containing target x, these distinct flap-labeled probes hybridize to the target sequence during dMSP, leading to the cleavage of multiple flap sequences. The cleaved flaps subsequently hybridize to their respective universal reporters, generating melt curves with multiple Tₘ-color combinations.

To estimate the theoretical multiplexing potential of this encoding strategy, we applied a general formula to calculate the number of possible combinatorial target identification signatures as follows

$$f ( N ) = {\prod }_{1}^{N} ( M + 1 ) - 1$$

where N represents the number of color channels and M denotes the number of available Tₘ codes per channel (FIG. 18).

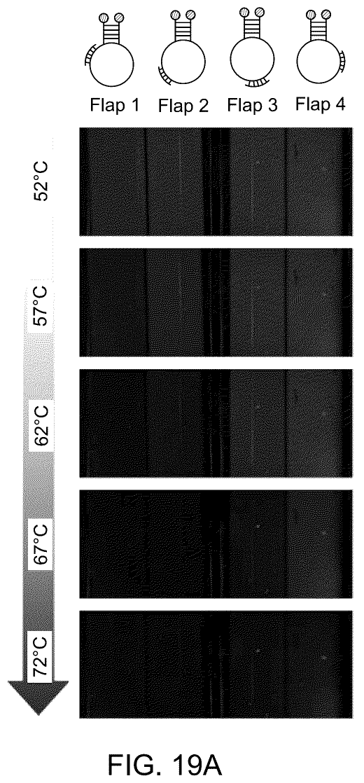

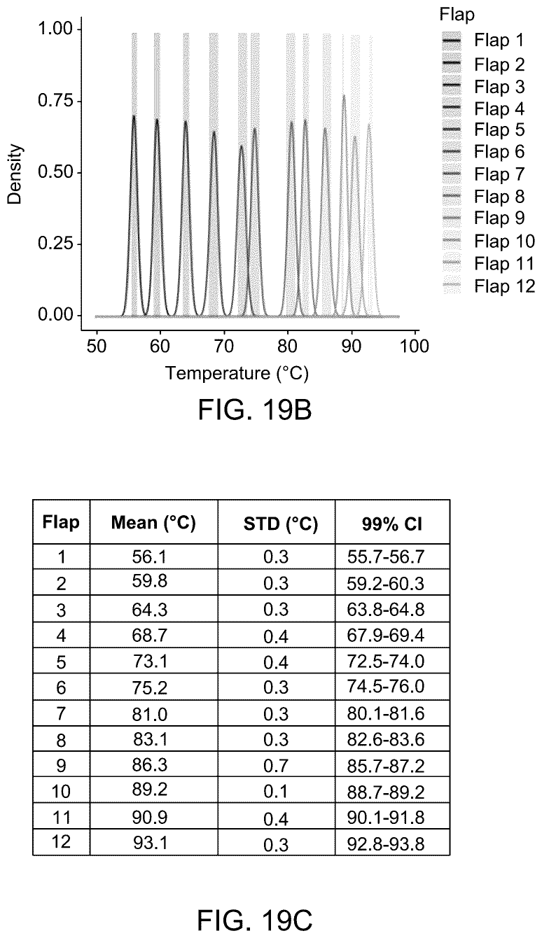

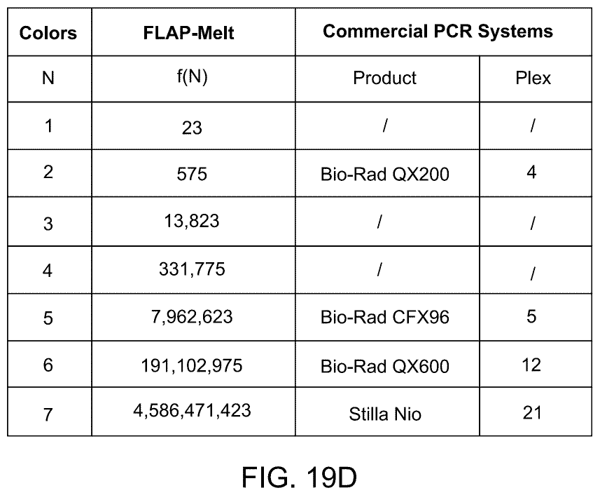

To determine the number of available Tₘ codes in our three-color melt setup, we first assessed the Tₘ resolution within a single fluorescence channel, specifically evaluating the maximum number of distinct Tₘ codes detectable in a single color. We selected the ROX channel as a model and prepared a reaction master mix containing universal reporters labeled with ROX. Two ROX-labeled universal reporters were used to cover a temperature range of 50 to 100° C. Synthetic flap sequences, each complementary to a distinct location on the two universal reporters, were spiked into separate reactions and loaded into individual modules on our microfluidic device. We successfully detected 12 distinct melt peak populations, corresponding to 12 unique flap sequences with well-separated Tₘ(s) (FIG. 18D). A probability density function analysis was employed to determine the melting temperature resolution. A minimum temperature gap of 2.5° C. between any two targets is required for 99% confident discrimination (FIG. 19). Further statistical analysis confirmed the reproducibility and robustness of our multicolor melt platform, as indicated by the consistency between the means and medians of Tₘ distributions, with standard deviations ranging from 0.1 to 0.7° C. (FIG. 19). Notably, our setup far exceeds the multiplexing level of commercially available digital PCR platforms, such as the Bio-Rad QX600 and Stilla Nio, achieving orders of magnitude higher target detection capacity.

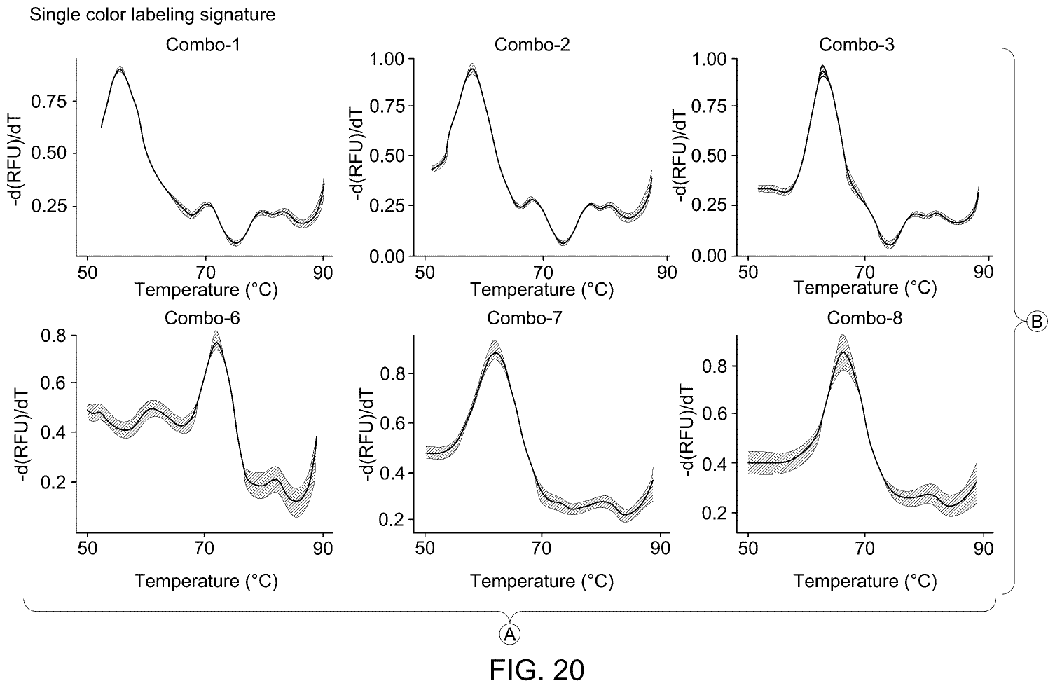

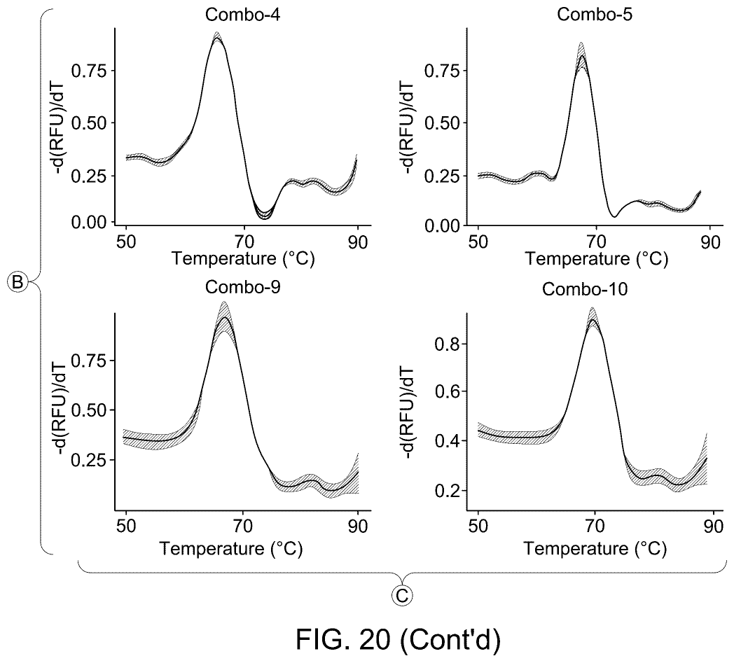

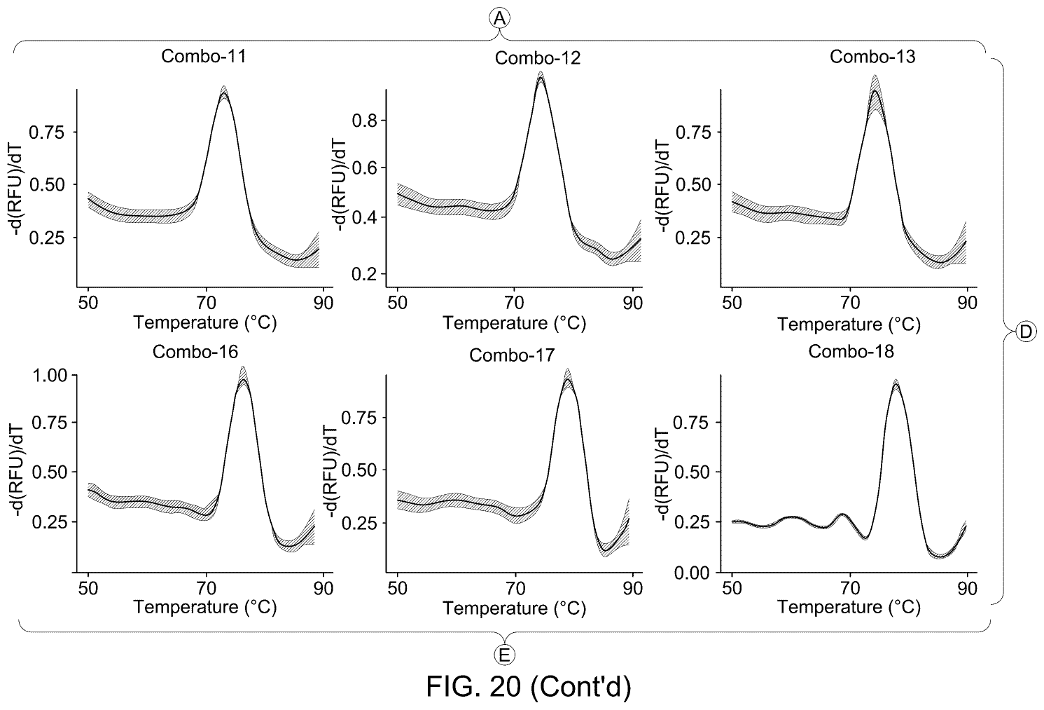

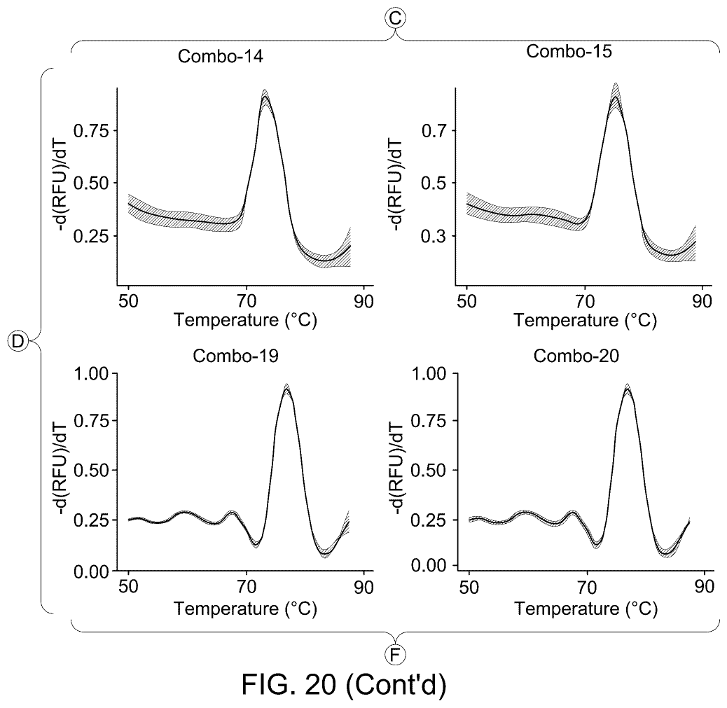

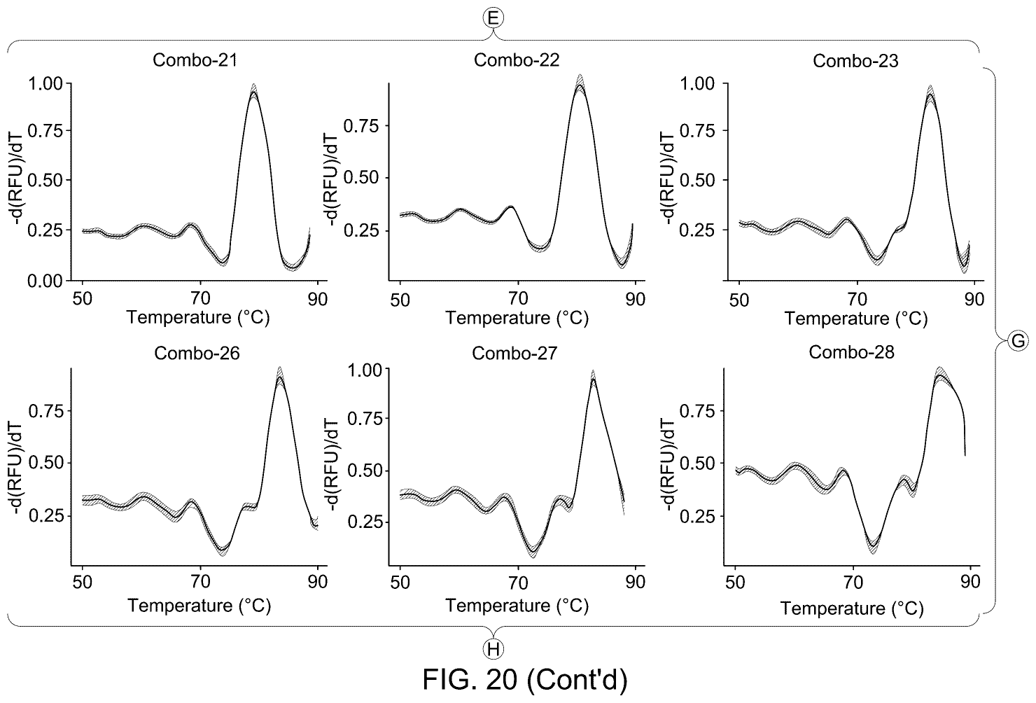

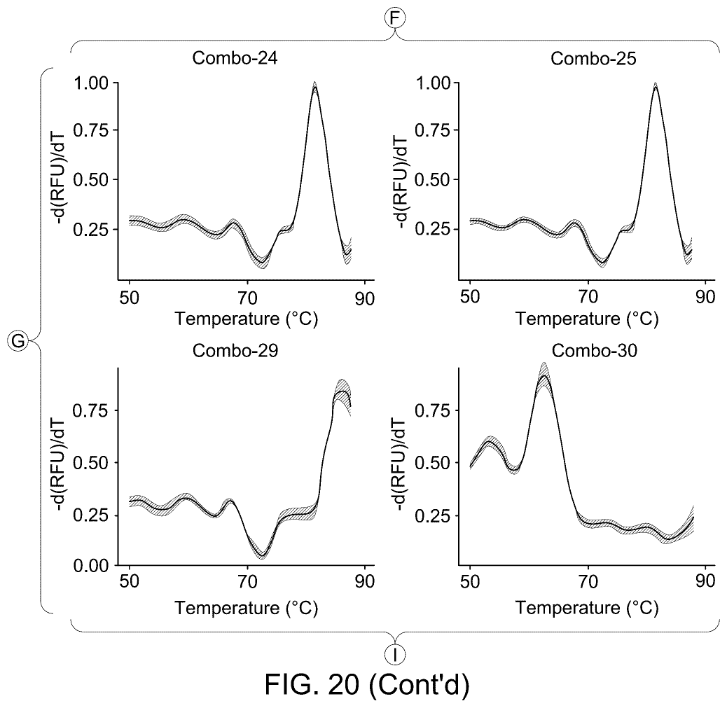

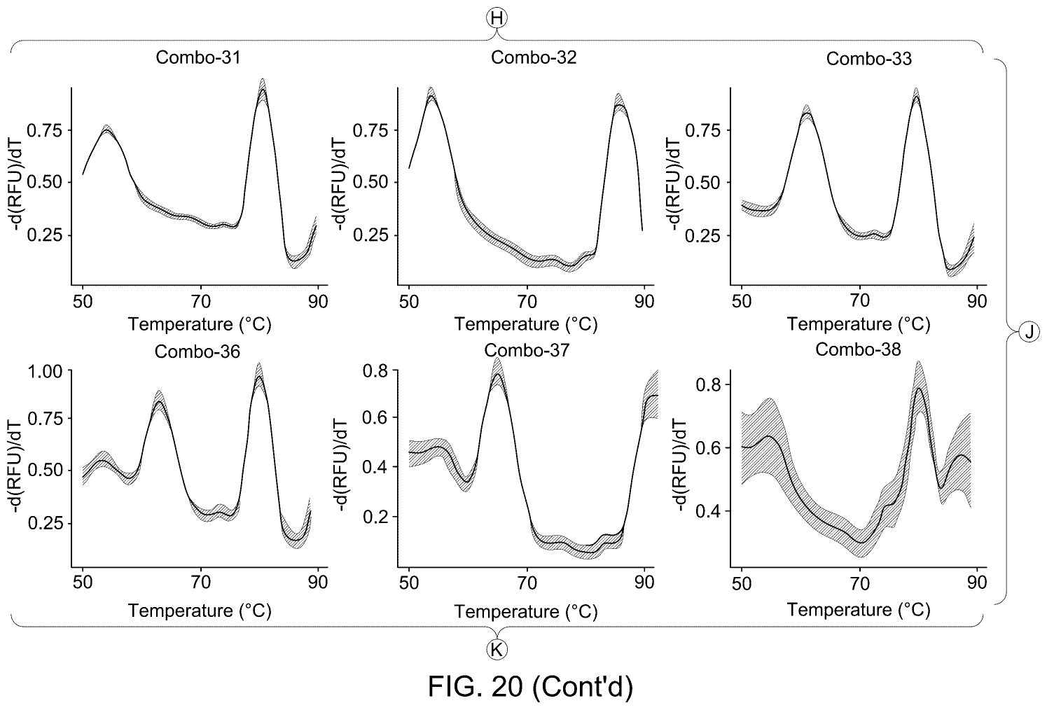

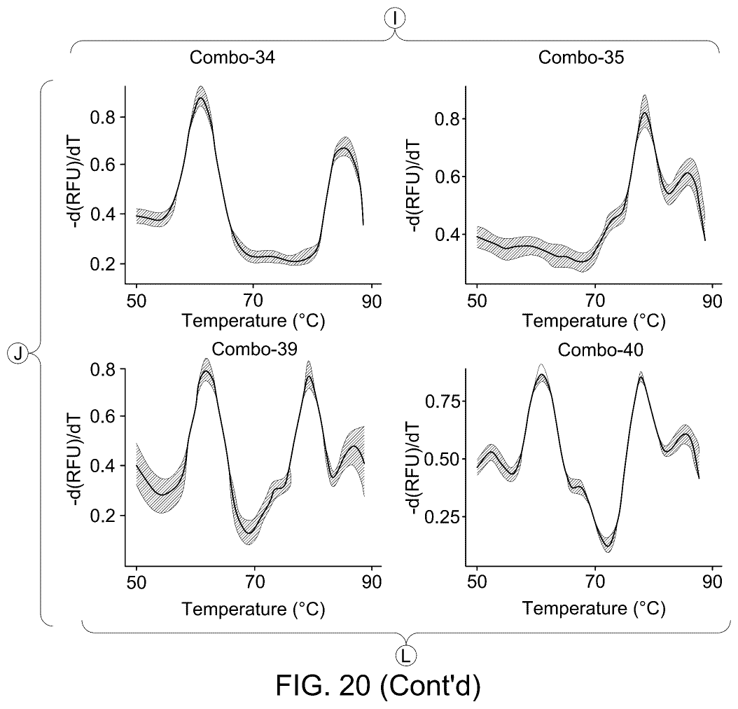

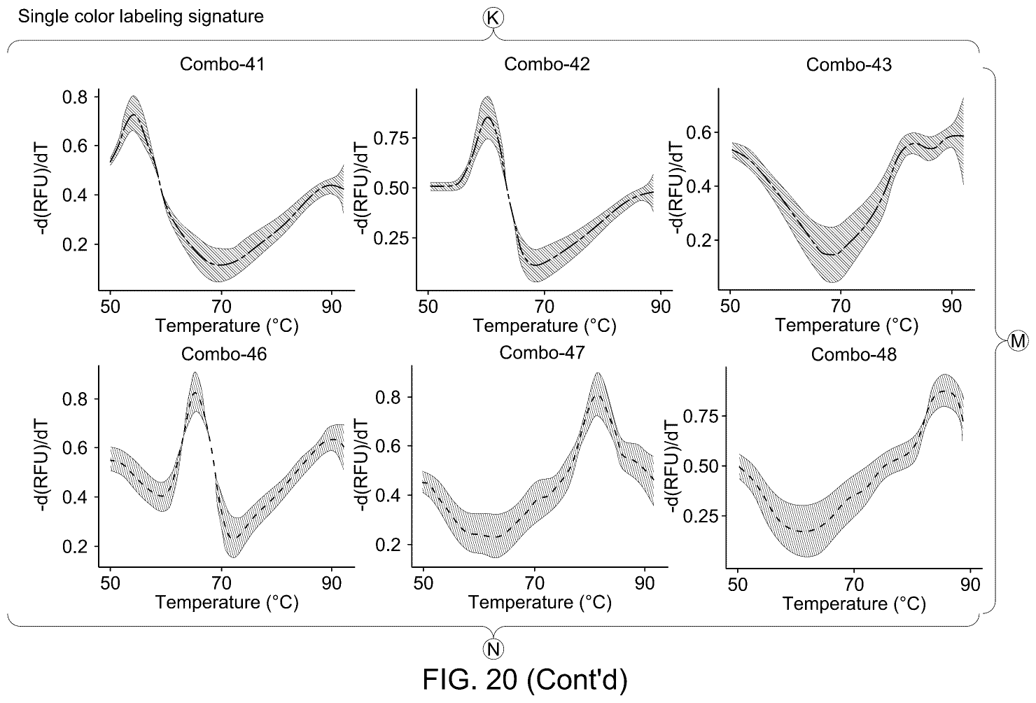

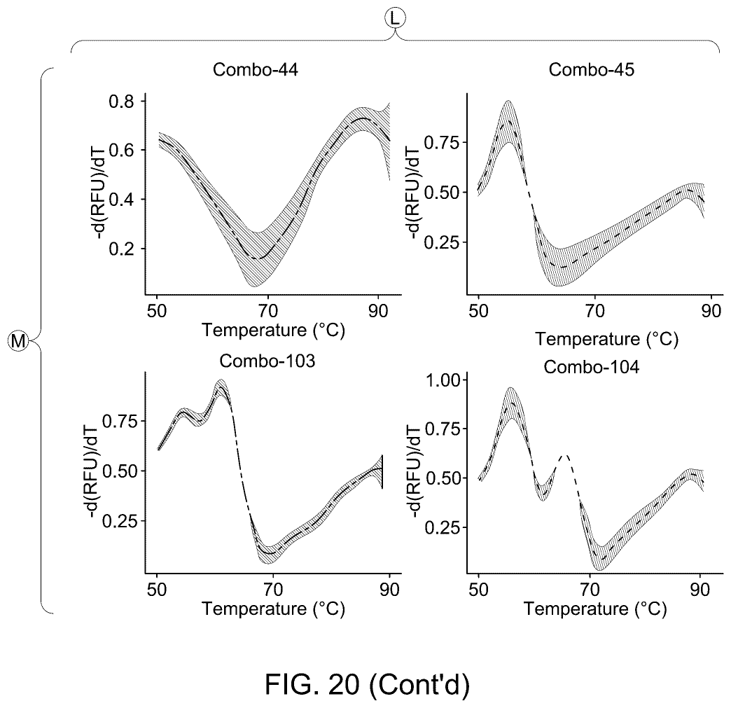

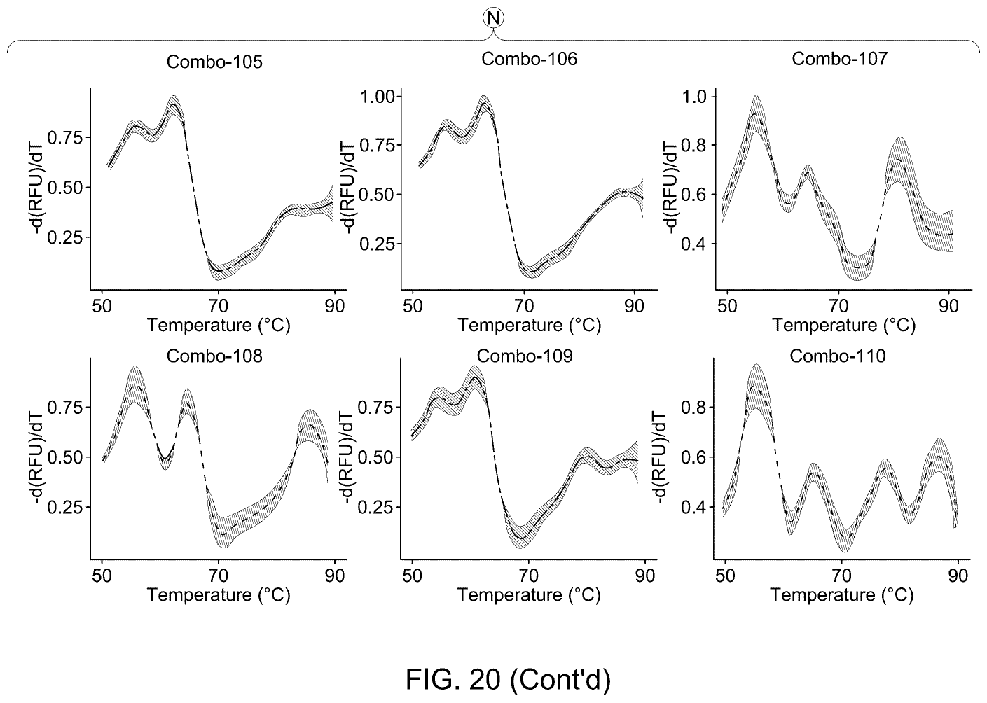

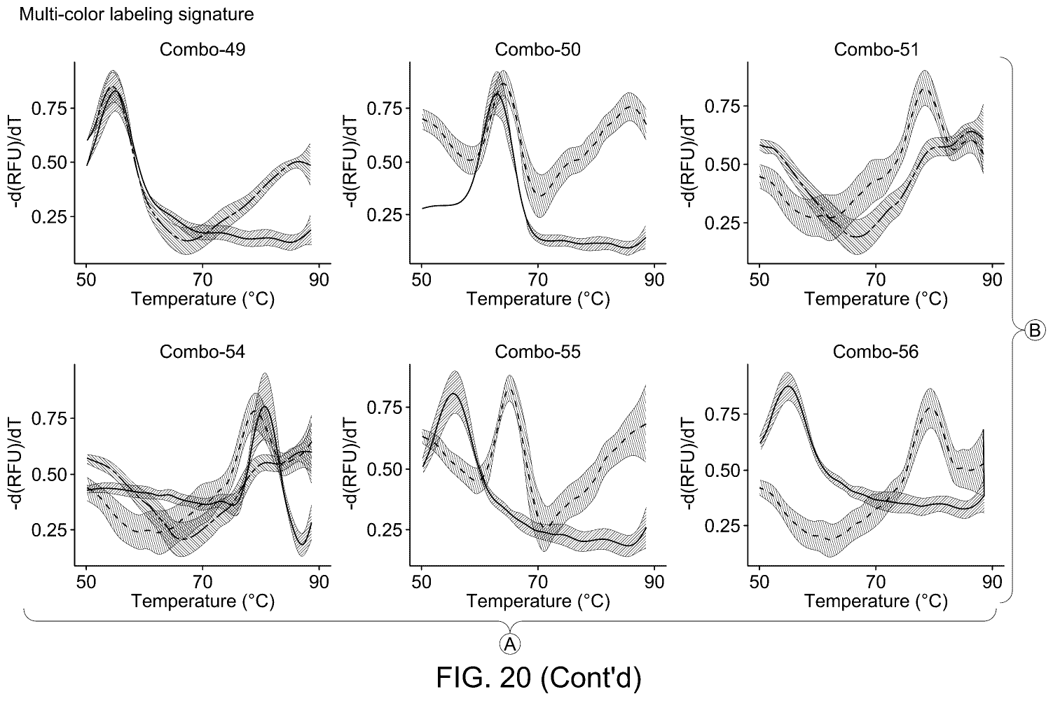

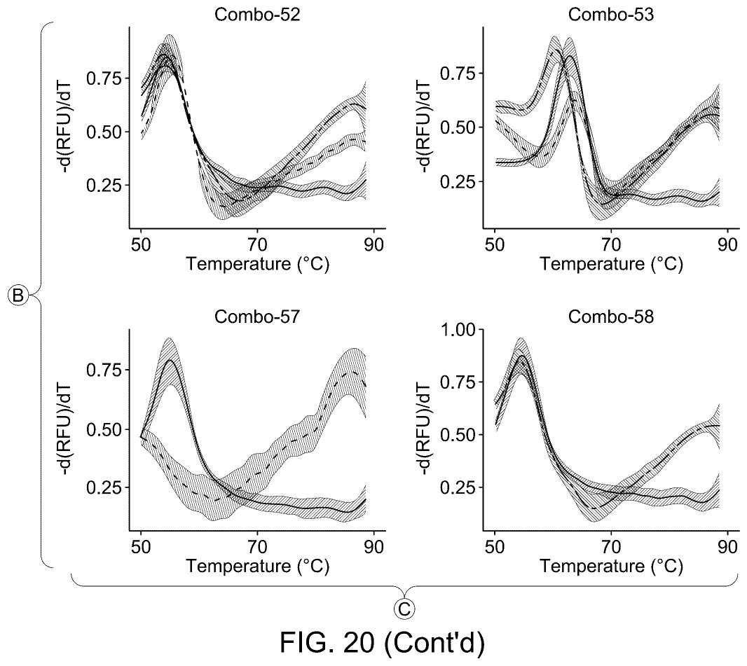

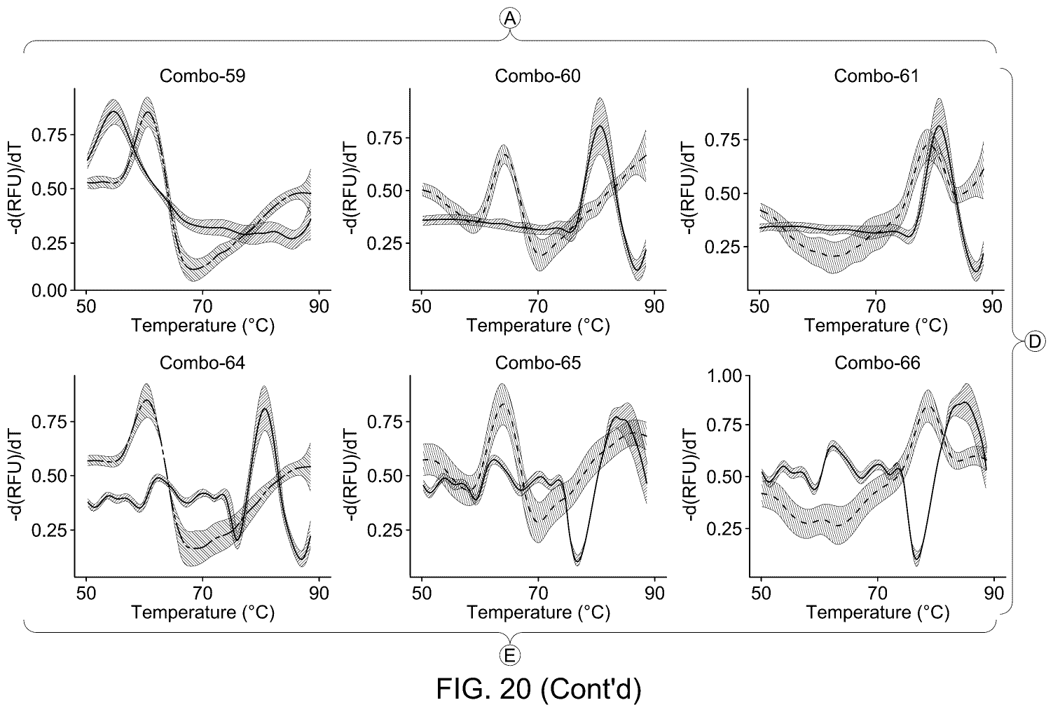

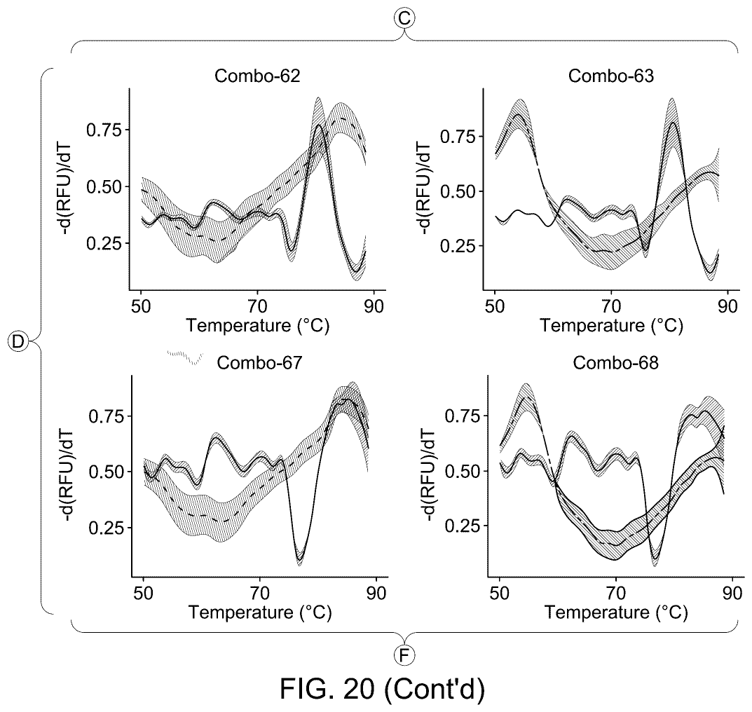

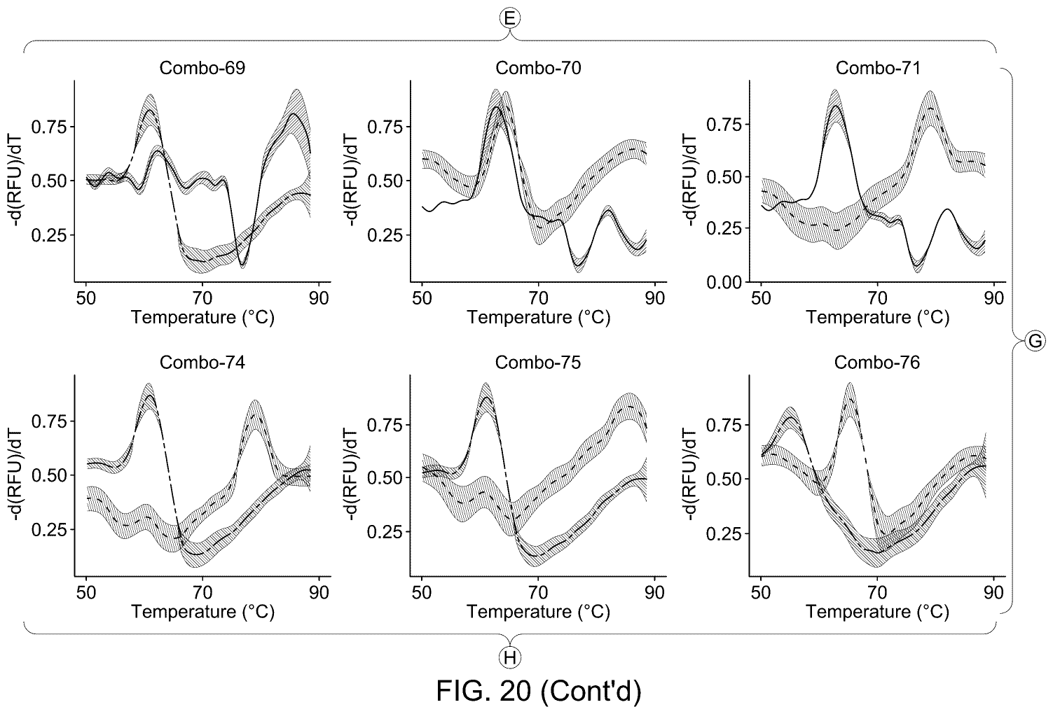

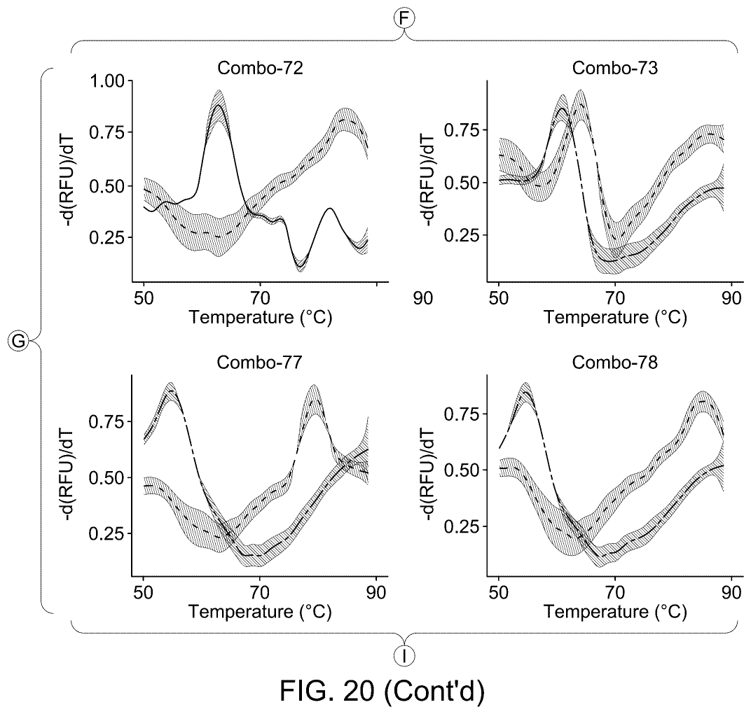

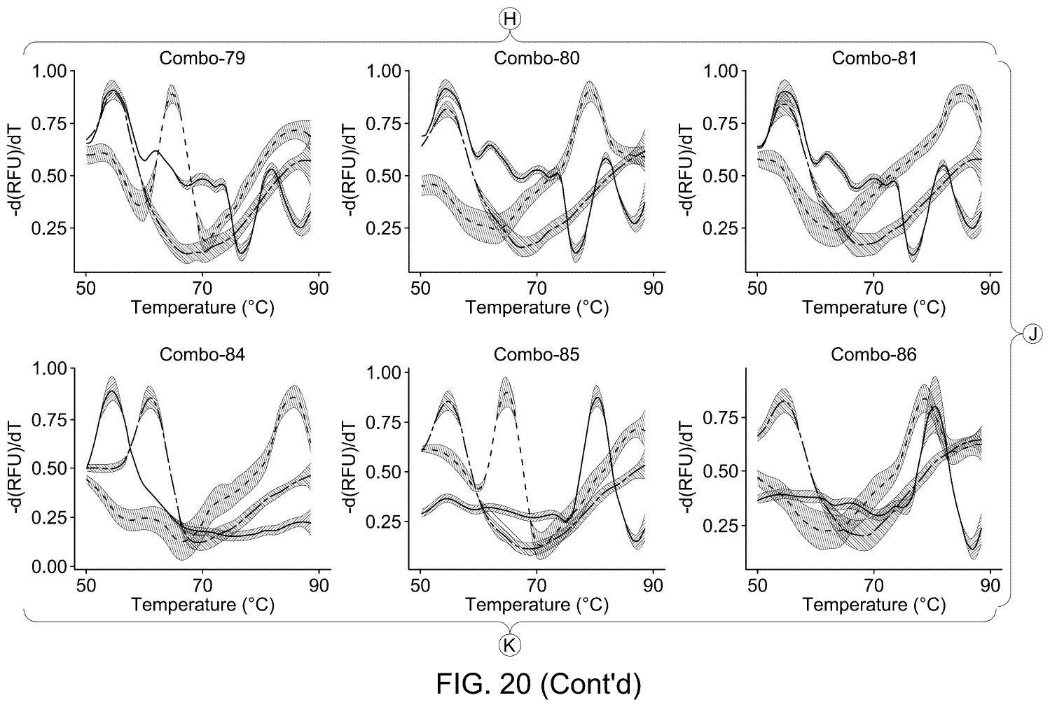

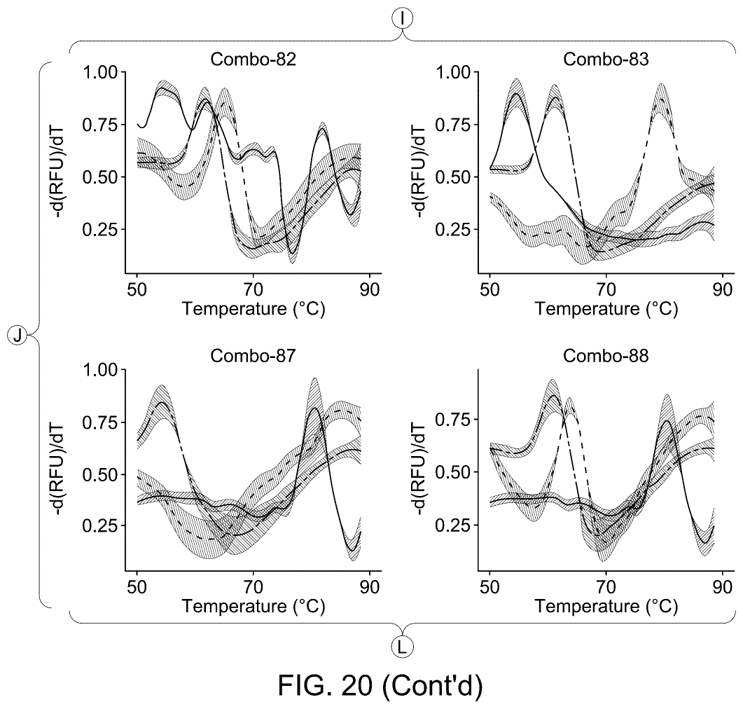

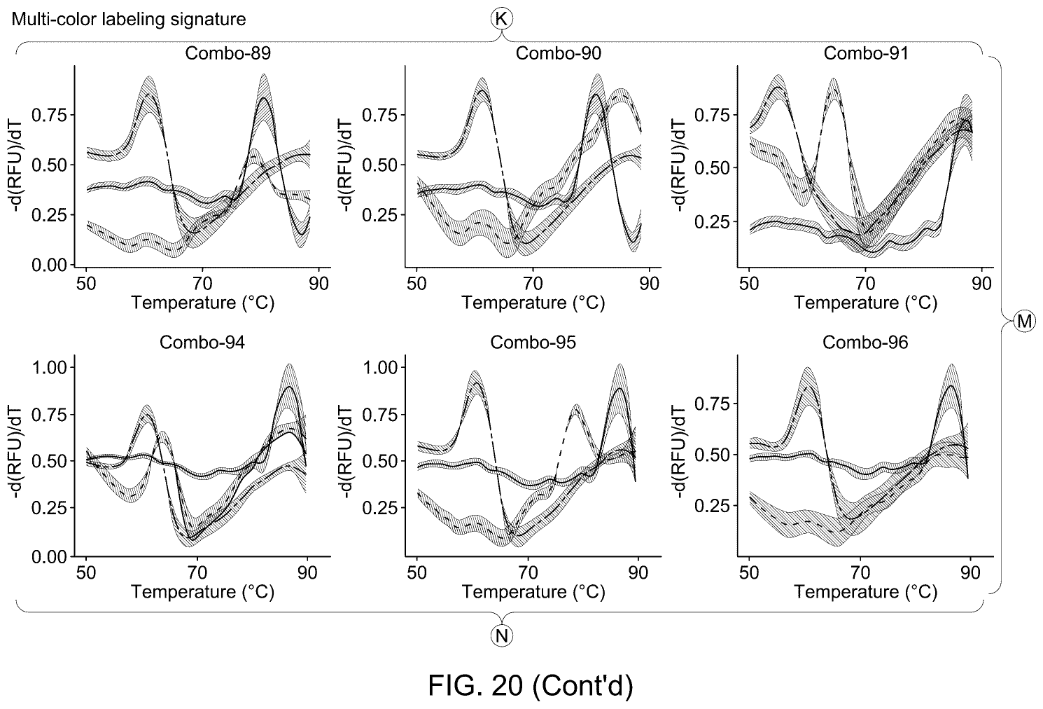

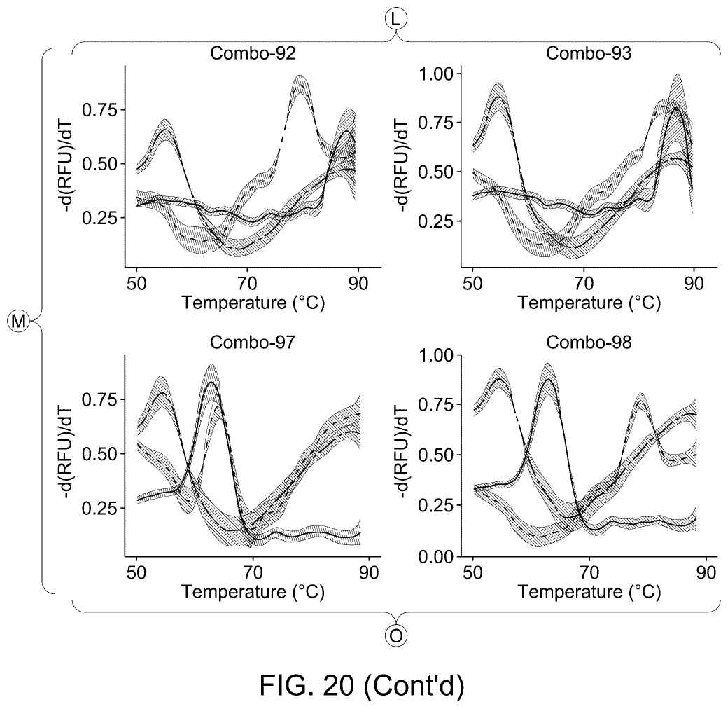

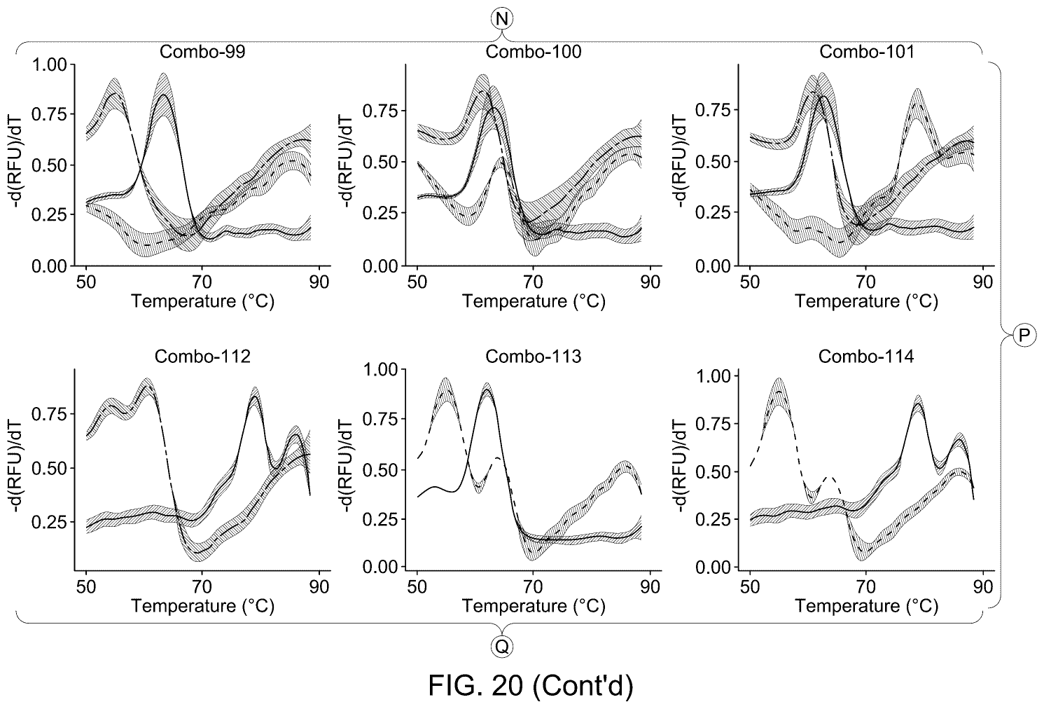

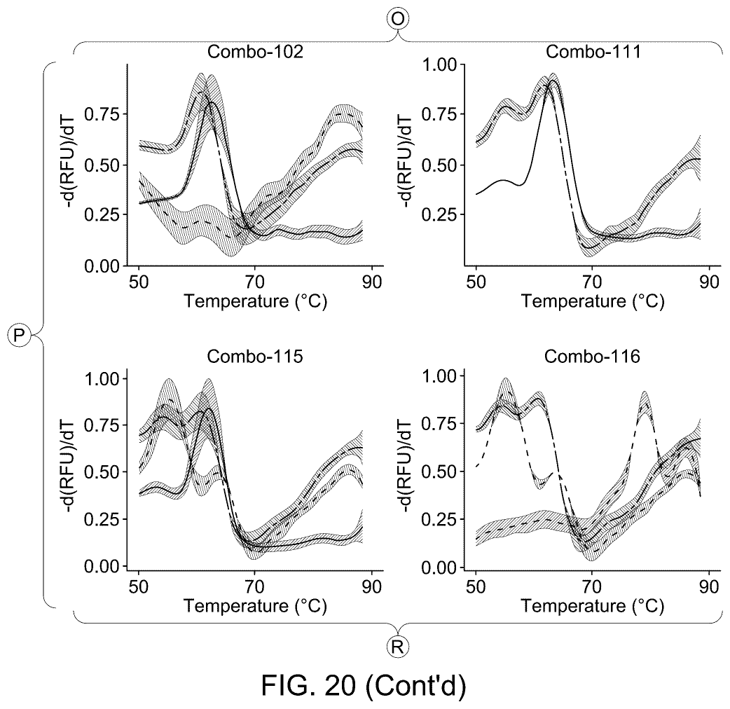

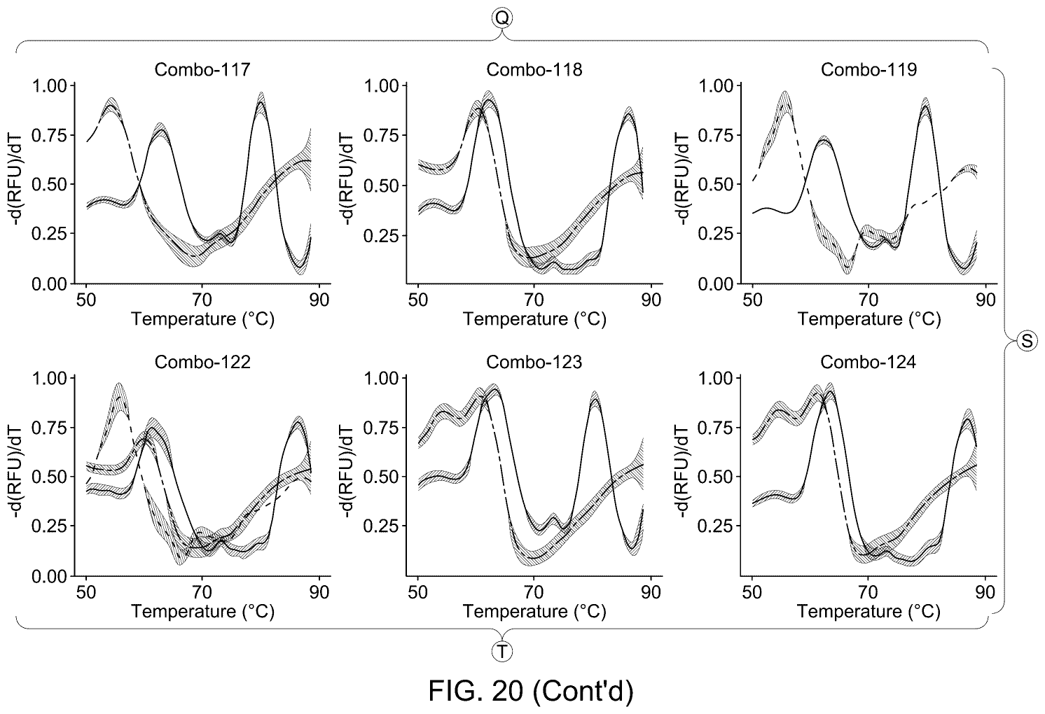

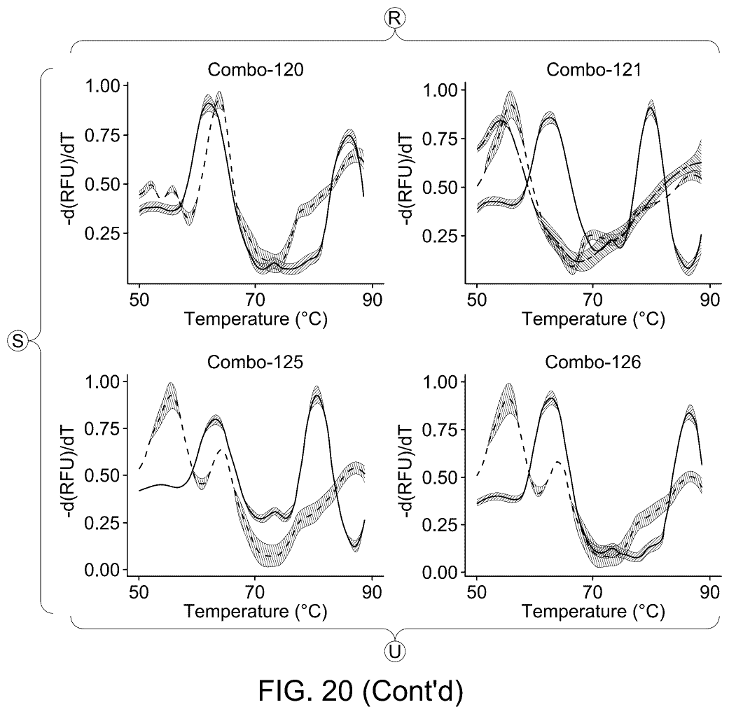

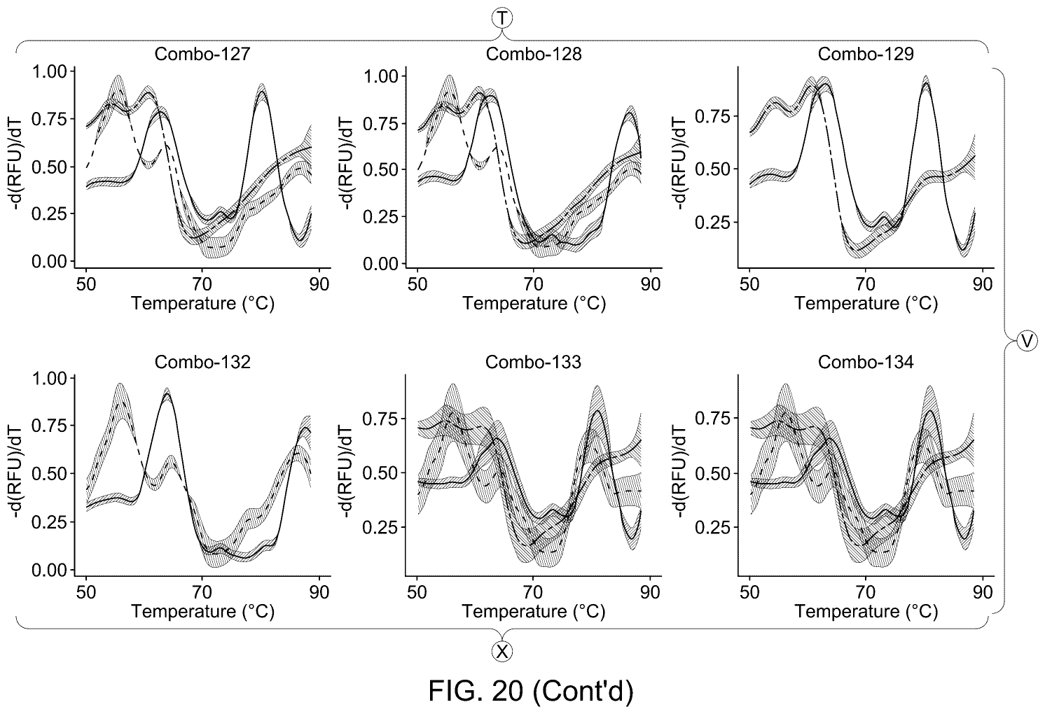

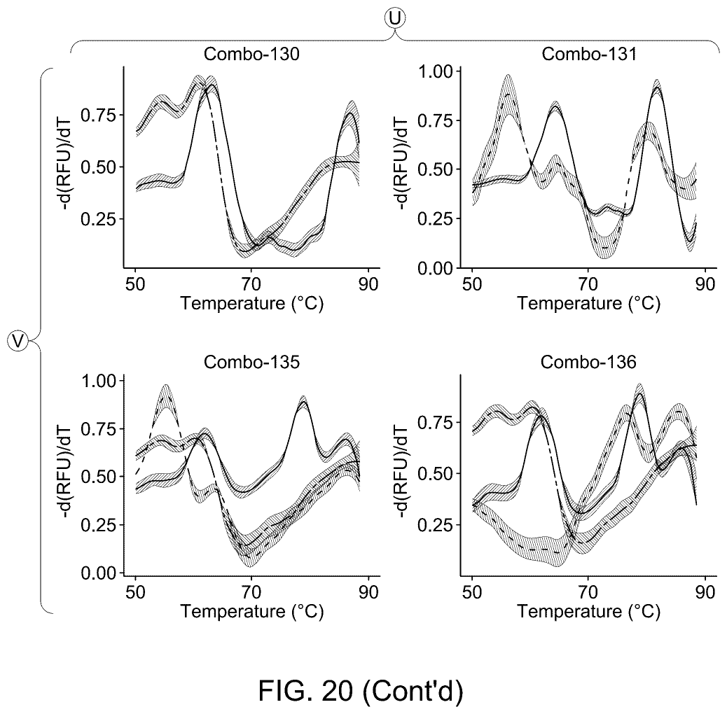

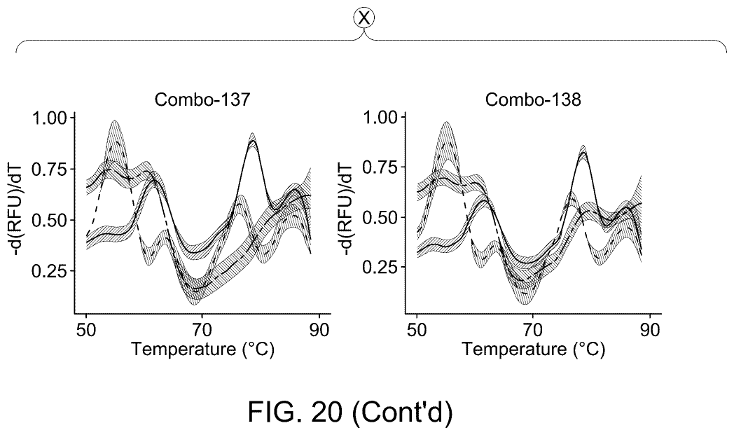

To experimentally validate the feasibility of combining labeling colors and melting temperatures for high level multiplexing labeling, we performed a flap-hybridization-extension assay by spiking in combinations of synthetic flaps alongside universal reporters at concentrations mimicking post-MSP products. These reaction mixtures were loaded into distinct modules on the microfluidic chip (FIGS. 18C and 19). For example, a target encoded by Combo-85 as its unique signature was represented by melting derivative peaks detected at 79° C. in the ROX channel, 62° C. in the Alexa 532 channel, and 54° C. in the Alexa 488 channel (FIG. 18C). We successfully demonstrated the detection of 138 unique labeling signatures, encompassing a range of composition scenarios, including single-Tₘ-single-color, single-Tₘ-multiple-color, multiple-Tₘ-single-color, and multiple-Tₘ-multiple-color combinations (FIG. 20). Across all combinations, we consistently observed melt curves with expected Tₘ(s). Notably, even in reaction mixtures containing up to four Tₘ codes per channel, resulting in a total of 12 Tₘ codes across the three-color system, the expected melt peaks remained well-defined, further demonstrating the robustness and scalability of our multiplex encoding strategy (FIG. 19D).

### Multiplex Detection of Methylation Biomarkers by FLAP-Melt

For proof-of-concept demonstration, we focused on non-small cell lung cancer (NSCLC) detection using a panel of 15 methylation biomarkers, including a previously validated 4-gene panel targeting NSCLC and additional 11 genes discovered for universal cancer detection. We reasoned that appending a panel of universal cancer detection biomarkers with validated specificity into a cancer-specific biomarker panel should improve the detection probability of ultra-low copy level of DNA targets, which in turn can enhance the clinical sensitivity of the assay. According to our in-silico simulation, expanding the 4-gene NSCLC panel into the 15-gene panel to achieve a sensitivity of 98% and specificity of 100%.

Developing a highly multiplexed PCR reaction presents several key challenges, including nonspecific interactions (e.g., primer-dimer formation), amplification competition among targets of varying concentrations, and limited capacity for target identification using a restricted number of fluorescent channels. The FLAP-Melt assay addresses these challenges through a combination of innovative strategies. By employing dual Tₘ-color encoding, FLAP-Melt significantly expands the number of target labeling signatures using limited fluorescent channels, while digitization on a microarray platform effectively minimizes amplification competition by spatially isolating individual reactions. To further mitigate nonspecific interactions, such as primer-dimer formation, we incorporated the Homo-Tag Assisted Non-Dimer System (HANDS), which adds a universal 5′ tail to all primers. During early PCR cycles, target amplification is driven by specific primer sequences, while later cycles are dominated by the universal tails. In the event of primer-dimer formation, the universal tails self-anneal into pan-handle structures, halting further amplification. This mechanism enhances reaction specificity and conserves reagents, ensuring robust performance even in highly multiplexed reactions. Collectively, these strategies enable FLAP-Melt to overcome the typical hurdles of multiplexed PCR, achieving precise, highly parallelized target detection with minimal nonspecific amplification.

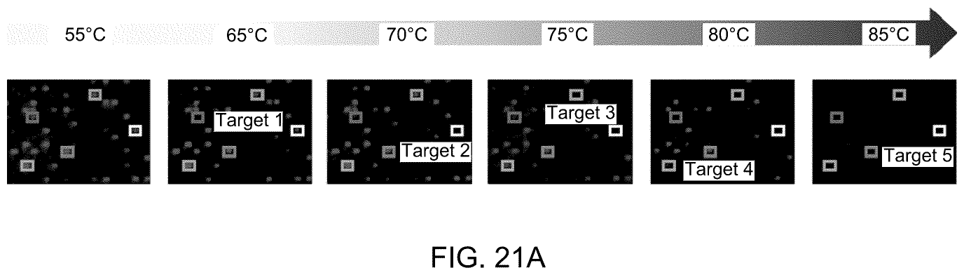

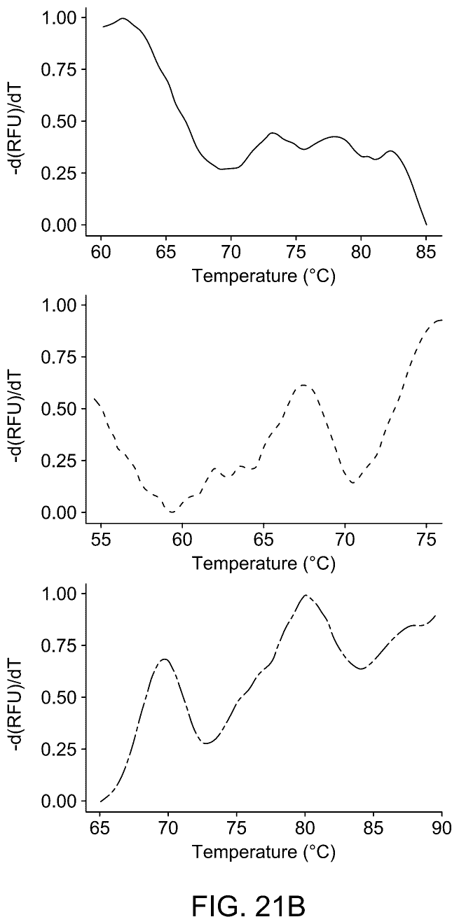

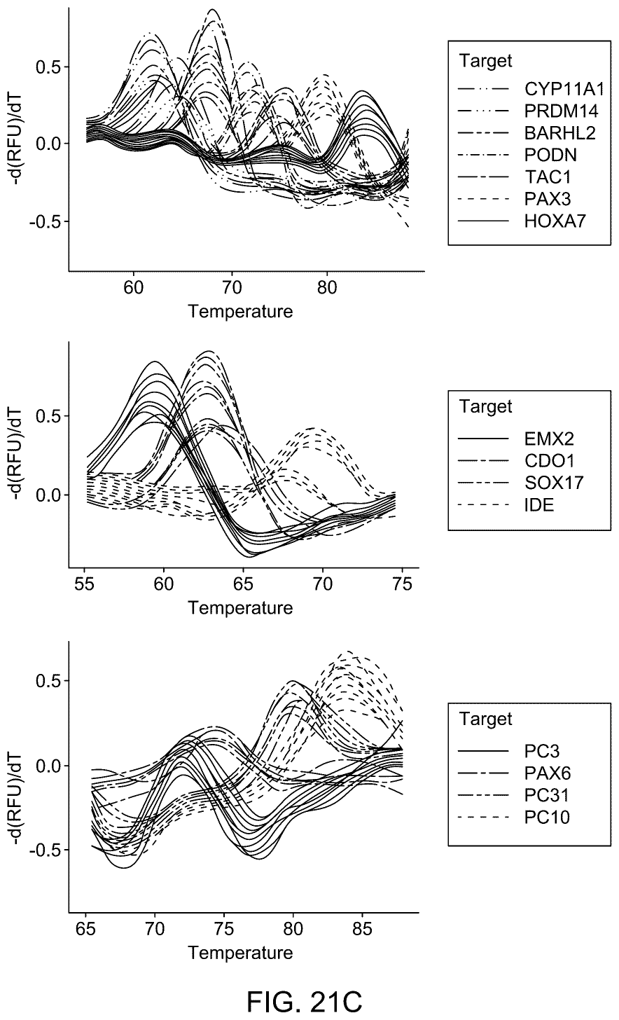

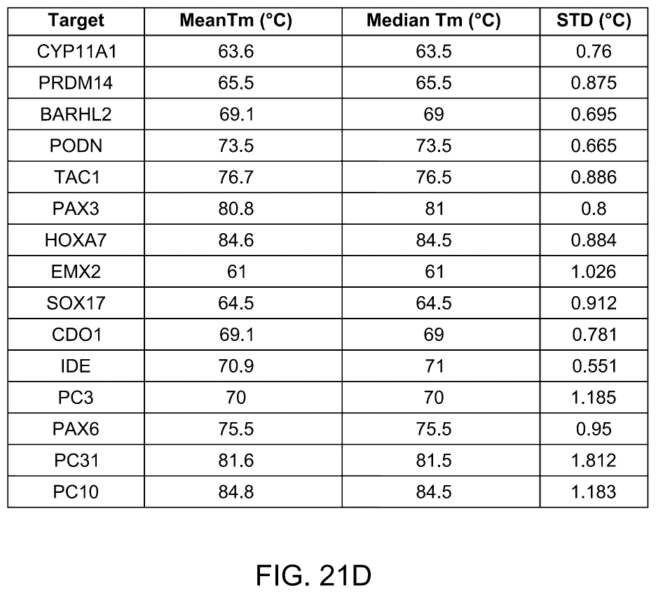

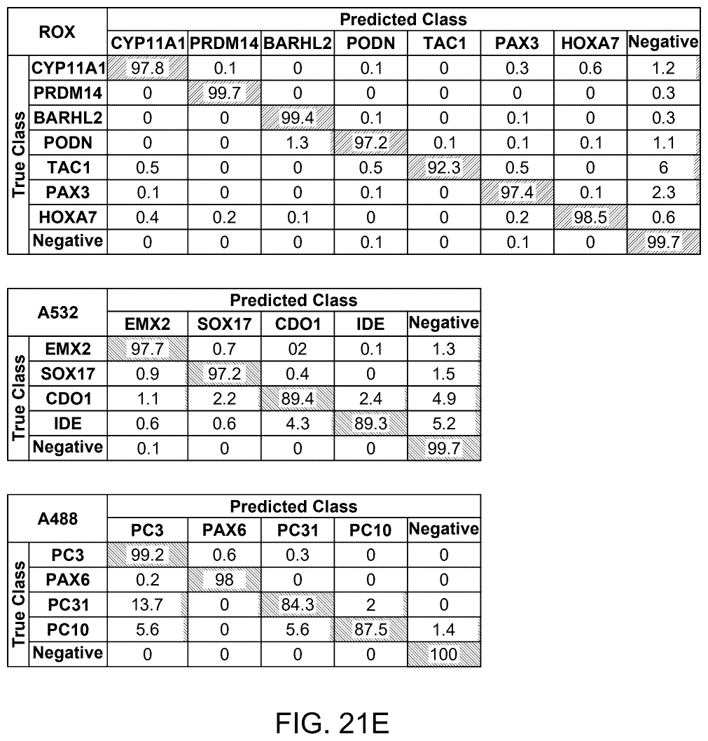

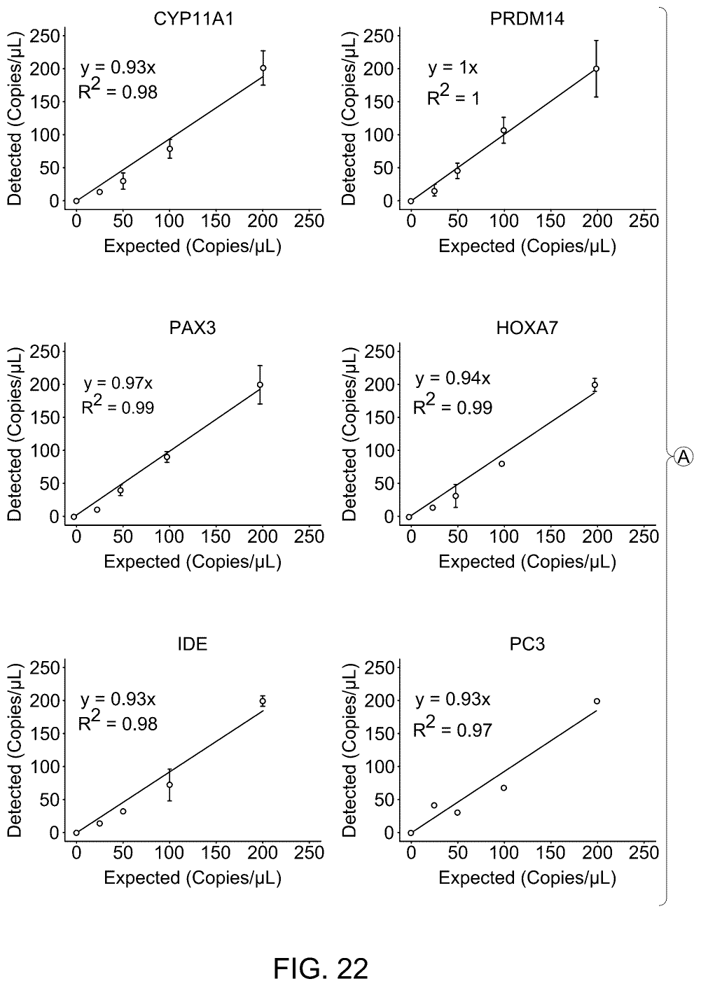

To detect the 15 biomarkers simultaneously, we utilized three fluorescent channels: ROX, Alexa Fluor 532, and Alexa Fluor 488 (FIG. 21). These channels were optimized to detect 7, 4, and 4 biomarkers, respectively, following the design guidelines established above. Series of melt images of the entire chip during temperature ramping were collected for each channel separately. FLAP-Melt successfully detected all 15 biomarkers in a single reaction and generated distinct melt peak populations for each biomarker across all channels, enabling accurate enumeration of positive curves for target quantification (FIG. 21C). We further compared our digital MSP assay to the gold-standard bulk MSP assay. Under identical assay conditions, the bulk assay produced single composite melt peak curves in each channel, where the curves failed to clearly represent all expected targets (FIG. 21B). This could be attributed to the competition between targets that has led to biased amplification efficiency and the limited temperature resolution of the bulk assay instrument, which hindered the distinction of closely spaced melt peaks. A statistical analysis of the digital Tₘ values for all 15 targets, including a tight distribution of standard deviations, further highlights the assay's reproducibility and precision (FIG. 21D). The mean and median Tₘ values confirm a minimal 2° C. difference between targets, in line with the design rule established in FIG. 19. The distinct Tₘ values ensure reliable target differentiation in the highly multiplexed setup. Variability in targets like PC31 and PC10 likely arises from thermal and optical noise on the Alexa Fluor 488 channel, which could be minimized through further optimization. During assay development, efforts were made to space out Tₘ values to ensure reliable target distinction despite these variations.

### Analytical Validation of FLAP-Melt

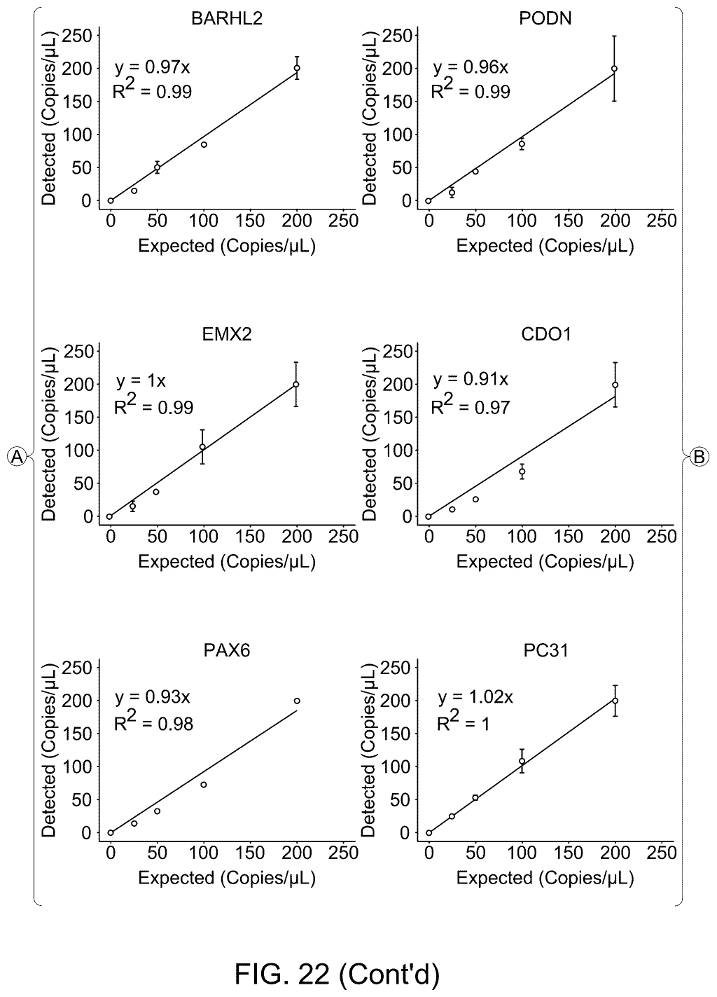

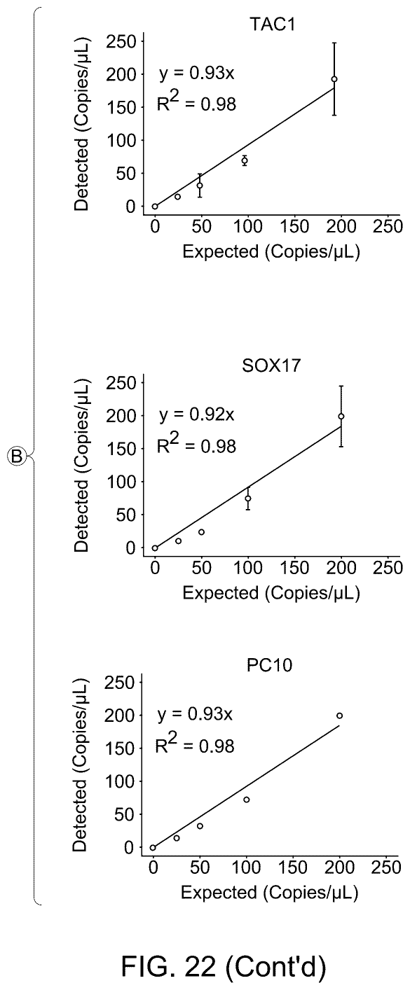

To assess the analytical performance of FLAP-Melt, we first used synthetic equivalents of the bisulfite-converted methylated sequences of the 15 targets. Due to limited chip capacity, we randomly divided the 15 targets into three groups of five targets to maintain a reasonable dynamic range. For each target, methylated synthetic epialleles were serially diluted to concentrations ranging from 0 to 200 copies in the presence of 18 ng genomic unmethylated DNA, a concentration equivalent to genomic DNA background in a typical 1 to 2 mL plasma sample. Linear fits of the standard curves exhibited R² values >0.95 across all targets, indicating that the FLAP-Melt assay provides strong absolute quantification capabilities among a wide range of targets (Table 1 and FIG. 22). The analytical specificity of FLAP-melt for detection of multiple methylated targets was validated by ensuring that no cross-reactivity occurred between any methylated target and the unmethylated genomic DNA. Additionally, the linear fits of the standard curves yielded slopes >0.89 across all targets, suggesting that overall FLAP-Melt could detect at least 89% of the expected DNA which is comparable to our previously reported detection efficiency.

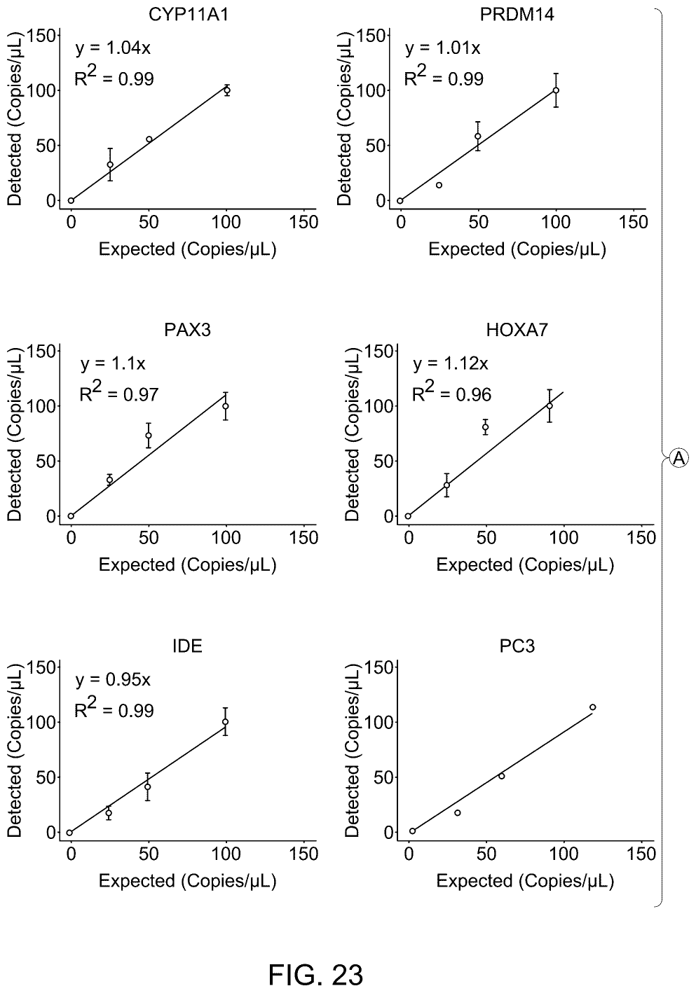

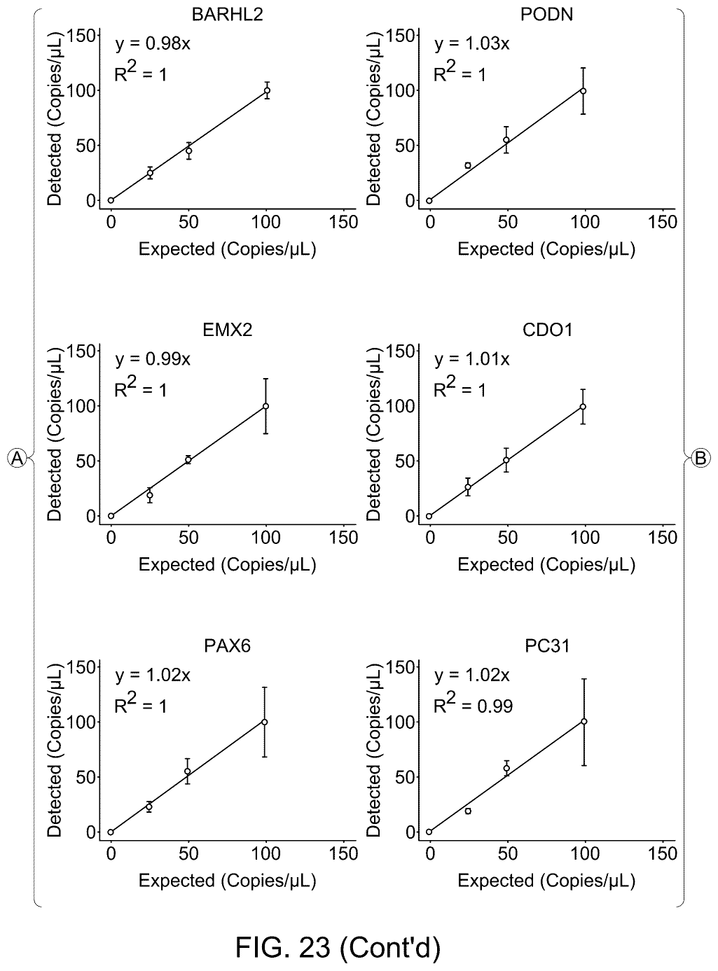

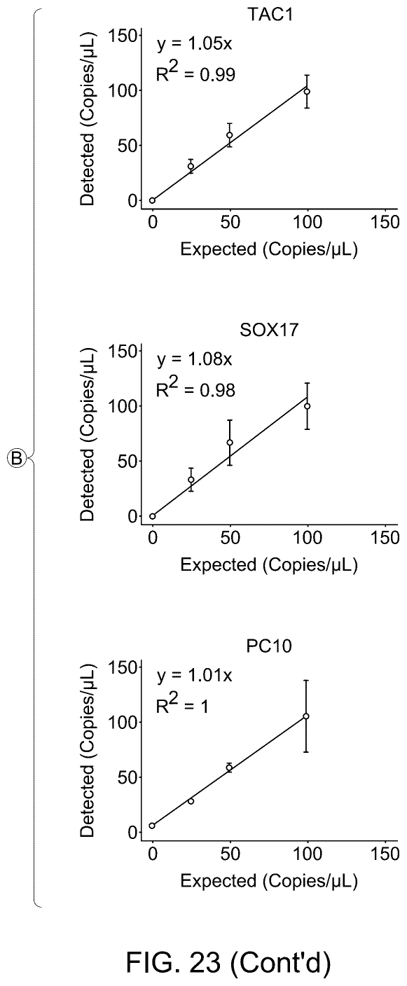

As a transition into more biologically complex samples, we further performed serial dilution of bisulfite-converted methylated genomic DNA at concentrations ranging from 0 to 100 copies per module in the presence of 18 ng unmethylated genomic DNA background. Similarly, linear fits of the standard curves exhibited R² values >0.96 across all targets (Table 1 and FIG. 23). The slopes of the linear fits range from 0.99 to 1.1. The slight discrepancies observed between the expected and detected copy numbers are likely due to minor inaccuracies in determining the stock concentration using the LINE-1 quantification assay since we observed an overall trend of higher detected copy number of target DNA than expected across most of the targets.

TABLE 1
Analytical validation of FLAP-Melt

| | Synthetic Methylated + | | Genomic Methylated + | |
|---|---|---|---|---|
| | Genomic Unmethylated | | Genomic Unmethylated | |
| Target | Slope | R² | Slope | R² |
| CYP11A1 | 0.94 | 0.98 | 1.04 | 0.99 |
| PRDM14 | 1.00 | 1.00 | 1.01 | 0.99 |
| BARHL2 | 0.98 | 1.00 | 0.98 | 1.00 |
| PODN | 0.96 | 0.99 | 1.03 | 1.00 |
| TAC1 | 0.93 | 0.98 | 1.05 | 0.99 |
| PAX3 | 0.97 | 0.99 | 1.10 | 0.97 |
| HOXA7 | 0.94 | 0.99 | 1.12 | 0.96 |
| EMX2 | 1.00 | 0.99 | 0.99 | 1.00 |
| CDO1 | 0.91 | 0.97 | 1.01 | 1.00 |
| SOX17 | 0.92 | 0.98 | 1.08 | 0.98 |
| IDE | 0.93 | 0.98 | 1.08 | 0.98 |
| PC3 | 0.93 | 0.97 | 1.00 | 1.00 |
| PAX6 | 0.93 | 0.97 | 1.02 | 1.00 |
| PC31 | 1.02 | 1.00 | 1.02 | 0.99 |
| PC10 | 0.89 | 0.95 | 1.01 | 1.00 |

### Biological Validation of FLAP-Melt

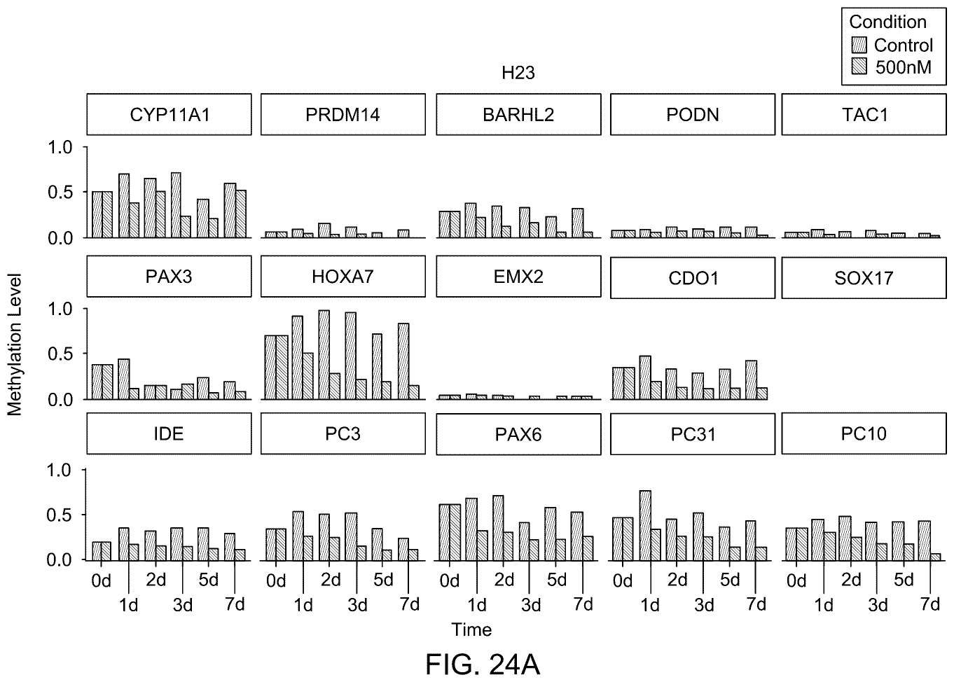

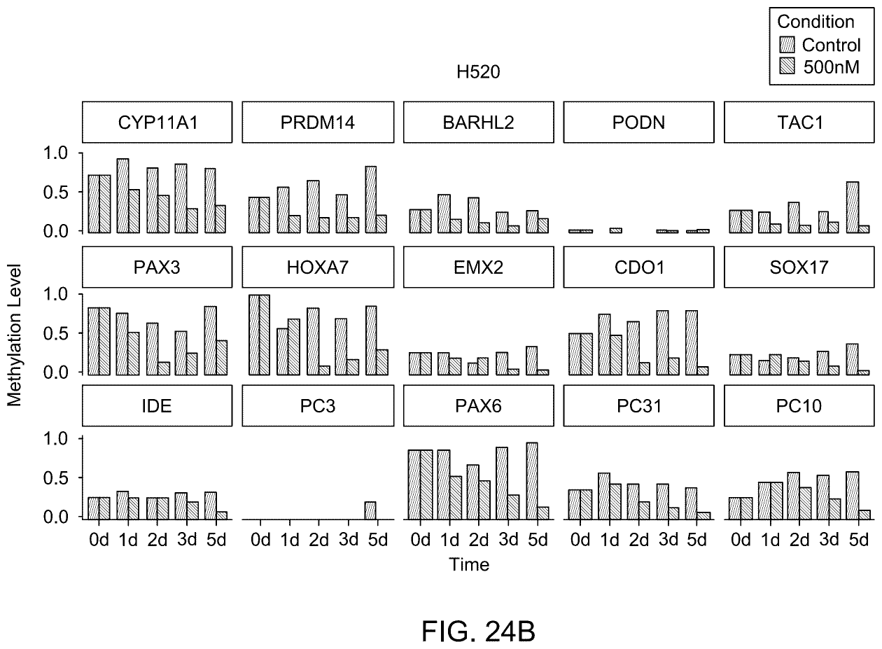

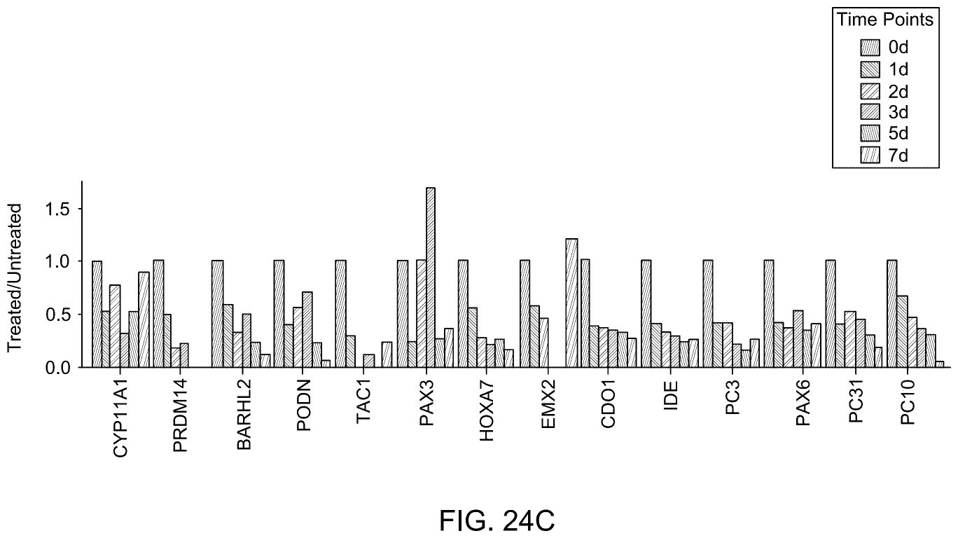

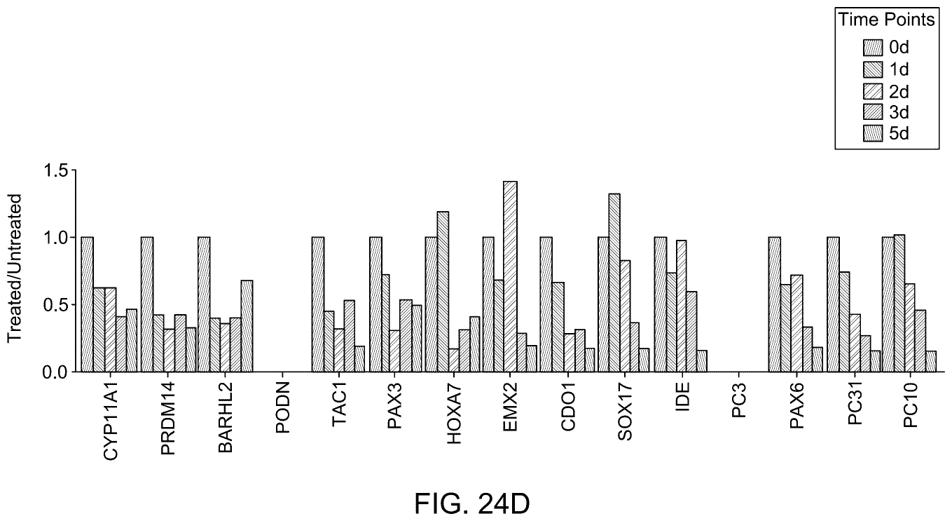

A notable application of digital PCR-based cfDNA tests is longitudinal disease progression monitoring over and after a course of treatment due to the quantification capability of the technology. Next, we sought to evaluate the performance of our assay in detecting multiple methylation biomarkers in real biological samples with a focus on the ability of our assay to quantify methylation level changes. We started with two cancer cell lines, H23 and H520, derived from lung tissues of NSCLC patients. To replicate a typical treatment monitoring scenario, we conducted hypomethylating drug treatment on the cell lines using 5-Azacytidine (5-Aza), a drug that can irreversibly bind to DNA methyltransferase after being incorporated into DNA and inhibits its activity. H520 and H23 were treated with 5-Aza at 500 nM over a course of 5 and 7 days, respectively. During the treatment course, we observed treated cells exhibited a much lower confluency rate compared with untreated cells. We confirmed that the untreated cells maintained a consistent methylation level throughout the treatment course (FIG. 24). The majority of the targets exhibited a decreasing trend in the methylation level of treated cells (FIG. 24). The variations in methylation levels among targets were likely due to the biological variations associated with the cell line. As previously reported by others, different CpG loci respond differently to hypomethylating agents depending on factors such as chromosomal location and the associated regulatory functions. H520 displayed a similar trend in that untreated cells exhibited a fluctuated but consistent methylation level throughout the course of 5-Aza treatment while the methylation levels of targets in treated cells decreased overtime (FIG. 24).

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of detecting multiple target nucleic acids in a nucleic acid sample, the method comprising:
performing a melting curve analysis using multiple probe sets under conditions sufficient to generate a melting temperature (Tₘ) detection barcode data set from partitioned sample aliquots created from the nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise:
at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or,
at least two mediator probe nucleic acids and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and,
identifying at least two different Tₘ detection barcodes in the Tₘ detection barcode data set, thereby detecting the multiple target nucleic acids.

2. The method of claim 1, comprising generating the nucleic acid sample from a protein sample by:
contacting the protein sample with a proximal binding probe set under conditions sufficient to produce a set of pairwise probe bound target proteins, wherein the proximal binding probe set comprises a plurality of proximal binding probe pairs, wherein a given proximal binding probe pair comprises a first binding probe comprising a first target protein binding moiety coupled to a first oligonucleotide that comprises a first hybridization site and a second binding probe comprising a second target protein binding moiety coupled to a second oligonucleotide that comprises a second hybridization site, wherein the first and second target protein binding moieties bind to different epitopes on a given target protein and wherein the first and second hybridization sites hybridize with one another when the first and second target protein binding moieties bind to the different epitopes on the given target protein to produce a given pairwise probe bound target protein;
extending the first and second oligonucleotides in the set of pairwise probe bound target proteins to produce extended oligonucleotides in the set of pairwise probe bound target proteins; and,
separating the extended oligonucleotides from the first and second target protein binding moieties in the set of pairwise probe bound target proteins, thereby generating the nucleic acid sample from the protein sample.

3. The method of claim 2, wherein identifying the at least two different Tₘ detection barcodes in the Tₘ data set thereby further detects multiple target proteins in the protein sample.

4. The method of claim 2, wherein the proximal binding probe set comprises an oligonucleotide-coupled antibody probe.

5. The method of claim 1, wherein the multiple probe sets comprise at least one probe set having a single probe nucleic acid that binds to a particular target nucleic acid in the partitioned sample aliquots and that comprises a melting temperature when dissociated from the particular target nucleic acid to produce a particular Tₘ detection barcode for the particular target nucleic acid.

6. The method of claim 1, wherein the multiple probe sets comprise at least one probe set having a single mediator probe nucleic acid and the reporter probe nucleic acid, wherein the single mediator probe nucleic acid comprises a first subsequence that binds to a particular target nucleic acid in the partitioned sample aliquots and a second subsequence that binds to the reporter probe nucleic acid when cleaved from the single mediator probe nucleic acid, and wherein a cleaved second subsequence from the single mediator probe nucleic acid comprises a melting temperature when dissociated from the reporter probe nucleic acid to produce a particular Tₘ detection barcode for the particular target nucleic acid.

7. The method of claim 1, wherein the nucleic acid sample comprises one or more bisulfite converted target nucleic acids and wherein the method further comprises identifying one or more methylation patterns in the bisulfite converted target nucleic acids.

8. The method of claim 1, wherein the method comprises using one or more nucleic acids comprising a nucleotide sequence selected from SEQ ID NOS: 1-30 to generate at least a portion of the Tₘ detection barcode data set.

9. The method of claim 1, comprising extending the mediator probes or a cleaved flap prior to dissociation from the universal reporter probe.

10. The method of claim 1, wherein the reporter probe nucleic acid comprises a universal reporter probe nucleic acid.

11. The method of claim 1, comprising obtaining the nucleic acid sample from a subject.

12. The method of claim 11, wherein one or more of the multiple target nucleic acids that were detected identify the subject.

13. The method of claim 11, wherein one or more of the multiple target nucleic acids that were detected are associated with at least one disease state in the subject.

14. The method of claim 13, comprising administering at least one therapy to the subject to treat the disease state in the subject.

15. A system, comprising:
a chamber configured to contain partitioned sample aliquots created from a nucleic acid sample;
a thermal modulator configured to modulate temperature in the chamber to perform a melting curve analysis using multiple probe sets under conditions sufficient to generate a melting temperature (Tₘ) detection barcode data set from the partitioned sample aliquots created from a nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise:
at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or,
at least two mediator probe nucleic acids and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and,
a detector configured to detect the Tₘ detection barcode data set; and
a controller operably connected to the temperature modulator and to the detector, which controller comprises, or is capable of accessing, computer readable media comprising non-transitory computer-executable instructions which, when executed by at least one electronic processor perform at least:
identifying at least two different Tₘ detection barcodes in the Tₘ detection barcode data set.

16. The system of claim 15, further comprising a fluid handler operably connected to the controller, wherein the fluid handler and thermal modulator are configured to generate the nucleic acid sample from a protein sample by:
contacting the protein sample with a proximal binding probe set under conditions sufficient to produce a set of pairwise probe bound target proteins, wherein the proximal binding probe set comprises a plurality of proximal binding probe pairs, wherein a given proximal binding probe pair comprises a first binding probe comprising a first target protein binding moiety coupled to a first oligonucleotide that comprises a first hybridization site and a second binding probe comprising a second target protein binding moiety coupled to a second oligonucleotide that comprises a second hybridization site, wherein the first and second target protein binding moieties bind to different epitopes on a given target protein and wherein the first and second hybridization sites hybridize with one another when the first and second target protein binding moieties bind to the different epitopes on the given target protein to produce a given pairwise probe bound target protein;
extending the first and second oligonucleotides in the set of pairwise probe bound target proteins to produce extended oligonucleotides in the set of pairwise probe bound target proteins; and,
separating the extended oligonucleotides from the first and second target protein binding moieties in the set of pairwise probe bound target proteins to thereby generate the nucleic acid sample from the protein sample.

17. The system of claim 16, wherein the non-transitory computer-executable instructions which, when executed by the electronic processor, further perform at least:
detecting multiple target proteins in the protein sample when identifying the at least two different Tₘ detection barcodes in the Tₘ detection barcode data set.

18. The system of claim 15, wherein the non-transitory computer-executable instructions which, when executed by the electronic processor, further perform at least:
performing a digital PCR technique using the nucleic acid sample prior to and/or when identifying the at least two different Tₘ detection barcodes using the thermal modulator.

19. The system of claim 15, wherein the nucleic acid sample comprises one or more bisulfite converted target nucleic acids and wherein the non-transitory computer-executable instructions which, when executed by the electronic processor, further perform at least:
identifying one or more methylation patterns in the bisulfite converted target nucleic acids using the thermal modulator and the detector.

20. A computer readable media comprising non-transitory computer-executable instructions which, when executed by at least one electronic processor, perform at least:
performing a melting curve analysis using a thermal modulator and multiple probe sets under conditions sufficient to generate a melting temperature (Tₘ) detection barcode data set from partitioned sample aliquots created from the nucleic acid sample, wherein a plurality of the partitioned sample aliquots each comprise at most one, if any, target nucleic acid and at least one of the probe sets, wherein a specified Tₘ detection barcode in the Tₘ detection barcode data set comprises one or more melting temperatures of one or more probe nucleic acids in a specified probe set when the one or more probe nucleic acids dissociate from a specified target nucleic acid, and wherein the multiple probe sets comprise:
at least two probe nucleic acids that each bind to a given target nucleic acid in the partitioned sample aliquots and that each comprise different melting temperatures when dissociated from the given target nucleic acid to produce a given Tₘ detection barcode for the given target nucleic acid, or,
at least two mediator probe nucleic acids and at least one reporter probe nucleic acid, wherein the mediator probe nucleic acids each comprise a first subsequence that binds to a given target nucleic acid in the partitioned sample aliquots and a second subsequence that binds to the reporter probe nucleic acid when cleaved from the mediator probe nucleic acids, and wherein cleaved second subsequences from the at least two mediator probe nucleic acids each comprise different melting temperatures when dissociated from the reporter probe nucleic acid to produce a given Tₘ detection barcode for the given target nucleic acid; and,
identifying at least two different Tₘ detection barcodes in the Tₘ detection barcode data set using a detector.
